(19) United States
(12) Patent Application Publication
(10) Pub. No.: US 2025/0354195 A1
(43) Pub. Date: Nov. 20, 2025

(54) METHOD OF SCREENING

(71) Applicant: UNIVERSITY OF DUNDEE, Dundee (GB)

(72) Inventors: William FARNABY, Dundee (GB); Andreas HOLMQVIST, Dundee (GB); Nur KOCATURK, Dundee (GB)

(21) Appl. No.: 19/209,669

(22) Filed: May 15, 2025

Related U.S. Application Data
(60) Provisional application No. 63/647,763, filed on May 15, 2024.

(51) Int. Cl. C12Q 1/48; C07D 487/10; C12N 9/10

(52) U.S. Cl. CPC C12Q 1/48; C07D 487/10; C12N 9/104

(57) ABSTRACT

The present disclosure relates to a method of screening reagents to assess their suitability in forming a bifunctional compound, the method comprising contacting a linker of formula (I) with two molecules, and optionally analysing the resultant mixture for formation of the bifunctional compound. The disclosure also concerns a linker of formula (1), the use of the linker in the manufacture of bifunctional compounds, and kits comprising the linker.

### FIELD

The present disclosure relates to a method of screening reagents to assess their suitability in forming a bifunctional compound, the method comprising contacting a linker of formula (I) with two molecules, and optionally analysing the resultant mixture for formation of the bifunctional compound. The disclosure also concerns a linker of formula (1), the use of the linker in the manufacture of bifunctional compounds, and kits comprising the linker.

### BACKGROUND

Bifunctional compounds, in accordance with the present disclosure (or bifunctional molecules) are compounds that comprise two functional moieties joined by a linker. The functional moieties are often bioactive molecules, such as ligands that interact with biological systems. Many different chemical groups can be used as a linker. Bifunctional compounds are often used in ‘induced proximity’, which is a technique that facilitates or enables the interaction of two or more biological molecules, such as proteins, by bringing them into proximity. There are several mechanisms and modalities of induced proximity, such as proteolysis-targeting chimera, phosphorylation-targeting chimera, deubiquitinase-targeting chimera, lysosome-targeting chimera, and autophagy-targeting chimera. Generally speaking, these modalities involve the recruitment of a biological system to a target protein. This is often achieved using a bifunctional compound that comprises a first ligand for the biological system which is joined to a second ligand through a linker, wherein the second ligand is a ligand for the target protein. For example, in proteolysis-targeting chimeras, the bifunctional compound comprises a ligand for an E3 ubiquitin ligase, which is recruited to the target protein—the target protein is then ubiquitinated, and subsequently degraded by the proteasome. Proximity inducing modalities are promising techniques for medicine and the wider biological sciences.

The synthesis of bifunctional compounds is typically a linear, stepwise process. An example process is: 1) a first ligand is reacted with a linker molecule, capable of linking two ligands; 2) the resultant molecule comprising both the linker and the first ligand is isolated; 3) the linker moiety is then functionalised (e.g., removal of a protecting group, or activation of a reactive group); 4) the resultant functionalised molecule is isolated; 5) a second ligand is reacted with the functionalised linker moiety, thereby forming the bifunctional compound; and 6) the bifunctional compound is isolated. Typically, each of these steps require individual experimental set-up and purification (e.g., work-up and/or chromatography) by the operator, resulting in generally time-consuming, labour-intensive, and costly processes. That is to say, they are operatively complex. Additionally, such processes are typically not amenable to parallel synthesis, that is, the simultaneous synthesis of an array of compounds.

Linear processes limit the potential for structural diversification in bifunctional compounds, as modification must be made sequentially; each new variant requires a separate step, compounding the complexity and length of the synthesis with each derivative introduced. As the biological screening of bifunctional compounds (e.g. to test their efficacy in proximity based modalities) is often performed in a high-throughput process, their chemical synthesis may be a considerable bottleneck in the development timeline. Instead, it has been proposed that the synthesis of bifunctional compounds is adapted to high-throughput methods that are more compatible with, for example, plate-based biological testing, so called ‘direct-to-biology’ (D2B) methods. D2B methods may be considered a method of screening reagents to assess their suitability in forming bifunctional by performing the chemistry in a single reaction vessel (i.e. a one-pot synthesis), such as a single-well of a multi-well plate wherein the reagents are varied between wells. The biological efficacy of the bifunctional compound itself may then be screened for the particular modality of interest, sometimes also via plate-based screening. It is important to note that one bifunctional compound may be efficacious in a particular modality and/or for a particular target protein, but not necessarily for another.

A recent example of a D2B method is the amide coupling platform described by Hendrick et al., in ACS Med. Chem. Lett., 2022, 13, 1182. Using a plate-based system, linkers were varied in a single-well, three-step process involving: 1) an amide coupling of a protected diamine linker with a first ligand (comprising an activated carboxylic acid); 2) removal of the linker protecting group; and 3) an amide coupling of the deprotected linker with a second ligand (also comprising an activated carboxylic acid). The existing D2B multi-step syntheses like that used here have a negative impact on process time and cost. Additionally, the multiple reaction steps of these known D2B methods can result in a complex mixture of reagents and compounds, sometimes resulting in low purity of bifunctional compounds. The linker used by Hendrick et al. comprises two equivalent reactive moieties (amines) to react with the ligands—this approach typically requires the use of protecting groups, and can limit overall structural diversity.

A further example of a D2B method is the Ugi multi-component reaction (MCR) platform described by Wang et al., in Nat. Commun., 2023, 14, 8437. Using a plate-based system, the authors kept the linker and a first ligand constant whilst varying the second ligand, and the nature of the linkage point. This was achieved through plating a nitrile functionalised linker—pre-attached to the first ligand—with several small components that could react with the nitrile group via an Ugi reaction; the Ugi reaction fashioned the second ligand. In this approach, the structural diversification of the bifunctional compound is essentially limited to only the second ligand, due to the complexity and relative lack of selectivity in Ugi MCRs.

Thus, there is a need in the art for alternative D2B methods, preferably methods that are operatively simple (i.e., time, labour, and cost effective) and enable high levels of structural diversity of bifunctional compounds. In particular, methods that enable multiple orthogonal, rather than single or equivalent reactive moieties to react with the ligands to maximise library diversity and/or hit rate are needed. The present disclosure seeks to address one or more of these needs.

### SUMMARY

The present investigators have developed an operatively simple direct-to-biology (D2B) method of producing bifunctional compounds with high levels of structural diversity. The method is a “one-pot” method, thus enabling, for example, an array of linkers and molecules to be run on a multi-well plate, as each well of the plate may constitute an independent reaction vessel for the method with different reagents in each.

This enables the method to potentially generate a large and diverse library of bifunctional compounds, hence the method is surprisingly effective for the screening of reagents to assess their suitability in forming bifunctional compounds. The investigators have found that this can be achieved using a linker cleverly designed to comprise two orthogonally reactive moieties. That is to say, the two moieties have reactivities independent of each other and thus each react efficiently and selectively with two molecules, one of which comprises a moiety intended for reacting with one of the two orthogonally reactive moieties, and the other of which comprises a moiety intended for reacting with the other of the two orthogonally reactive moieties.

The present investigators have identified several different pairings of orthogonally reactive moieties, and additionally have identified several different suitable complementary moieties comprised in molecules to react with the orthogonally reactive moieties. The investigators have found that the linkers are surprisingly tolerant of a variety of molecules and reaction conditions. Additionally, the method presented herein is a “one-pot” process performed in a single reaction vessel, such as a single-well of a plate. The method requires no additional functionalisation step (e.g., protecting group removal) or isolation step, e.g. chromatography, enables biological screening directly on the crude bifunctional compounds produced, and allows for simultaneous variation of the linker and the ligands (‘molecules’).

Therefore, in a first aspect there is provided a one-pot method comprising contacting: (a) a linker comprising two orthogonally reactive moieties; and (b) two molecules, one of which comprises a moiety for reacting with one of the two orthogonally reactive moieties, and the other of which comprises a moiety for reacting with the other of the two orthogonally reactive moieties.

The method may, and typically does, further comprise analysing the resultant mixture for formation of a bifunctional compound comprising each of the two molecules linked together by the linker.

Through their development of the methods presented herein, the investigators have designed and prepared several different linker compounds comprising orthogonally reactive moieties, suitable for forming bifunctional compounds in accordance with the present disclosure.

Therefore, in a second aspect, there is provided a linker of formula (1):

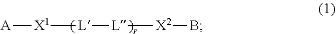

wherein: A is

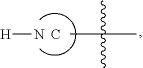

wherein ring C is a bicyclic spiro moiety comprising 4- to 6-membered aliphatic N-heterocyclic rings and optionally substituted with one or more selected from halo, C₁₋₆alkyl, C₁₋₆haloalkyl, C₁₋₆alkoxy, hydroxy, aryl, heteroaryl, and C₁₋₆haloalkoxy, and B is ethynyl or a nucleofuge; each X¹ and X² is optionally present and is any one selected from the group consisting of O(CH₂)ₛ and N(C₁₋₆alkyl)(CH₂)ₛ; each L′ is independently selected from the group consisting of O(CH₂)ₜ, CH₂, heterocyclylene, arylene, and cycloalkylene, each optionally substituted with one or more substituents selected from halo, C₁₋₆alkyl, C₁₋₆haloalkyl, C₁₋₆alkoxy, hydroxy, aryl, heteroaryl, and C₁₋₆haloalkoxy; L″ is optionally present and is selected from —O— and —N(C₁₋₆alkyl)-; and r is an integer from 1 to 20, s is an integer from 0 to 4, and t is an integer from 1 to 4.

The present investigators have found that the linkers of the second aspect are particularly effective in the manufacture of bifunctional compounds, as well as in a variety of uses, including use in the manufacture (e.g. one-pot manufacture) of bifunctional compounds and/or use in targeted protein degradation or stabilisation.

Therefore, in a third aspect, there is provided a method of manufacturing a bifunctional compound, optionally as a one-pot method, the method comprising:
(i) contacting:
(a) a linker of the second aspect; and
(b) two molecules, one of which comprises a moiety for reacting with one of A and B, and the other of which comprises a moiety for reacting with the other of A and B;
and optionally:
(ii) analysing the resultant mixture for formation of a bifunctional compound comprising each of the two molecules linked together by the linker.

In a fourth aspect, there is provided a method of targeted protein degradation or stabilisation, the method comprising:
(i) contacting:
(a) a linker of the second aspect; and
(b) two molecules, one of which comprises a moiety for reacting with one of A and B, and the other of which comprises a moiety for reacting with the other of A and B, and wherein one of the molecules comprises a target protein binder and the other comprises an E3 ubiquitin ligase or a deubiquitinase binder;
(ii) analysing the resultant mixture for formation of a bifunctional compound comprising each of the two molecules linked together by the linker; and
(iii) contacting the bifunctional compound with the target protein.

The present investigators have also recognised that the linkers of the second aspect can be provided in a kit with one or two molecules for reacting with the orthogonally reactive moieties of the linker, thus may provide to a user components for manufacturing bifunctional molecules, and/or for screening reagents to assess their suitability in forming bifunctional compounds.

Therefore, in a fifth aspect, there is provided a kit comprising: (i) a linker according to the second aspect; and (ii) a molecule comprising a moiety for reacting with A and/or a molecule comprising a moiety for reacting with B.

Additionally, the present investigators have designed and prepared several molecules comprising a binder for cereblon (CRBN), and a moiety for reacting with one of the two orthogonally reactive moieties of the linkers of the second aspect.

Therefore, in a sixth aspect, there is provided molecules of formula (VIIa):

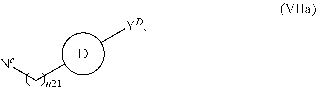

wherein:
   N^{c} is a nucleofuge;
   n21 is an integer selected from 1 to 10;
   D is an aromatic or heteroaromatic ring, optionally substituted with halo, C₁₋₆alkyl, C₁₋₆haloalkyl, C₁₋₆alkoxy, and C₁₋₆haloalkoxy; and
Y^{D} is selected from

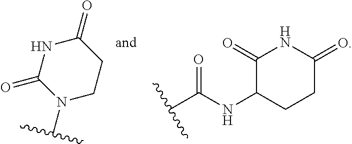 [SMILES: *N1CCC(=O)NC1=O ; *C(=O)NC1CCC(=O)NC1=O]

### BRIEF DESCRIPTION OF THE FIGURES

FIG. 1: Results of a plate-based array of linkers and molecules screened for their suitability in forming a bifunctional compound. Each well is a separate one-pot method run on a multi-well plate. Lighter shades indicate higher purity of the resultant bifunctional compounds; darker shades indicate lower purity.

FIG. 2: Results of an alternative plate-based array of linkers and molecules screened for their suitability in forming a bifunctional compound.

FIG. 3: Results of comparative biological testing of ‘crude’ bifunctional compounds produced using the methods disclosed herein versus their conventionally purified counterparts. “Compound 1” and “Compound 2” refer to the bifunctional compounds of well ‘D7’ and well ‘D1’ of FIG. 1, respectively.

### DETAILED DESCRIPTION

### Definitions

In the discussion that follows, reference is made to a number of terms, which have the meanings provided below, unless a context indicates to the contrary. The nomenclature used herein for defining compounds, in particular the compounds according to the disclosure, is in general based on the rules of the IUPAC organisation for chemical compounds, specifically the “IUPAC Compendium of Chemical Terminology (Gold Book)”. For the avoidance of doubt, if a rule of the IUPAC organisation is in conflict with a definition provided herein, the definition herein is to prevail. Furthermore, if a compound structure is in conflict with the name provided for the structure, the structure is to prevail.

The term “comprising” or variants thereof is to be understood herein to imply the inclusion of a stated element, integer or step, or group of elements, integers or steps, but not the exclusion of any other element, integer or step, or group of elements, integers or steps.

The term “consisting” or variants thereof is to be understood to imply the inclusion of a stated element, integer or step, or group of elements, integers or steps, and the exclusion of any other element, integer or step or group of elements, integers or steps.

The term “contacting” is used herein to refer to any one or more of the acts of combining, such as reacting, mixing, stirring, slurrying, blending, dissolving, incubating, passing over, flowing over, or otherwise, in any order, and for any length of time.

The term “one-pot” method is used herein to refer to a method wherein the steps of the method, e.g. the chemical synthesis steps (reactions), are carried out in a single reaction vessel, such as a single-well of a multi-well plate, and the reagents for each step are added, optionally sequentially, to the same vessel without isolation and/or purification between steps. Some one-pot methods may be described as “telescopic”.

Generally, telescopic one-pot methods refer to one-pot methods comprising sequential chemical reactions whereby the product of one reaction is the necessary starting material of the next reaction, expressly excluding isolation or purification steps in-between reaction steps, and optionally excluding (i) any change in reaction conditions; and/or (ii) the addition of further reagents or starting materials. That is to say, telescopic one-pot methods generally require no or minimal intervention from the operator between reaction steps.

The term “bifunctional compound”, as used herein, refers to compounds that comprise two functional moieties joined by a linker. The functional moieties may confer or impart any one or more functions on the compound. Often, the functional moieties comprise ligands (“binders”) that interact with biological systems. Further broad classes of functional moieties include but are not limited to: tags, such as protein tags; detectable labels (such as fluorescent groups); immobilising groups; solubilising groups; and reactive handles. The linker of a bifunctional compound may be any chemical group capable of joining the functional moieties. In some cases, the linker is specifically chosen or designed to confer particular properties on the bifunctional compound and/or the method to manufacture the bifunctional compound.

The term “ethynyl” refers to a univalent group derived from ethyne by removal of one hydrogen atom, wherein ethyne is the alkyne HC═CH.

The term “nucleofuge” (sometimes “leaving group”) herein refers to an atom or group of atoms (charged or uncharged) that may become detached from the residual or main part of a compound as part of a reaction. The term nucleofuge may specifically refer to leaving groups that depart with a pair of electrons in a heterolytic bond cleavage. Atoms or groups that may act as leaving groups in reactions include but are not limited to halo, sulfonium, sulfonyl, sulfonate, sulfinyl, sulfinate, dinitrogen, dialkyl ether, water, nitrate, phosphate, thioether, amine, and ammonia. Further examples of nucleofuges include 4-methylphenyl-1-sulfonate (toluenesulfonate, or “tosylate”), 4-bromophenyl-1-sulfonate (or “brosylate”), 4-nitrophenyl-1-sulfonate (or “nosylate”), methanesulfonate (or “mesylate”), trifluoromethanesulfonate (or “triflate”), 2,2,2-trifluoroethyl-1-sulfonate (or “tresylate”), 5-(dimethylamino)naphthalene-1-sulfonate (or “dansylate”), iodo, bromo, and chloro.

The term “nucleophile” herein refers to an atom or group of atoms that donates a pair of electrons to form chemical bonds, often through a nucleophilic substitution reaction. Nucleophilic substitution is a chemical reaction wherein a nucleophile (an electron pair donor) donates a pair of electrons to an electropositive moiety, which is or was previously bonded to a nucleofuge (an electron pair acceptor). The bond between the electropositive moiety and the nucleofuge breaks, with the electron pair from the bond being transferred to the nucleofuge. A bimolecular nucleophilic substitution reaction is often referred to as an “S_{N}2” reaction, wherein bond forming (to the nucleophile) and bond breaking (to the nucleofuge) occurs in a concerted fashion. A unimolecular nucleophilic substitution reaction is often referred to as an “S_{N}1” reaction, wherein the abovementioned bond forming and bond breaking steps are sequential. A nucleophilic substitution reaction that occurs at a (hetero)aromatic carbon atom (sp²-hybridised), often referred to as an “S_{N}Ar” reaction, can occur via a variety of reaction mechanisms. Often, S_{N}Ar reactions occur via an addition-elimination reaction mechanism, typically at electropositive carbon atoms. The nucleofuge is often halo, typically bromo, chloro or fluoro, more typically chloro or fluoro. Examples of nucleophiles for a variety of nucleophilic substitution reactions include but are not limited to amines, thiols/thiolates, organometallic reagents (such as Grignard reagents, organolithium reagents, and Gilman reagents), enols/enolates, alcohols, and alkoxides.

The term “aromatic” refers to a cyclically conjugated molecular entity with a stability (due to delocalisation) significantly greater than that of a hypothetical localised structure. The Hückel rule is often used in the art to assess aromatic character; monocyclic planar (or almost planar) systems of trigonally (or sometimes diagonally) hybridised atoms that contain (4n+2) π-electrons (where n is a non-negative integer) will exhibit aromatic character. The rule is generally limited to n=0 to 5.

The term “heteroaryl” refers to univalent groups derived from heteroaromatic compounds (that is, aromatic compounds comprising one or more atoms selected from nitrogen, oxygen, and sulfur) by the removal of a hydrogen atom from any one carbon atom or heteroatom. Common examples of heteroaryl groups include but are not limited to pyrrole, imidazole, pyrazole, triazole, tetrazole, furan, thiophene, oxazole, isothiazole, thiazole, thiadiazole, pyridine, pyridazine, pyrimidine, pyrazine, triazine, indole, benzimidazole, azaindole, benzofuran, and benzothiophene.

The term “aliphatic” refers to hydrocarbon compounds that are not aromatic. Aliphatic compounds may be linear or cyclic, and they may be branched. They may comprise unsaturated bonds, i.e., carbon-carbon double bonds.

The term “N-heterocycle” refers to mono or polycyclic aliphatic or aromatic compounds that comprise one or more nitrogen atoms. Examples of N-heterocycles include but are not limited to piperidine, piperazine, diazepane (such as 1,4-diazepane), diazaspiro[3.3]heptane (such as 2,6-diazaspiro[3.3]heptane), diazaspiro[3.4]octane (such as 2,6-diazaspiro[3.4]octane), azaspiro[3.3]heptane (such as 2-azaspiro[3.3]heptane), azaspiro[3.4]octane (such as 2-azaspiro[3.4]octane), aziridine, azetidine, diazetidine, azetidinone, pyrrolidine, pyrroline, pyrrole, pyrazolidine, imidazolidine, pyrazoline, imidazoline, pyrazole, imidazole, triazole, tetrazole, oxazole, isoxazole, isothiazole, succinimide, oxazolidone, pyridine, pyridazine, pyrimidine, pyrazine, triazine, morpholine, thiomorpholine, indoline, indole, isoindole, indolizine, indazole, benzimidazole, azaindole, azaindazole, purine, benzisoxazole, adenine, guanine, quinoline, isoquinoline, naphthyridine, pteridine, carbazole, azaadamantane, and azepine.

The term “halo” refers to a halogen radical. Typically, halo refers to any selected from fluoro, bromo, chloro and iodo. In some cases, halo refers to fluoro.

The term “alkyl” is well known in the art and defines univalent groups derived from alkanes by removal of a hydrogen atom from any carbon atom, wherein the term “alkane” is intended to define acyclic branched or unbranched hydrocarbons having the general formula CₙH₂ₙ₊₂, wherein n is an integer ≥1. Alkyl groups may be C₁₋₆alkyl groups, including but not limited to methyl, ethyl, n-propyl, iso-propyl, n-butyl, sec-butyl, iso-butyl and tert-butyl, n-pentyl, 1-methyl-butyl, 2-methyl-butyl, 3-methyl-butyl, 1,2-dimethyl-propyl, 1,1-dimethyl-propyl, neo-pentyl, and n-hexyl. In some cases, alkyl groups are C₁₋₄ alkyl groups. C₁₋₄alkyl refers to any selected from the group consisting of methyl, ethyl, n-propyl, iso-propyl, n-butyl, sec-butyl, iso-butyl and tert-butyl.

The term “haloalkyl” is also well known and defines univalent groups derived from alkyl groups by replacement of one or more hydrogen atoms from one or more carbon atoms with a halo group. Haloalkyl groups may comprise one or more different types of halo. For example, one or more independently selected from fluoro, chloro, bromo and iodo. In some cases, the haloalkyl is a fluoroalkyl. Haloalkyl groups may be C₁₋₆haloalkyl groups. In some cases, haloalkyl groups are C₁₋₆haloalkyl groups, C₁₋₄alkyl groups, C₁₋₃haloalkyl groups, or C₁₋₂haloalkyl groups. Non-limiting examples of haloalkyl groups are trifluoromethyl, trifluoroethyl, perfluoroethyl, chloroethyl, bromoethyl, iodoethyl, chlorofluoroethyl, bromofluoroethyl, and iodofluoroethyl.

The term “alkoxy” defines univalent groups derived from alcohols by removal of a hydrogen atom from an —OH group, wherein the term “alcohol” is intended to define groups derived from alkanes by the replacement of a hydrogen atom with a hydroxy group. C₁₋₆alkoxy refers but is not limited to methoxy, ethoxy, n-propoxy, iso-propoxy, n-butoxy, sec-butoxy, iso-butoxy, tert-butoxy, pent-1-oxy, pent-2-oxy, pent-3-oxy, neo-pentoxy, hex-1-oxy, hex-2-oxy, and hex-3-oxy. C₁₋₄alkoxy refers to any one selected from the group consisting of methoxy, ethoxy, n-propoxy, iso-propoxy, n-butoxy, sec-butoxy, iso-butoxy and tert-butoxy.

The term “haloalkoxy” defines univalent groups derived from alkoxy groups by removal of one or more hydrogen atoms with a halo group. Haloalkoxy groups may comprise one or more different type of halo. For example, one or more independently selected from fluoro, chloro, bromo and iodo. In some cases, the haloalkoxy is a fluoroalkoxy. Haloalkoxy groups may be C₁₋₆haloalkoxy groups. Non-limiting examples of haloalkoxy groups are trifluoromethoxy, difluoromethoxy, fluoromethoxy, difluoroethoxy, trifluoroethoxy, perfluoroethoxy, chloroethoxy, bromoethoxy, iodoethoxy, chlorofluoroethoxy, bromofluoroethoxy, and iodofluoroethoxy.

The term “heterocyclylene” defines a divalent group derived from a heterocycle by the removal of two hydrogen atoms from one or two atoms of the heterocycle. Examples of heterocyclylene groups include but are not limited to pyrrolylene, imidazolylene, pyrazolylene, triazolylene, tetrazolylene, furanylene, thiophenylene, oxazolylene, isothiazolylene, thiazolylene, thiadiazolylene, pyridinylene, pyridazinylene, pyrimidinylene, pyrazinylene, triazinylene, indolylene, benzimidazolylene, azaindolylene, benzofuranylene, benzothiophenylene, pyrolidinylene, pyrrolinylene, tetrahydrofuranylene, tetrahydrothiophenylene, piperidinylene, piperazinylene, tetrahydropyranylene, thianylene, dithianylene, morpholinylene, and thiomorpholinylene.

The term “arylene” is understood to refer to divalent groups derived from arenes (aromatic compounds such as benzene, naphthalene, fluorene, anthracene, and phenanthrene) by the removal of two hydrogen atoms from any one or two carbon atoms.

In some cases, arylene refers to phenylene (derived from benzene), naphthylene, fluorenylene, anthracenylene, and phenanthrenylene. The term “aryl” is therefore understood to refer to univalent groups derived from arenes by the removal of one hydrogen atom from any carbon atom, such as phenyl, naphthyl, fluorenyl, anthracenyl, and phenanthrenyl.

The term “cycloalkylene” refers to divalent groups derived from a cyclic alkane by removal of two hydrogen atoms, and, for example, includes cyclohexylene, cycloheptylene, cyclooctylene, cyclononylene, and cyclodecylene. Cycloalkylene groups may comprise one or more rings, and include fused or spiro-cyclic groups.

The term “bicyclic spiro” refers to a ring system wherein two rings share a single common carbon atom, sometimes referred to as the “spiro atom”. The rings may independently be cyclic alkanes or heterocycles, and each ring may comprise the same or a different number of atoms.

The term “sulfonate” used herein refers to univalent groups derived from sulfonic acids by removal of the acidic hydrogen atom. Sulfonate groups are well-known in the art as effective nucleofuges (leaving groups). Examples of common sulfonates include but are not limited to 4-methylphenyl-1-sulfonate (toluenesulfonate, or “tosylate”), 4-bromophenyl-1-sulfonate (or “brosylate”), 4-nitrophenyl-1-sulfonate (or “nosylate”), methanesulfonate (or “mesylate”), trifluoromethanesulfonate (or “triflate”), 2,2,2-trifluoroethyl-1-sulfonate (or “tresylate”), and 5-(dimethylamino)naphthalene-1-sulfonate (or “dansylate”).

The term “sulfonium” used herein refers to the cationic group R₂S⁺—, wherein each R is independently H or a substituent, such as alky or aryl. Sulfonium groups are good nucleofuges. Some examples of sulfonium groups include but are not limited to dimethylsulfonium and diphenylsulfonium.

The term “carboxylic acid” used herein refers to the univalent group derived from formic acid by the removal of the hydrogen atom from the carbon atom, often depicted as —COOH or —CO₂H. The term “acyl chloride” used herein refers to the univalent group derived from formyl chloride by the removal of the hydrogen atom, often depicted as —COCl. The term “sulfonyl chloride” used herein refers to the univalent group derived from sulfuryl chloride (formula: SO₂Cl₂) by the removal of one chlorine atom, often depicted as —SO₂Cl. The term “activated carboxylic acid” refers to carboxylic acid groups that have been modified to increase their reactivity, typically in amide coupling reactions. Examples of activated carboxylic acids include but are not limited to acid anhydrides, esters, carbodiimides, nitrophenyl esters, and N-hydroxysuccinimide (NHS) esters.

The term “pyridinene” refers to the divalent group derived from pyridine by the removal of two hydrogen atoms.

The term “thiol” is well known in the art and defines the univalent group derived from hydrogen sulfide by the removal of one hydrogen atom. Thiol groups are often depicted as —SH. Thiol groups are well known in the art as effective nucleophiles.

The term “hydroxy” is well known in the art and defines the univalent group derived from water by the removal of one hydrogen atom. Hydroxy groups are often depicted as —OH. Hydroxy groups are well known in the art as effective nucleophiles.

The term “amino” refers herein to an —NR¹R² group, wherein R¹ and R² are independently H or hydrocarbon-derived substituents, such as alkyl, alkenyl, alkynyl, carbocycle, and their substituted counterparts. R¹ and R² may be unsubstituted. In some cases, R¹ and R² are joined together. For example, R¹ and R² may be joined together in such a way that the nitrogen atom of —NR¹R² is part of a ring system. When R¹ and R² are H, the compound comprising the amino group may be referred to as a primary amine. When one of R¹ or R² is H, the compound may be referred to as a secondary amine. When neither R¹ nor R² are H, the compound may be referred to as a tertiary amine. Amino groups are well known in the art as effective nucleophiles. Typically, “amino” refers to primary or secondary amino groups.

The term “biological molecule” or “biomolecule” is well known in the art and relates to any molecule present in organisms that is involved in one or more (typically biological) processes. Biological molecules may include but are not limited to proteins, peptides, antibodies, antigens, carbohydrates, lipids, nucleic acids, polynucleotides, vitamins, amino acids, and hormones. Biological molecules may be extracted from their natural source. They may be produced by synthetic or biotechnological means. Biological molecules may be of an unnatural origin, have no known biological purpose, or may not be known to be involved in any biological process. They may be engineered or produced in such a way to differ from their natural counterparts. In some cases, biological molecules are proteins.

The term “ligand”, used interchangeably with “binder”, is used herein to refer to molecules (sometimes “compounds”) that can bind to biomolecules to form a complex—a complex is understood to be a stable association between two or more molecules to form a single unit. The ligand may bind reversibly through non-covalent interactions, or irreversibly through the formation of a covalent bond. The ligand molecule may be a small-molecule, or it may be a macromolecule, such as a protein. The ligand may be a natural ligand for a biological molecule, or it may be unnatural. The ligand may also be a part of a molecule. That is to say, a ligand molecule may comprise parts that bind and other parts that do not, or parts that bind to particular biological molecules and other parts that bind to other biological molecules. The ligand may only bind to one or a few biological molecules, or it may be promiscuous. As a result of the ligand binding to a biological molecule, the biological molecule may undergo some conformational change, recruit another biological molecule, be prevented from binding another ligand or substrate, or otherwise be affected in one or more ways.

The term “kit” is used herein to refer to a product containing the different components necessary for making the compounds or carrying out the uses and methods of the present disclosure. The different components may be provided within packaging so as to allow their transport and storage. The kit may comprise the different components in separate vessels or containers.

### Methods

As described above, in a first aspect there is provided a one-pot method comprising contacting:
(a) a linker comprising two orthogonally reactive moieties; and
(b) two molecules, one of which comprises a moiety for reacting with one of the two orthogonally reactive moieties, and the other of which comprises a moiety for reacting with the other of the two orthogonally reactive moieties.

For the avoidance of doubt, where a moiety is “for reacting with one of the two orthogonally reactive moieties”, the moiety is one that would itself, when not part of the molecule concerned, react with one of the two orthogonally reactive moieties. Thus, the moiety is suspected to or is considered by one of skill to potentially (i.e. it is possible that it might) react with one of the two orthogonally reactive moieties.

The method may, and typically does, further comprise analysing the resultant mixture for formation of a bifunctional compound comprising each of the two molecules linked together by the linker.

As described above, the methods of the first aspect of the invention are suitable for screening reagents to assess their suitability in forming a bifunctional compound. As the method is a one-pot method (defined above), the method can be performed in, for example, a single-well of a multi-well plate. Each well of the plate is then able to be sampled for analysing, wherein the analysing is to detect the amount and/or purity of the bifunctional compound that may be formed in the well. In some embodiments, the analysing comprises analytical techniques, such as liquid chromatography-mass spectrometry (LCMS), UV-vis spectroscopy, nuclear magnetic resonance (NMR) spectroscopy, gas chromatography-mass spectrometry (GCMS), high performance liquid chromatography (HPLC), and thin layer chromatography (TLC). Some analytical techniques, for example LCMS and HPLC, may use UV-vis detectors to quantify analytes. Typically, where the method further comprises analysing the resultant mixture, the analysing comprises LCMS with a UV-vis detector.

As described above, the linker comprises two orthogonally reactive moieties. The term “linker”, unless specified otherwise, is used herein to describe any chemical moiety capable of linking two orthogonally reactive moieties, and hence also capable of linking the two molecules that react with the reactive moieties. The skilled person is familiar with many different chemical groups and functionalities that are capable of linking molecules. Specific examples are described below.

As described above, the reactive moieties are “orthogonally reactive”. That is to say, the two moieties have reactivities independent of each other and thus may each react efficiently and selectively with two molecules, one of which comprises a moiety for reacting with one of the two orthogonally reactive moieties, and the other of which comprises a moiety for reacting with the other of the two orthogonally reactive moieties.

In some embodiments, the linker comprises a basic amine group. For example, the linker may comprise

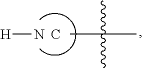

which may be a basic amine group. A basic amine group is a moiety that comprises an amino group, wherein the amino group is capable of donating a lone pair of electrons (otherwise accepting a proton, H⁺) to act as a base. The skilled person will recognise amino groups that are basic amine groups. Examples of basic amine groups are alkylamines (such as triethylamine and N,N-diisopropylethylamine), N-heterocycles (such as azetidine, diazetidine, pyrrolidine, piperidine, piperazine), azaspiroalkanyls (such as 2H-2-azaspiro[3.3]heptanyl, 2H-2-azaspiro[3.4]octanyl, and 2H-2-azaspiro[3.4]octanyl), and diazaspiroalkanyls (such as 2H-2,6-diazaspiro[3.3]heptanyl, 2H-2,6-diazaspiro[3.4]octanyl, and 2H-2,7-diazaspiro[3.4]octanyl).

In some embodiments, the method further comprises a purification step, wherein the purification comprises an ion-exchange system, typically a cation-exchange system such as SCX (strong-cation exchange). Cation exchange systems typically comprise a bead matrix solid support, often comprising cross-linked polystyrene or silica gel, wherein the solid support comprises acidic groups (such as sulfonic acids, phosphonic acids, and carboxylic acids). The acidic groups are able to bind to basic groups (often basic amine groups) on a compound to be purified that has been loaded on to the solid support; impurities that do not bind may then be washed away, for example, with an organic solvent; the purified compound may then be eluted from the solid support by changing the pH of the system. Thus, in such embodiments (where the method further comprises a purification step comprising an ion-exchange system), the linker comprises

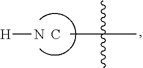

which is a basic amine group.

The investigators have found that, where the resultant mixture from the method of the first aspect comprises a proteolysis targeting chimera, it can be contacted with a target protein and/or a cell, for example to assess its bioactivity, without further purification. The investigators have found that the results of bioactivity analysis are surprisingly similar when the resultant mixture is analysed either with or without purification. Accordingly, in some embodiments, the resultant mixture is not purified, e.g. prior to analysing its bioactivity.

In some embodiments, the linker is of formula (I):

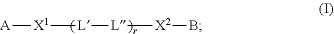

and
one of the two molecules comprises a moiety suitable for reacting with A, and the other comprises a moiety suitable for reacting with B;
wherein:
A is

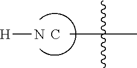

and B is ethynyl;
A is a nucleofuge and B is ethynyl; or
A is

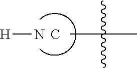

and B is a nucleofuge;
and wherein:
ring C is an aliphatic N-heterocycle optionally substituted with one or more selected from halo, C₁₋₆alkyl, C₁₋₆haloalkyl, C₁₋₆alkoxy, hydroxy, aryl, heteroaryl, and C₁₋₆haloalkoxy;
each X¹ and X² is optionally present and is any one selected from the group consisting of O(CH₂)ₛ and N(C₁₋₆alkyl)(CH₂)ₛ;
each L′ is independently selected from the group consisting of O(CH₂)ₜ, CH₂, heterocyclylene, arylene, and cycloalkylene, each optionally substituted with one or more substituents selected from halo, C₁₋₆alkyl, C₁₋₆haloalkyl, C₁₋₆alkoxy, hydroxy, aryl, heteroaryl, and C₁₋₆haloalkoxy;
L″ is optionally present and is selected from —O— and —N(C₁₋₆alkyl)-;
r is an integer from 1 to 20, s is an integer from 0 to 4, and t is an integer from 1 to 4.

In some embodiments, the linker is of formula (IIa) or (IIb):

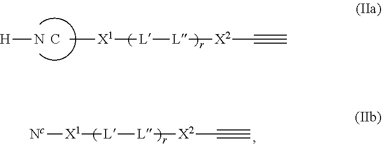

wherein:
N^{c} is the nucleofuge.

As described above, in some embodiments, A is

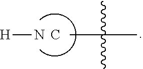

That is to say, A comprises an aliphatic N-heterocycle (ring C), wherein A comprises at least one secondary amino group, depicted by the ‘H—N’ of the structure above. Examples of suitable monocyclic aliphatic N-heterocycles include but are not limited to piperidine, piperazine, diazepane (such as 1,4-diazepane), diazaspiro[3.3]heptane (such as 2,6-diazaspiro[3.3]heptane), diazaspiro[3.4]octane (such as 2,6-diazaspiro[3.4]octane), azaspiro[3.3]heptane (such as 2-azaspiro[3.3]heptane), azaspiro[3.4]octane (such as 2-azaspiro[3.4]octane), aziridine, azetidine, diazetidine, azetidinone, pyrrolidine, pyrroline, pyrazolidine, imidazolidine, pyrazoline, imidazoline, pyrazole, succinimide, oxazolidone, morpholine, thiomorpholine, indoline, azaadamantane, azepine, and diazepine. In some embodiments, C is selected from piperidine, piperazine, diazepane (such as 1,4-diazepane), diazaspiro[3.3]heptane (such as 2,6-diazaspiro[3.3]heptane), diazaspiro[3.4]octane (such as 2,6-diazaspiro[3.4]octane), azaspiro[3.3]heptane (such as 2-azaspiro[3.3]heptane), azaspiro[3.4]octane (such as 2-azaspiro[3.4]octane), In some embodiments, C is selected from piperidine, piperazine, diazepane (such as 1,4-diazepane), and azetidine.

Typically, when A is

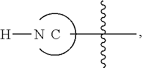

the secondary amino group that makes up ring C reacts with the moiety suitable for reacting with A.

In some embodiments, ring C comprises a basic amine group (i.e., a basic amino moiety), or ring C is a basic amine, as described above.

In some embodiments, ring C comprises no more than two nitrogen atoms.

In some embodiments, ring C is a 5- to 8-membered monocyclic ring or a bicyclic spiro moiety comprising 4- to 6-membered rings, each optionally substituted with one or more selected from halo, C₁₋₆alkyl, C₁₋₆haloalkyl, C₁₋₆alkoxy, hydroxy, aryl, heteroaryl, and C₁₋₆haloalkoxy. In some embodiments, ring C is a 5- to 8-membered monocyclic ring selected from piperidine, piperazine, diazepane (such as 1,4-diazepane), pyrrolidine, pyrroline, pyrazolidine, imidazolidine, pyrazoline, imidazoline, pyrazole, succinimide, oxazolidone, morpholine, thiomorpholine, indoline, azepine, and diazepine, typically piperidine, piperazine, and diazepane (such as 1,4-diazepane). In some embodiments, ring C is a bicyclic spiro moiety comprising 4- to 6-membered rings, such as an azaspiroalkanyl or a diazaspiroalkanyl, for example, 2H-2-azaspiro[3.3]heptanyl, 2H-2,6-diazaspiro[3.3]heptanyl, 2H-2-azaspiro[3.4]octanyl, 2H-2,6-diazaspiro[3.4]octanyl, 2H-2-azaspiro[3.4]octanyl, and 2H-2,7-diazaspiro[3.4]octanyl. The abovementioned bicyclic spiro moieties are depicted in formula (IIIc) to (IIIe) below. Typically, where ring C is a bicyclic spiro moiety, it is 2H-2,6-diazaspiro[3.3]heptanyl or 2H-2,7-diazaspiro[3.4]octanyl.

In some embodiments, ring C is selected from formulae (IIIa) to (IIIe):

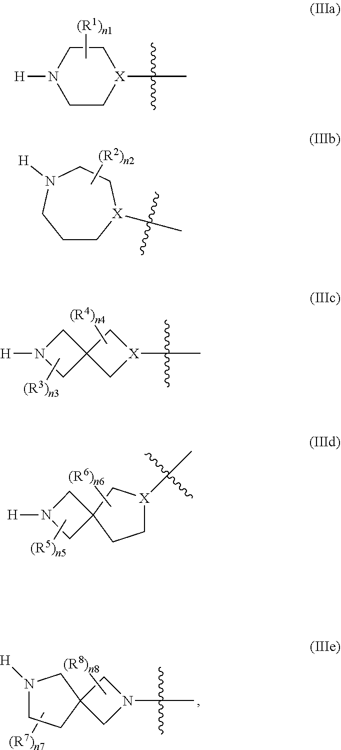

wherein:
   X is N or CH;
   each R¹ to R⁸ is independently selected from halo, C₁₋₆alkyl, C₁₋₆haloalkyl, C₁₋₆alkoxy, hydroxy, aryl, heteroaryl, and C₁₋₆haloalkoxy;
   n1 is 0 to 4;
   n2 is 0 to 5;
   each n3, n4, n5 and n8 is independently selected from 0 to 2; and
   each n6 and n7 is independently selected from 0 to 3.

In some embodiments, ring C is selected from formulae (IIIb), (IIIc), (IIId), and (IIIe). In some embodiments, ring C is selected from formulae (IIIc), (IIId), and (IIIe). In some embodiments, each n3, n4, n5, n6, and n7 is 0. Typically, X is N.

In some embodiments, when C is formula (IIIa) and X is N,

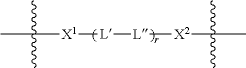

is not ethylene (i.e. is not —CH₂CH₂—).

In some embodiments, when C is formula (IIIa),

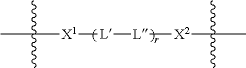

is not ethylene.

Several examples of nucleofuges are described above. In some embodiments, the nucleofuge is selected from sulfonium (such as dimethylsulfonium or diphenylsulfonium), sulfonate (such as 4-methylphenyl-1-sulfonate (toluenesulfonate, or “tosylate”), 4-bromophenyl-1-sulfonate (or “brosylate”), 4-nitrophenyl-1-sulfonate (or “nosylate”), methanesulfonate (or “mesylate”), trifluoromethanesulfonate (or “triflate”), 2,2,2-trifluoroethyl-1-sulfonate (or “tresylate”), 5-(dimethylamino)naphthalene-1-sulfonate (or “dansylate”)), or halo (such as iodo, bromo, and chloro). Typically, the nucleofuge is sulfonate or halo. Typically, wherein the nucleofuge is sulfonate, the sulfonate is selected from mesylate, tosylate, and triflate, more typically mesylate. Typically, wherein the nucleofuge is halo, the halo is selected from bromo and chloro, more typically bromo.

In some embodiments, X¹, X², or L″ is absent. In some embodiments, any two of X¹, X², and L″ is absent. In some embodiments, X¹, X², and L″ are absent.

In some embodiments, each L′ is independently selected from the group consisting of O(CH₂)ₜ, CH₂, phenylene and pyridinene, each optionally substituted with one or more substituents selected from halo, C₁₋₆alkyl, C₁₋₆haloalkyl, C₁₋₆alkoxy, hydroxy, aryl, heteroaryl, and C₁₋₆haloalkoxy.

In some embodiments,

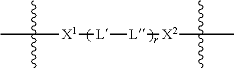

is selected from formulae (IVa) to (IVf):

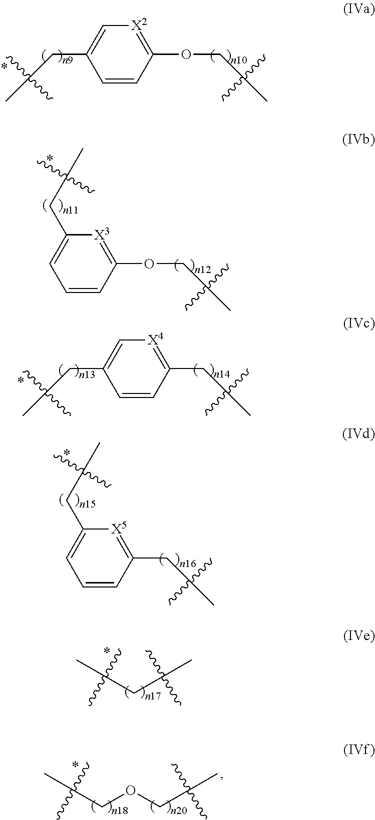

wherein:
   each X² to X⁵ is independently selected from N and CH;
   each n9 to n16, n18 and n20 is independently selected from 0 to 10; and
   each n17 and n19 is independently selected from 1 to 10.

For the avoidance of doubt, the wavy lines indicate positions of attachment. Typically, the wavy line indicated by an asterisk is connected to A.

In some embodiments, when C is formula (IIIa),

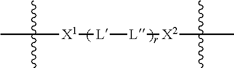

r is selected from (IVa) to (IVd), and (IVf).

In some embodiments, each n9 to n16, n18 and n20 0 to 5. In some embodiments, each n17 and n19 is independently selected from 0 to 8. In some embodiments, each n17 and n19 is independently selected from 0 to 5.

In some embodiments, n9, n11, n13, and n15 are independently selected from 0 to 5. In some embodiments, they are independently selected from 1 to 3. Typically, they are 1.

In some embodiments, n10 and n12 are independently selected from 0 to 5. In some embodiments, they are selected from 1 to 3.

In some embodiments, n17 is selected from 3 to 8. Typically, n17 is selected from 3 to 5.

In some embodiments, n19 is selected from 1 or 2. In some embodiments, n18 is 2 and n20 is 1.

In some embodiments, X² and/or X³ is CH. In some embodiments, X⁴ and/or X⁵ is N.

In some embodiments, the linker is selected from (Va) to (Vp):

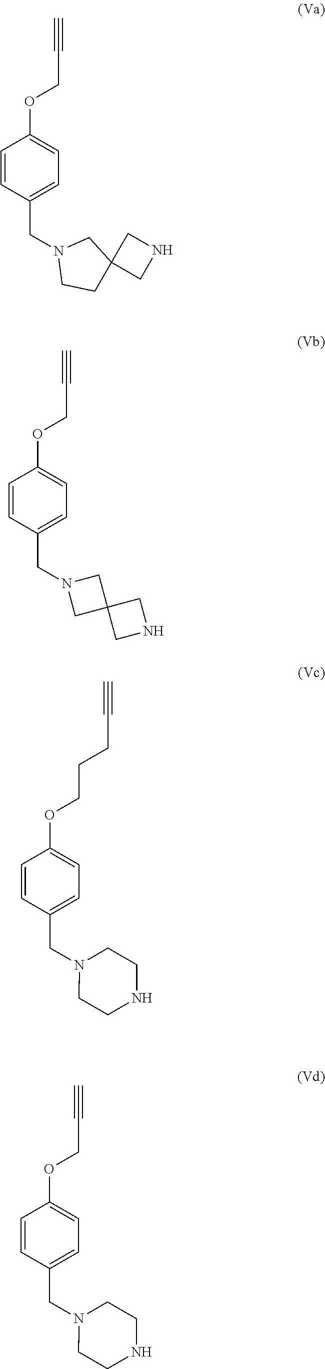

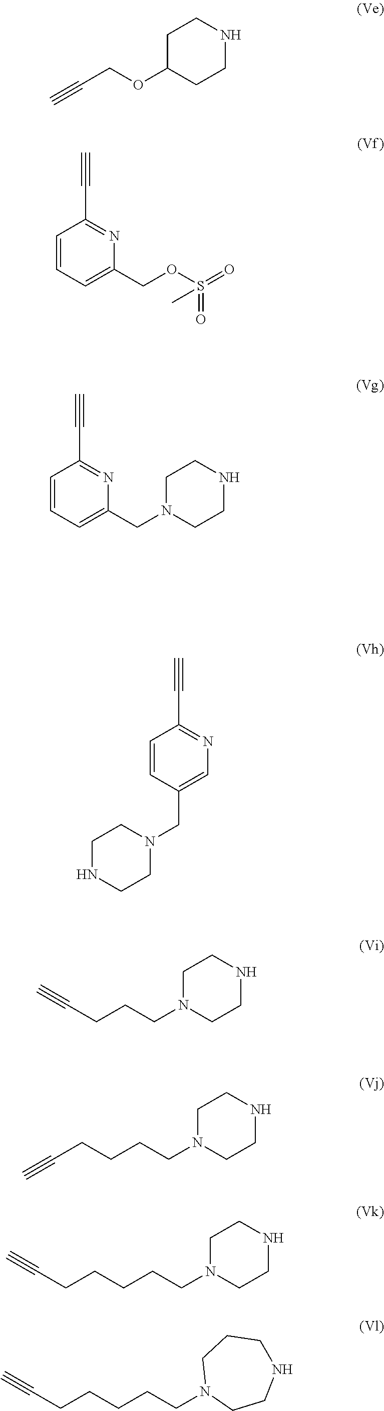

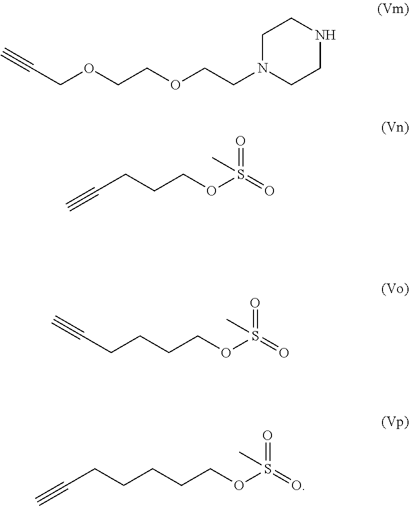

In some embodiments, when A or B is

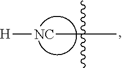

one of the two molecules comprises: (a) a nucleofuge such as a sulfonium, sulfonate or halo moiety; (b) a heteroaryl nucleofuge or aryl nucleofuge capable of undergoing: a C—N cross-coupling reaction with the linker; and/or a nucleophilic aromatic substitution reaction with the linker; in order to displace the nucleofuge; or (c) a carboxylic acid, an acyl chloride, a sulfonyl chloride, or an activated carboxylic acid, such as an N-hydroxysuccinimide ester.
Typically, when A or B is

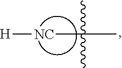

one of the two molecules comprises: (a) a nucleofuge such as a sulfonium, sulfonate or halo moiety. In some embodiments, when one of the two molecules comprises a nucleofuge, the nucleofuge is typically sulfonate or halo. In some embodiments, the sulfonate is selected from 4-methylphenyl-1-sulfonate (toluenesulfonate, or “tosylate”), 4-bromophenyl-1-sulfonate (or “brosylate”), 4-nitrophenyl-1-sulfonate (or “nosylate”), methanesulfonate (or “mesylate”), trifluoromethanesulfonate (or “triflate”), 2,2,2-trifluoroethyl-1-sulfonate (or “tresylate”), 5-(dimethylamino)naphthalene-1-sulfonate (or “dansylate”)), or halo (such as iodo, bromo, and chloro), typically mesylate. In some embodiments, halo is bromo. In some embodiments, when one of the two molecules comprises a heteroaryl nucleofuge or aryl nucleofuge capable of undergoing a C—N cross-coupling reaction with the linker, the nucleofuge is selected from iodide, bromide, chloride, or sulfonate, such as mesylate, tosylate, or triflate. Typically, the nucleofuge is selected from tosylate, iodide, or bromide.

Where the C—N cross-coupling reaction is catalysed by a palladium catalyst, it is often referred to as a Buchwald-Hartwig coupling. Common reagents and reaction conditions for relevant C—N couplings may be found in the literature, for example: A Buchwald-Hartwig Protocol to Enable Rapid Linker Exploration of Cereblon E3-Ligase PROTACs, Hayhow et al., Chem. Eur. J., 2020, 26, 16818-16823.

In some embodiments, when one of the two molecules comprises a heteroaryl nucleofuge or aryl nucleofuge capable of undergoing a nucleophilic aromatic substitution reaction (S_{N}Ar reaction) with the linker, the nucleofuge is selected from bromide, chloride, fluoride, and sulfonate, such as mesylate, triflate, and tosylate. Typically, the nucleofuge is fluoride. Common reagents and reaction conditions for relevant S_{N}Ar reactions may be found in the literature, for example: Rapid synthesis of pomalidomide-conjugates for the development of protein degrader libraries, Brownsey et al., Chem. Sci., 2021, 12, 4519.

In some embodiments, when one of the two molecules is an activated carboxylic acid, the activated carboxylic acid is selected from an N-hydroxysuccinimide ester, acid anhydride, ester, carbodiimide, and nitrophenyl ester. Typically, the activated carboxylic acid is an N-hydroxysuccinimide ester. Activated carboxylic acids are often used to form amide bonds with amines, as they are more reactive than carboxylic acids. Common reagents and reaction conditions for relevant amide coupling reactions may be found in the literature, for example: Hendrick et al., in ACS Med. Chem. Lett., 2022, 13, 1182.

In some embodiments, when A or B is ethynyl, one of the two molecules comprises an azide or a nucleofuge. In some embodiments, when A or B is a nucleofuge, such as a sulfonium, sulfonate or a halo moiety, one of the two molecules comprises a nucleophile capable of undergoing nucleophilic substitution with the linker, in order to displace the nucleofuge. In some embodiments, the nucleofuge is selected from sulfonium (such as dimethylsulfonium or diphenylsulfonium), sulfonate or halo. Typically, the nucleofuge is sulfonate or halo. In some embodiments, the sulfonate is selected from 4-methylphenyl-1-sulfonate (toluenesulfonate, or “tosylate”), 4-bromophenyl-1-sulfonate (or “brosylate”), 4-nitrophenyl-1-sulfonate (or “nosylate”), methanesulfonate (or “mesylate”), trifluoromethanesulfonate (or “triflate”), 2,2,2-trifluoroethyl-1-sulfonate (or “tresylate”), 5-(dimethylamino)naphthalene-1-sulfonate (or “dansylate”)), or halo (such as iodo, bromo, and chloro), typically mesylate. In some embodiments, halo is bromo. In some embodiments, the nucleophile is selected from thiol, hydroxy, and amino, typically thiol.

In some embodiments, one or both of the molecules comprises/comprise binders for biological molecules. The term “binders” is defined above, and is used interchangeably with “ligand”. The skilled person will recognise that the methods and linkers disclosed herein are of wide utility and not necessarily limited to any specific binder, or specific class of binder.

Nonetheless, in some embodiments, one of the molecules comprises an E3 ubiquitin ligase binder and the other comprises a target protein binder, such that the bifunctional compound is a proteolysis targeting chimera, or “PROTAC”. The skilled person is aware of many E3 ubiquitin ligase binders, such as those depicted in WO2021113557 A1, WO2020051564 A1, WO2020132561 A1, and E3 Ligase Ligands in Successful PROTACs: An Overview of Syntheses and Linker Attachment Points, Bricelj et al., Front. Chem., 2021, 9:707317, which are incorporated herein by reference.

An E3 ubiquitin ligase (sometimes just “ubiquitin ligase”, or “E3 ligase”) is a ligase enzyme that combines with a ubiquitin-containing E2 ubiquitin-conjugating enzyme, recognises the target protein that is to be ubiquitinated, and causes the attachment of ubiquitin to a lysine on the target protein via an isopeptide bond. An E3 ubiquitin ligase targets specific protein substrates for degradation by the proteasome. In general, the ubiquitin ligase is involved in poly-ubiquitination: a second ubiquitin is attached to the first, a third is attached to the second, and so forth. Poly-ubiquitination marks proteins for degradation by the proteasome. Hence, recruitment of an E3 ligase to a target protein can cause the target protein's degradation.

A target protein is any protein of interest, that for scientific, medicinal, and/or therapeutic purposes, degradation is sought. A target protein may be a tagged protein, such as a protein tagged with BromoTag, dTAG, SNAP-tag, CLIP-tag, SpyCatcher, HaloTAG, and any other suitable protein tag known to the skilled person.

In some embodiments, the E3 ubiquitin ligase binder may be selected from a cereblon E3 ubiquitin ligase (CRBN) binder, an IAP E3 ubiquitin ligase binder, a Von Hippel-Lindau E3 ubiquitin ligase (VHL) binder, DDB1, CUL4 Associated Factor 1 (DCAF1), kelch domain-containing protein 2 (KLHDC2), and a mouse double minute 2 homologue (MDM2) ubiquitin ligase binder. Some examples of ligands capable of binding to an E3 ubiquitin ligase (“E3 ubiquitin ligase binders”) include but are not limited to the following and their derivatives: thalidomide, lenalidomide, pomalidomide, CC-885, eragidomide, iberdomide, cemsidomide, golcadomide, phenyl glutarimide, phenyl aminoglutarimide, phenyl dihydrouracil, ALV2, VH032, VH-298, TD-106, LCL161, VHL-IN-1, hydroxyproline, and VL-269.

In some embodiments, the E3 ubiquitin ligase binder comprises:

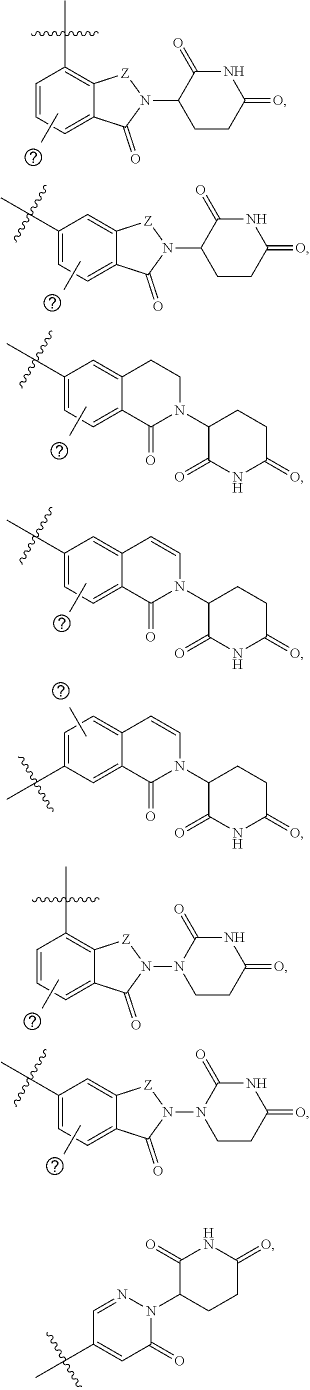

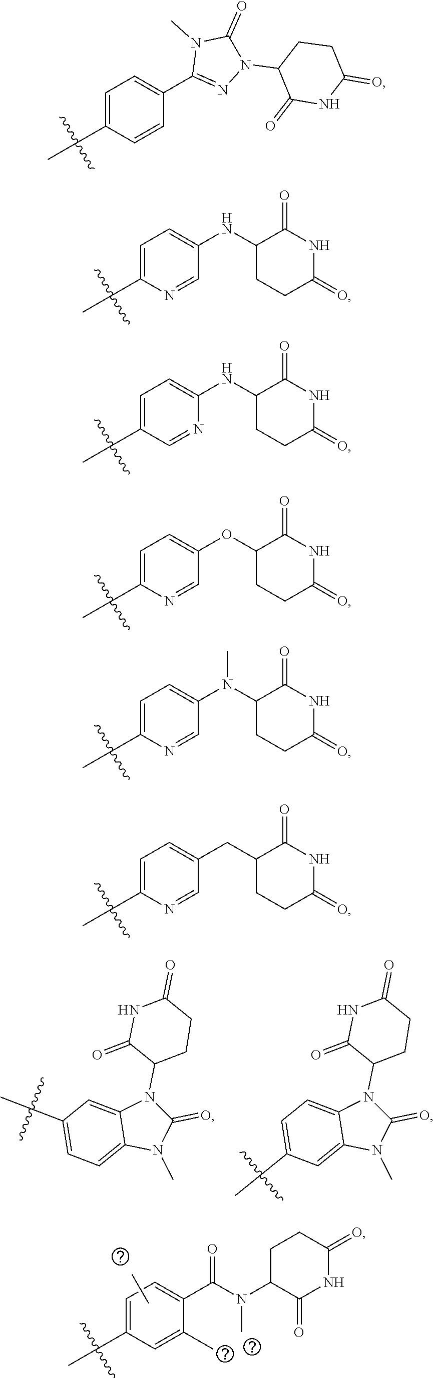

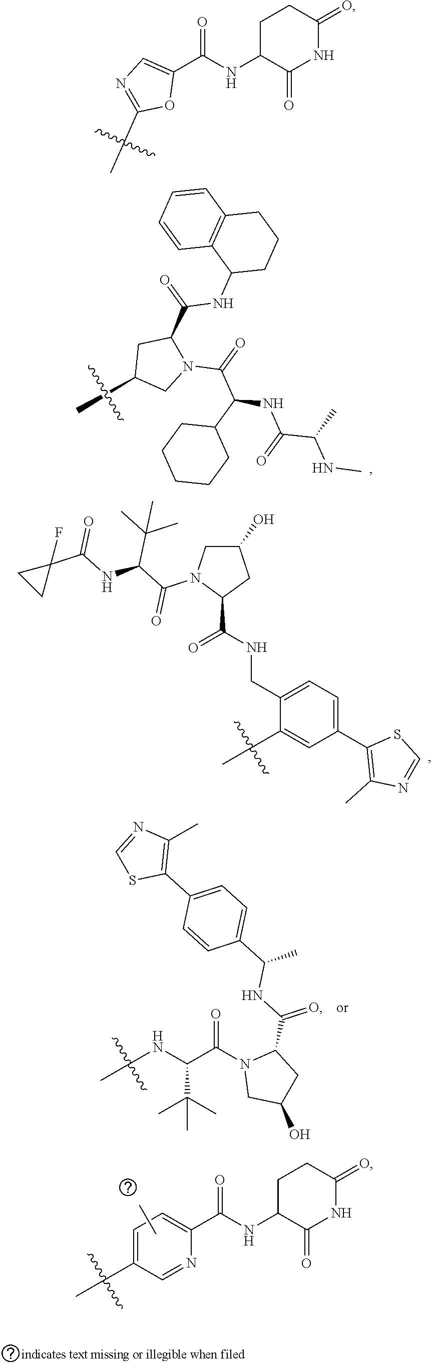

wherein each R² is independently selected from halo, nitrile, or C₁₋₄alkyl (such as methyl, ethyl, n-propyl, iso-propyl, n-butyl, sec-butyl, iso-butyl, and tert-butyl), and wherein each C₁₋₄alkyl is optionally and independently substituted with up to three instances of halo, nitrile, carboxy, carboxamide, amino, or trifluoromethyl; each Z is —C(R^{A})₂— or —C(O)—; each R^{a} is independently H or C₁₋₄alkyl; q is 0, 1, or 2; and the wavy line indicates the position of attachment to the moiety for reacting with one of the two orthogonally reactive moieties.

In some embodiments, one of the molecules is of formula (VIIa):

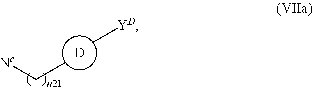

wherein:
   N^{c} is a nucleofuge;
   n21 is an integer selected from 1 to 10;
   D is an aromatic or heteroaromatic ring, optionally substituted with halo, C₁₋₆alkyl, C₁₋₆haloalkyl, C₁₋₆alkoxy, and C₁₋₆haloalkoxy; and

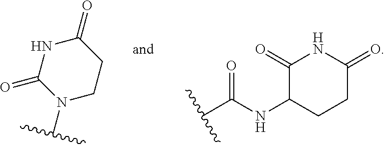 [SMILES: *N1CCC(=O)NC1=O ; *C(=O)NC1CCC(=O)NC1=O]

Y^{D} is selected from and H.

In some embodiments, the nucleofuge is selected from sulfonium (such as dimethylsulfonium or diphenylsulfonium), sulfonate (such as 4-methylphenyl-1-sulfonate (toluenesulfonate, or “tosylate”), 4-bromophenyl-1-sulfonate (or “brosylate”), 4-nitrophenyl-1-sulfonate (or “nosylate”), methanesulfonate (or “mesylate”), trifluoromethanesulfonate (or “triflate”), 2,2,2-trifluoroethyl-1-sulfonate (or “tresylate”), 5-(dimethylamino)naphthalene-1-sulfonate (or “dansylate”)), or halo (such as iodo, bromo, and chloro). Typically, wherein the nucleofuge is sulfonate, the sulfonate is selected from mesylate, tosylate, and triflate, more typically mesylate. Typically, wherein the nucleofuge is halo, the halo is selected from bromo and chloro, more typically bromo.

In some embodiments, n21 is selected from 1 to 5, typically 1.

In some embodiments, D is a 6-membered aromatic or heteroaromatic ring, such as benzene, pyridine, pyrimidine, pyrazine, or pyridazine, typically benzene or pyridine.

In some embodiments, D is a fused bicyclic aromatic or heteroaromatic ring, such as indole, isoindole, indazole, benzamidiazole, azaindole, benzofuran, benzothiophene, benzoisoxazole, benzoxazole, and benzothiazole, typically indazole. In some embodiments wherein D is indazole, the indazole is substituted with C₁₋₆alkyl or C₁₋₆haloalkyl, typically on N1 with methyl, ethyl, or trifluoromethyl, most typically methyl. In some embodiments, D is selected from

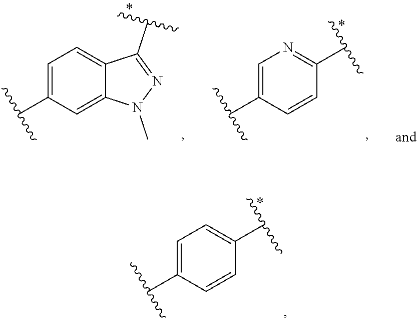 [SMILES: *c1ccc2c(*)nn(C)c2c1 ; *c1ccc(*)nc1 ; *c1ccc(*)cc1]

wherein the wavy lines indicate positions of attachment. Typically, the wavy line indicated by an asterisk is connected to Y^{D}.

In some embodiments, one of the molecules is selected from formulae (VIIb) to (VIIf):

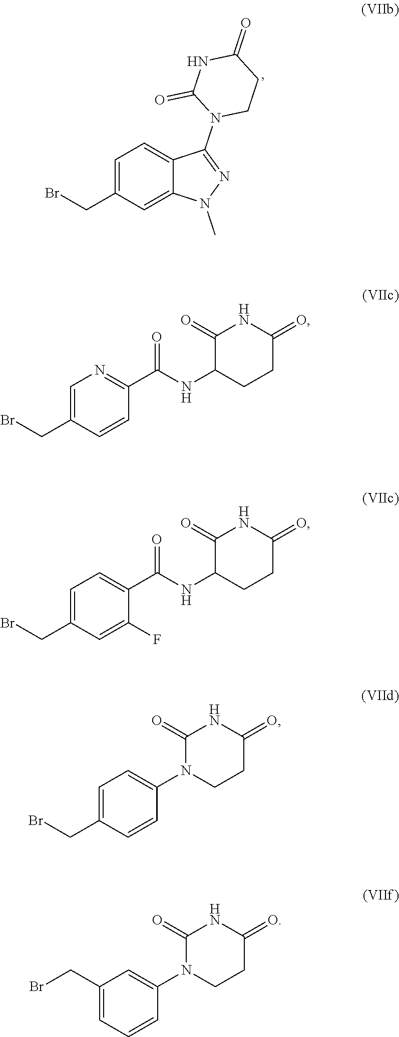 [SMILES: Cn1nc(N2CCC(=O)NC2=O)c2ccc(CBr)cc21 ; O=C1CCC(NC(=O)c2ccc(CBr)cn2)C(=O)N1 ; O=C1CCC(NC(=O)c2ccc(CBr)cc2F)C(=O)N1 ; O=C1CCN(c2ccc(CBr)cc2)C(=O)N1 ; O=C1CCN(c2cccc(CBr)c2)C(=O)N1]

In some embodiments, one of the molecules is:

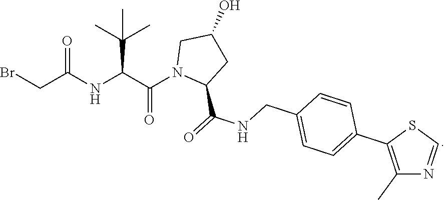 [SMILES: Cc1ncsc1-c1ccc(CNC(=O)[C@@H]2C[C@@H](O)CN2C(=O)[C@@H](NC(=O)CBr)C(C)(C)C)cc1]

In some embodiments, the target protein binder is selected from a kinase inhibitor, a phosphatase inhibitor, a binder of a bromodomain-containing protein such as but not limited to BET or SMARCA, a GTPase, an HDM2/MDM2 inhibitor, a heat shock protein 90 inhibitor, an HDAC inhibitor, a solute carrier and a human lysine methyltransferase inhibitor.

The skilled person will recognise that there are many possible target proteins compatible with the methods described herein, such as those described in: The PROTAC table genome, Schneider et al., Nature Reviews Drug Discovery, 2021, 20, 789-797; and PROTAC-DB 2.0: an updated database of PROTACs, Weng et al., Nucleic Acids Research, 2023, 51, D1367-D1372.

Examples of kinase inhibitors may be found in Jones et al., Small-Molecule Kinase Downrequlators (2017, Cell Chem. Biol., 25: 30-35). Further examples include but are not limited to the following and their derivatives: erlotinib, sunitinib, sorafenib, dasatanib, lapatanib, U09-CX-5279, vandetanib, vemurafenib, imatinib, and pazopanib.

Examples of phosphatase inhibitors include, but are not limited to the following and their derivatives: aminophylline, pentoxifylline, theobromine, theophylline, vinpocetine, EHNA (erythro-9-(2-hydroxy-3-nonyl)adenine), anagrelide, enoximone milrinone, levosimendon, mesembrine, ibudilast, piclamilast, luteolin, drotaverine, roflumilast, icariin, 4-methylpiperazine, and pyrazolo pyrimidin-7-1.

The BET (bromo- and extra-terminal domain) family of proteins comprise bromodomains, of which many ligands are known to bind to. Examples of ligands that bind to BET proteins include the following and their derivatives: JQ1, GSK525762A (I-BET-762), OTX015, BMS-986158, TEN-010, CPI-0610, INCB54329, BAY1238097, FT-1101, ABBV-075, BI 894999, GS-5829, GSK1210151A (I-BET-151), CPI-203, RVX-208, XD46, MS436, PFI-1, RVX2135, ZEN3365, XD14, ARV-771, MZ-1, PLX5117, EPI 1313 and EPI 1336.

ATP-dependent chromatin re-modellers SMARCA2 and SMARCA4 are components of the SWI/SWF (SWitch/Sucrose Non-Fermentable) complex, which are involved in the regulation of gene expression by altering chromatin structure. SMARCA2 and SMARCA4 play important roles in cell differentiation and development, thus they are interesting targets for therapeutic applications, especially cancer. Examples of SMARCA2 and SMARCA4 inhibitors may be found in: GNE-064: A Potent, Selective, and Orally Bioavailable Chemical Probe for the Bromodomains of SMARCA2 and SMARCA4 and the Fifth Bromodomain of PBRM1, Taylor et al., J. Med. Chem., 2022, 65, 11177-11186; Selective PROTAC-mediated degradation of SMARCA2 is efficacious in SMARCA4 mutant cancers, Cantley et al., Nat. Commun., 2022, 13, 6814; and Proteolysis Targeting Chimera (PROTACS) as Degraders of SMARCA2 and/or SMARCA4, WO 2020/078933 A1.

Ras GTPases are a family of proteins involved in transmitting signals within cells, involved in cell growth, differentiation, and survival, thus are interesting targets for therapeutic applications, especially cancer. Examples of Ras GTPase inhibitors may be found in: RAS degraders: The new frontier for RAS-driven cancers, Escher et al., Molecular Therapy, 2023, 31, 1904-1919; and Progress in RAS-targeted therapeutic strategies: From small molecule inhibitors to proteolysis targeting chimeras, Lu et al., Medicinal Research Reviews, 2023, 44, 812-832.

The solute carrier family (SLC) is a group of membrane transport proteins, involved in facilitated diffusion or active transport of a wide variety of substances. Mutations or dysfunctions in SLC proteins may be associated with numerous diseases, such as cystic fibrosis, and neurological disorders. Examples of solute carrier inhibitors may be found in: Targeted Degradation of SLC Transporters Reveals Amenability of Multi-Pass Transmembrane Proteins to Ligand-Induced Proteolysis, Bensimon et al., Cell Chem. Biol., 2020, 27, 728-739; and Targeting SLC transporters: small molecules as modulators and therapeutic opportunities, Schlessinger et al., Trends in Biochemical Sciences, 2023, 48, 801-814.

Examples of HDM2/MDM2 inhibitors include, but are not limited to the following and their derivatives: the HDM2/MDM2 inhibitors identified in Vassilev, et al., In vivo activation of the p53 pathway by small-molecule antagonists of MDM2, (2004, Science, 303844-848), and Schneekloth, et al., Targeted intracellular protein degradation induced by a small molecule: En route to chemical proteomics, (2008, Biorg. Med. Chem. Lett., 18:5904-5908), including (or additionally) the compounds nutlin-3, nutlin-2, and nutlin-1 (derivatized).

Examples of Heat Shock Protein 90 (HSP90) inhibitors include, but are not limited to the following and their derivatives: Vallee, et al., Tricyclic Series of Heat Shock Protein 90 (HSP90) Inhibitors Part I: Discovery of Tricyclic Imidazo[4,5-C]Pyridines as Potent Inhibitors of the HSP90 Molecular Chaperone (2011, J. Med. Chem., 54:7206); Brough, et al., 4,5-Diarylisoxazole HSP90 Chaperone Inhibitors: Potential Therapeutic Agents for the Treatment of Cancer, (2008, J. Med. Chem., 51:196); and Wright, et al., Structure-Activity Relationships in Purine-Based Inhibitor Binding to HSP90 Isoforms, (2004 Jun., Chem. Biol. 11 (6): 775-85).

Examples of HDAC inhibitors include, but are not limited to the following and their derivatives: Finnin, M. S. et al. Structures of Histone Deacetylase Homologue Bound to the TSA and SAHA Inhibitors. (1999, Nature, 40:188-193); and compounds as defined by formula (I) of PCT WO0222577 (the entire contents of which are incorporated herein by reference).

Examples of Human Lysine Methyltransferase inhibitors include, but are not limited to the following and their derivatives: Chang et al. Structural Basis for G9a-Like protein Lysine Methyltransferase Inhibition by BIX-1294, (2009, Nat. Struct. Biol., 16(3):312); and Liu, F. et al, Discovery of a 2,4-Diamino-7-aminoalkoxyquinazoline as a Potent and Selective Inhibitor of Histone Methyltransferase G9a., (2009, J. Med. Chem. 52(24):7950).

In some embodiments, the molecule is a ligand for GSK3, as described in Liang et al., Discovery of a Highly Selective Glycogen Synthase Kinase-3 Inhibitor (PF-04802367) That Modulates Tau Phosphorylation in the Brain: Translation for PET Neuroimaging, Angew. Chem. Int. Ed., 2106, 55, 9601. In some embodiments, the molecule is:

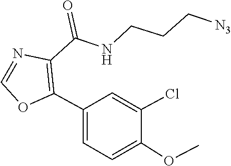 [SMILES: COc1ccc(-c2ocnc2C(=O)NCCCN=[N+]=[N-])cc1Cl]

In some embodiments, where the bifunctional compound is a proteolysis targeting chimera, the method further comprises contacting the resultant mixture with a target protein and/or a cell. In such embodiments, the method may comprise analysing the resultant mixture for formation of a bifunctional compound, optionally where the analysing shows formation of a proteolysis targeting chimera. The target protein may be any target protein described above, such as a kinase inhibitor, a phosphatase inhibitor, a binder of a bromodomain-containing protein such as but not limited to BET or SMARCA, a GTPase, an HDM2/MDM2 inhibitor, a heat shock protein 90 inhibitor, an HDAC inhibitor, a solute carrier and a human lysine methyltransferase inhibitor. In some embodiments, the target protein is GSK3β. The cell may be any cell suitable of expressing the target protein. The skilled person will recognise that there are many suitable cell lines to carry out the methods disclosed herein, and that the methods are not limited to any particular one. Nonetheless, in some embodiments, the cell is HEK293.

### Linkers

As described above, in a second aspect there is provided a linker of formula (1):

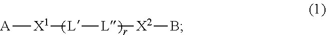

wherein:
A is

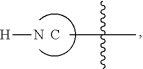

wherein ring C is a bicyclic spiro moiety comprising 4- to 6-membered aliphatic N-heterocyclic rings and optionally substituted with one or more selected from halo, C₁₋₆alkyl, C₁₋₆haloalkyl, C₁₋₆alkoxy, hydroxy, aryl, heteroaryl, and C₁₋₆haloalkoxy, and B is ethynyl or a nucleofuge;
each X¹ and X² is optionally present and is any one selected from the group consisting of O(CH₂)ₛ and N(C₁₋₆alkyl)(CH₂)₁;
each L′ is independently selected from the group consisting of O(CH₂)ₜ, CH₂, heterocyclylene, arylene, and cycloalkylene, each optionally substituted with one or more substituents selected from halo, C₁₋₆alkyl, C₁₋₆haloalkyl, C₁₋₆alkoxy, hydroxy, aryl, heteroaryl, and C₁₋₆haloalkoxy;
L″ is optionally present and is selected from —O— and —N(C₁₋₆alkyl)-; and
r is an integer from 1 to 20, s is an integer from 0 to 4, and t is an integer from 1 to 4.

As described above, in some embodiments, A is

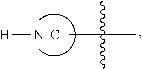

wherein ring C is a bicyclic spiro moiety comprising 4- to 6-membered aliphatic N-heterocyclic rings, such as an azaspiroalkanyl or a diazaspiroalkanyl, for example, 2H-2-azaspiro[3.3]heptanyl, 2H-2,6-diazaspiro[3.3]heptanyl, 2H-2-azaspiro[3.4]octanyl, 2H-2,6-diazaspiro[3.4]octanyl, 2H-2-azaspiro[3.4]octanyl, and 2H-2,7-diazaspiro[3.4]octanyl. The abovementioned bicyclic spiro moieties are depicted in formula (IIIc) to (IIIe) above. Typically, where ring C is a bicyclic spiro moiety, it is 2H-2,6-diazaspiro[3.3]heptanyl or 2H-2,7-diazaspiro[3.4]octanyl.

In some embodiments, ring C comprises a basic amine group (i.e., a basic amino moiety), or ring C is a basic amine, as described above.

In some embodiments, A comprises no more than two nitrogen atoms.

In some embodiments, A is selected from formulae (2a) to (2c):

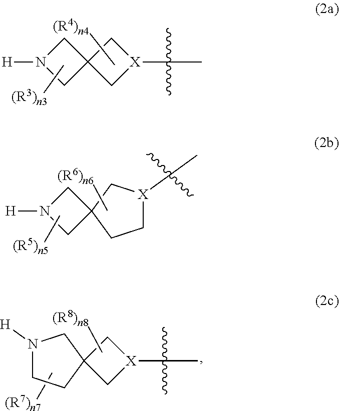

wherein:
   X is N or CH;
   each R³ to R⁸ is independently selected from halo, C₁₋₆alkyl, C₁₋₆haloalkyl, C₁₋₆alkoxy, hydroxy, aryl, heteroaryl, and C₁₋₆haloalkoxy;
   each n3, n4, n5 and n8 is independently selected from 0 to 2; and
   each n6 and n7 is independently selected from 0 to 3.

In some embodiments, X is N, and each n3 to n8 is 0.

In some embodiments, the nucleofuge is selected from sulfonium (such as dimethylsulfonium or diphenylsulfonium), sulfonate (such as 4-methylphenyl-1-sulfonate (toluenesulfonate, or “tosylate”), 4-bromophenyl-1-sulfonate (or “brosylate”), 4-nitrophenyl-1-sulfonate (or “nosylate”), methanesulfonate (or “mesylate”), trifluoromethanesulfonate (or “triflate”), 2,2,2-trifluoroethyl-1-sulfonate (or “tresylate”), 5-(dimethylamino)naphthalene-1-sulfonate (or “dansylate”)), or halo (such as iodo, bromo, and chloro). Typically, the nucleofuge is sulfonate or halo. Typically, wherein the nucleofuge is sulfonate, the sulfonate is selected from mesylate, tosylate, and triflate, more typically mesylate. Typically, wherein the nucleofuge is halo, the halo is selected from bromo and chloro, more typically bromo.

In some embodiments, X¹, X², or L″ is absent. In some embodiments, any two of X¹, X², and L″ is absent. In some embodiments, X¹, X², and L″ are absent.

In some embodiments, L′ is independently selected from the group consisting of O(CH₂)ₜ, CH₂, phenylene and pyridinene, each optionally substituted with one or more substituents selected from halo, C₁₋₆alkyl, C₁₋₆haloalkyl, C₁₋₆alkoxy, hydroxy, aryl, heteroaryl, and C₁₋₆haloalkoxy.

In some embodiments,

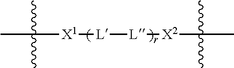

is selected from formulae (3a) to (3f):

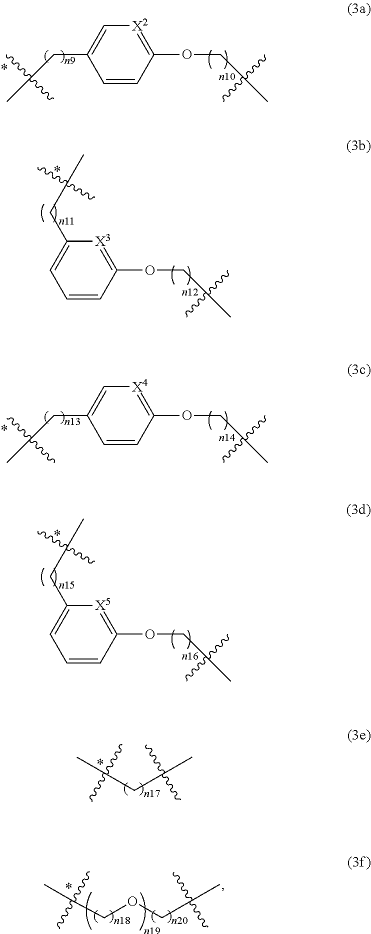

wherein:
   each X² to X⁵ is independently selected from N and CH;
   each n9 to n16, n18 and n20 is independently selected from 0 to 10; and
   each n17 and n19 is independently selected from 1 to 10.

For the avoidance of doubt, the wavy lines indicate positions of attachment. Typically, the wavy line indicated by an asterisk is connected to A.

In some embodiments, each n9 to n16, n18 and n20 0 to 5. In some embodiments, each n17 and n19 is independently selected from 0 to 8. In some embodiments, each n17 and n19 is independently selected from 0 to 5.

In some embodiments, n9, n11, n13, and n15 are independently selected from 0 to 5. In some embodiments, they are independently selected from 1 to 3. Typically, they are 1.

In some embodiments, n10 and n12 are independently selected from 0 to 5. In some embodiments, they are selected from 1 to 3.

In some embodiments, n17 is selected from 3 to 8. Typically, n17 is selected from 3 to 5.

In some embodiments, n19 is selected from 1 or 2. In some embodiments, n18 is 2 and n20 is 1.

In some embodiments, X² and/or X³ is CH. In some embodiments, X⁴ and/or X⁵ is N.

In some embodiments, the linker is of formula (4a) or (4b):

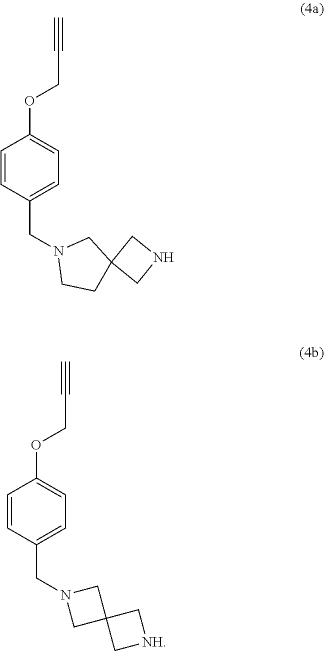 [SMILES: C#CCOc1ccc(CN2CCC3(CNC3)C2)cc1 ; C#CCOc1ccc(CN2CC3(CNC3)C2)cc1]

In another aspect, there is provided a linker of formula (5a) or (5b):

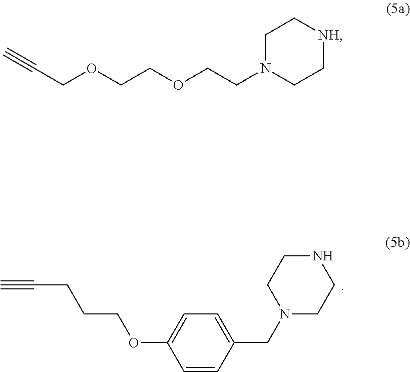 [SMILES: C#CCOCCOCCN1CCNCC1 ; C#CCCCOc1ccc(CN2CCNCC2)cc1]

### Uses/kits

As described above, in a third aspect there is provided a method of manufacturing a bifunctional compound, optionally as a one-pot method, the method comprising:
(i) contacting:
(a) a linker of the second aspect; and
(b) two molecules, one of which comprises a moiety for reacting with one of A and B, and the other of which comprises a moiety for reacting with the other of A and B;
and optionally:
(ii) analysing the resultant mixture for formation of a bifunctional compound comprising each of the two molecules linked together by the linker.

In a fourth aspect, there is provided a method of targeted protein degradation or stabilisation, the method comprising:
(i) contacting:
(a) a linker of the second aspect; and
(b) two molecules, one of which comprises a moiety for reacting with one of A and B, and the other of which comprises a moiety for reacting with the other of A and B, and wherein one of the molecules comprises a target protein binder and the other comprises an E3 ubiquitin ligase or a deubiquitinase binder;
(ii) analysing the resultant mixture for formation of a bifunctional compound comprising each of the two molecules linked together by the linker; and
(iii) contacting the bifunctional compound with the target protein.

For the avoidance of doubt, the embodiments described in relation to the second aspect of the invention apply mutatis mutandis to the embodiments of the third and fourth aspects of the invention.

The manufacture of a bifunctional compound may be a one-pot method in accordance with embodiments of the first aspect of the invention, but this is not required. The manufacture of a bifunctional compound using a linker according to the second aspect may be a conventional synthetic chemistry process with isolation and purification steps and the like; such processes are well known to the skilled person. Manufacturing processes that comprise any one or more chemical reaction steps disclosed herein but not carried out in a one-pot method are within the scope.

Targeted protein degradation (TPD) is well known in the art. TPD is a technique whereby a target protein is brought into proximity with an E3 ubiquitin ligase, such that the target protein is ubiquitinated, thus marking the target protein for degradation. TPD is a promising modality for novel therapeutic approaches to treat diseases. Examples of target protein binders and E3 ubiquitin ligase binders are described above according to the first aspect of the invention, and their use in the third or fourth aspects may be construed accordingly. In some embodiments, the targeted protein degradation comprises the degradation of GSK3p, via a GSK3p binder and a E3 ubiquitin ligase binder, such as a CRBN binder or VHL binder.

As described above, in a fifth aspect there is provided a kit comprising:
(i) a linker according to the second aspect; and
(ii) a molecule comprising a moiety for reacting with A and/or a molecule comprising a moiety for reacting with B.

In some embodiments, the kit comprises a plurality of linkers, each independently according to the second aspect. In some embodiments the kit comprises a plurality of molecules, each independently comprising a moiety for reacting with A or a moiety for reacting with B.

In some embodiments, the kit is in a plate-based format, or is amenable to plate-based methods. That is to say, in some embodiments, the linker(s) and/or the molecule(s) is/are deposited in one or more wells of a multi-well plate. Non-limiting examples of multi-well plates include 4-well, 6-well, 8-well, 12-well, 24-well, 48-well, 96-well, 384-well, and 1536-well plates. In some embodiments wherein the kit is in a plate-based format, the linker(s) and molecule(s) are deposited in the wells of the plate such that the plate comprises an array for a method of screening reagents to assess their suitability in forming a bifunctional compound. Such arrays may be capable of producing a diverse library of bifunctional compounds.

In some embodiments, the kit comprises a negative control molecule. A negative control molecule is a molecule that comprises a moiety for reacting with A or B but does not necessarily impart a function on the resultant bifunctional molecule. Rather, the negative control molecule may be included in the kit to verify that the reaction between the linker and the molecule has worked, which may be determined through analytical techniques, such as LC-MS (optionally with a UV-vis detector). In some embodiments, the negative control molecule is benzyl bromide.

In some embodiments, the kit additionally comprises a purification system. The purification system may be in a plate-based format, such that it is compatible with kits according to the fourth aspect wherein the kit is in a plate-based format or is amenable to plate-based methods. That is to say, each well of a multi-well plate-based purification system may consist of an independent purification system. Examples of purification systems include, but are not limited to, chromatography, ion-exchange, solid-phase extraction (SPE), filtration, size-exclusion chromatography (SEC), and immunoprecipitation (IP). In some embodiments, the purification system is an ion-exchange system, such as a cation exchange system. Cation exchange systems typically comprise a bead matrix solid support, often comprising cross-linked polystyrene or silica gel, wherein the solid support comprises acidic groups (such as sulfonic acids, phosphonic acids, and carboxylic acids). The acidic groups are able to bind to basic groups on a compound to be purified that has been loaded on to the solid support; impurities that do not bind may then be washed away, for example, with an organic solvent; the purified compound may then be eluted from the solid support by changing the pH of the system. An example of a cation exchange system in a plate-based format is an SCX (strong cation exchange) 96-well plate. In such embodiments (where the kit further comprises a purification system comprising an ion-exchange system), the linker comprises

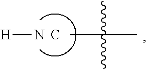

which is a basic amine group.

Where the kit comprises a plate, negative control and/or a purification system, the kit need not contain a molecule comprising a moiety for reacting with A and/or a molecule comprising a moiety for reacting with B. In other words, the kit may comprise:
(i) a linker according to the second aspect; and
(ii) a plate, negative control and/or a purification system, each of which may be as described above.

In some embodiments, the linker(s) and/or the molecule(s) are supplied in a solid form. In other embodiments, they are supplied in a liquid form, often as a solution in a suitable solvent, such as DMF, DMSO, water, or acetonitrile. The skilled person will recognise that a wide variety of solvents will be suitable, and is able to assess the suitability of a particular solvent based on the physical and chemical properties of the linker/molecule.

### Binders

As described above, in a sixth aspect, there is provided a molecule of formula (VIIa):

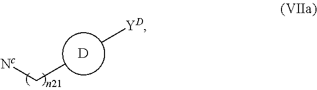

wherein:
N^{c} is a nucleofuge;
n21 is an integer selected from 1 to 10;
D is an aromatic or heteroaromatic ring, optionally substituted with halo, C₁₋₆alkyl, C₁₋₆haloalkyl, C₁₋₆alkoxy, and C₁₋₆haloalkoxy; and
Y^{D} is selected from

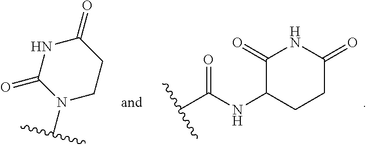 [SMILES: *N1CCC(=O)NC1=O ; *C(=O)NC1CCC(=O)NC1=O]

In some embodiments, the nucleofuge is selected from sulfonium (such as dimethylsulfonium or diphenylsulfonium), sulfonate (such as 4-methylphenyl-1-sulfonate (toluenesulfonate, or “tosylate”), 4-bromophenyl-1-sulfonate (or “brosylate”), 4-nitrophenyl-1-sulfonate (or “nosylate”), methanesulfonate (or “mesylate”), trifluoromethanesulfonate (or “triflate”), 2,2,2-trifluoroethyl-1-sulfonate (or “tresylate”), 5-(dimethylamino)naphthalene-1-sulfonate (or “dansylate”)), or halo (such as iodo, bromo, and chloro). Typically, the nucleofuge is sulfonate or halo. Typically, wherein the nucleofuge is sulfonate, the sulfonate is selected from mesylate, tosylate, and triflate, more typically mesylate. Typically, wherein the nucleofuge is halo, the halo is selected from bromo and chloro, more typically bromo.

In some embodiments, n21 is selected from 1 to 5, typically 1.

In some embodiments, D is a 6-membered aromatic or heteroaromatic ring, such as benzene, pyridine, pyrimidine, pyrazine, or pyridazine, typically benzene or pyridine.

In some embodiments, D is a fused bicyclic ring, such as indole, isoindole, indazole, benzamidiazole, azaindole, benzofuran, benzothiophene, benzoisoxazole, benzoxazole, and benzothiazole, typically indazole. In some embodiments wherein D is indazole, the indazole is substituted with C₁₋₆alkyl or C₁₋₆haloalkyl, typically on N1 with methyl, ethyl, or trifluoromethyl, most typically methyl. In some embodiments, D is selected from

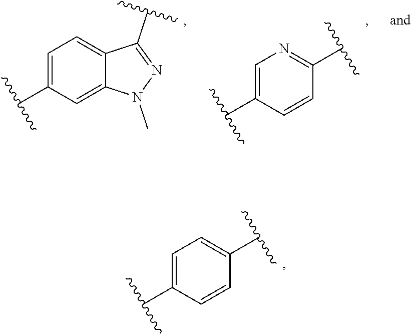 [SMILES: *c1ccc2c(*)nn(C)c2c1 ; *c1ccc(*)nc1 ; *c1ccc(*)cc1]

wherein the wavy lines indicate positions of attachment.

In some embodiments, the molecule is selected from formulae (VIIb) to (VIIf):

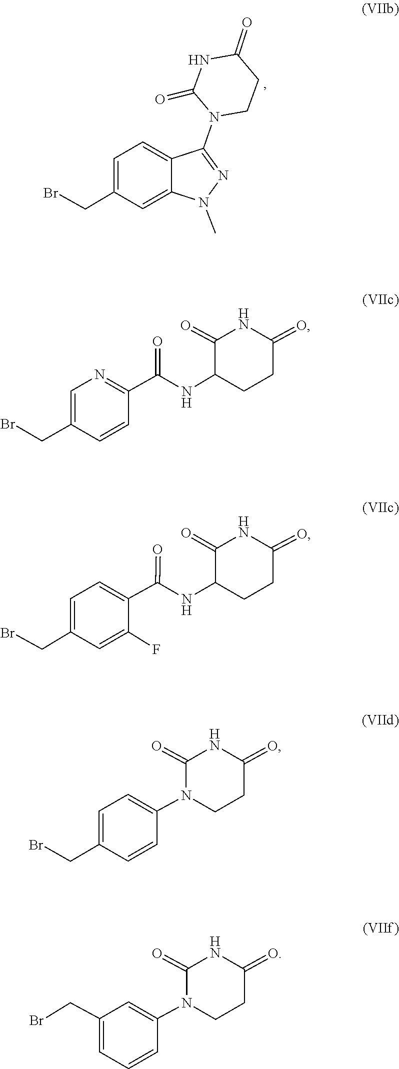 [SMILES: Cn1nc(N2CCC(=O)NC2=O)c2ccc(CBr)cc21 ; O=C1CCC(NC(=O)c2ccc(CBr)cn2)C(=O)N1 ; O=C1CCC(NC(=O)c2ccc(CBr)cc2F)C(=O)N1 ; O=C1CCN(c2ccc(CBr)cc2)C(=O)N1 ; O=C1CCN(c2cccc(CBr)c2)C(=O)N1]

The present disclosure may be further understood with reference to the clauses set out below.

### CLAUSES

Clause 1. A one-pot method comprising:
(i) contacting:
(a) a linker comprising two orthogonally reactive moieties; and
(b) two molecules, one of which comprises a moiety for reacting with one of the two orthogonally reactive moieties, and the other of which comprises a moiety for reacting with the other of the two orthogonally reactive moieties;
and optionally:
(ii) analysing the resultant mixture for formation of a bifunctional compound comprising each of the two molecules linked together by the linker.

Clause 2. The method of clause 1, wherein the method comprises analysing the resultant mixture for formation of a bifunctional compound comprising each of the two molecules linked together by the linker.

Clause 3. The method of clause 1 or clause 2, wherein the linker is of formula (I):

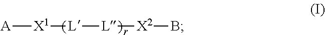

and
one of the two molecules comprises a moiety suitable for reacting with A, and the other comprises a moiety suitable for reacting with B;
wherein:
A is

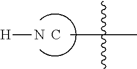

and B is ethynyl;
A is a nucleofuge and B is ethynyl; or
A is

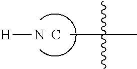

and B is a nucleofuge;
and wherein:
ring C is an aliphatic N-heterocycle optionally substituted with one or more selected from halo, C₁₋₆alkyl, C₁₋₆haloalkyl, C₁₋₆alkoxy, hydroxy, aryl, heteroaryl, and C₁₋₆haloalkoxy; each X¹ and X² is optionally present and is any one selected from the group consisting of O(CH₂)ₛ and N(C₁₋₆alkyl)(CH₂)ₛ;
each L′ is independently selected from the group consisting of O(CH₂)ₜ, CH₂, heterocyclylene, arylene, and cycloalkylene, each optionally substituted with one or more substituents selected from halo, C₁₋₆alkyl, C₁₋₆haloalkyl, C₁₋₆alkoxy, hydroxy, aryl, heteroaryl, and C₁₋₆haloalkoxy;
L″ is optionally present and is selected from —O— and —N(C₁₋₆alkyl)-;
r is an integer from 1 to 20, s is an integer from 0 to 4, and t is an integer from 1 to 4.

Clause 4. The method of clause 3, wherein the linker is of formula (IIa) or (IIb):

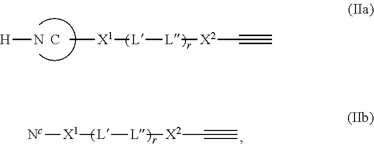

wherein:
N^{c} is the nucleofuge.

Clause 5. The method of clause 3 or clause 4, wherein ring C is a 5- to 8-membered monocyclic ring or a bicyclic spiro moiety comprising 4- to 6-membered rings, each optionally substituted with one or more selected from halo, C₁₋₆alkyl, C₁₋₆haloalkyl, C₁₋₆alkoxy, hydroxy, aryl, heteroaryl, and C₁₋₆haloalkoxy.

Clause 6. The method of any one of clauses 3 to 5, wherein ring C comprises no more than two nitrogen atoms.

Clause 7. The method of any one of clauses 3 to 6, wherein ring C is selected from formulae (IIIa) to (IIIe):

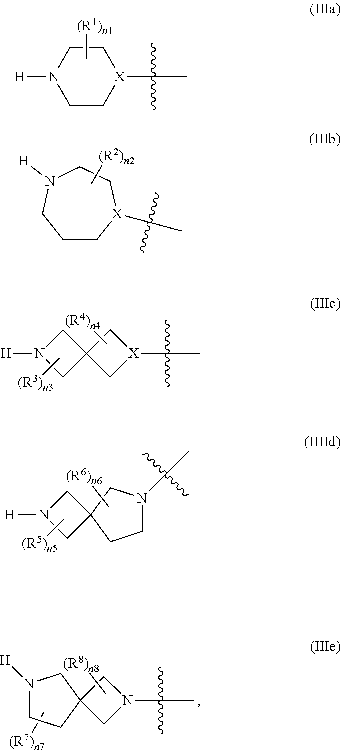

wherein:
   X is N or CH;
each R¹ to R⁸ is independently selected from halo, C₁₋₆alkyl, C₁₋₆haloalkyl, C₁₋₆alkoxy, hydroxy, aryl, heteroaryl, and C₁₋₆haloalkoxy;
n1 is 0 to 4;
n2 is 0 to 5;
each n3, n4, n5 and n8 is independently selected from 0 to 2; and each n6 and n7 is independently selected from 0 to 3.

Clause 8. The method of any one of clauses 3 to 7, wherein the nucleofuge is selected from sulfonium, sulfonate and halo.

Clause 9. The method of any one of clauses 3 to 8, wherein X¹, L″ and X² are absent.

Clause 10. The method of any one of clauses 3 to 9, wherein each L′ is independently selected from the group consisting of O(CH₂)ₜ, CH₂, phenylene and pyridinene, each optionally substituted with one or more substituents selected from halo, C₁₋₆alkyl, C₁₋₆haloalkyl, C₁₋₆alkoxy, hydroxy, aryl, heteroaryl, and C₁₋₆haloalkoxy.

Clause 11. The method of any one of clauses 3 to 10, wherein:

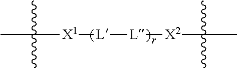

is selected from formulae (IVa) to (IVf):

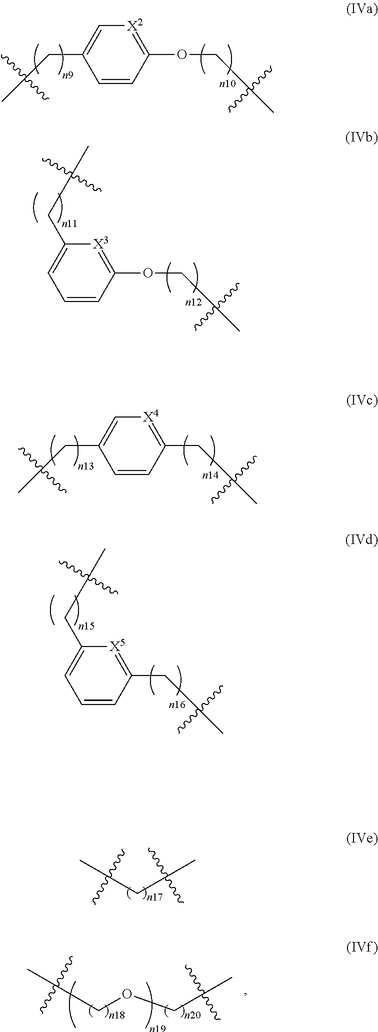

wherein:
each X² to X⁵ is independently selected from N and CH;
   each n9 to n16, n18 and n20 is independently selected from 0 to 10; and
   each n17 and n19 is independently selected from 1 to 10.

Clause 12. The method of any one of clauses 3 to 11, wherein the linker is selected from (Va) to (Vp):

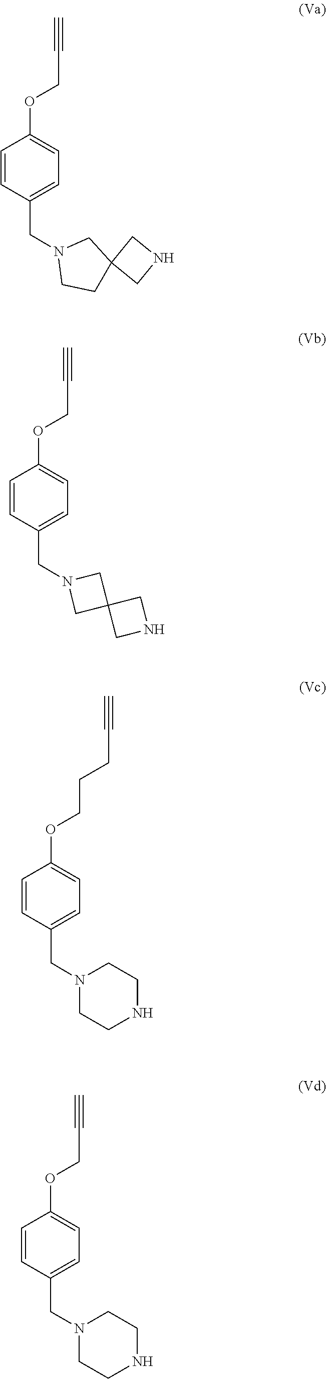 [SMILES: C#CCOc1ccc(CN2CCC3(CNC3)C2)cc1 ; C#CCOc1ccc(CN2CC3(CNC3)C2)cc1 ; C#CCCCOc1ccc(CN2CCNCC2)cc1 ; C#CCOc1ccc(CN2CCNCC2)cc1]

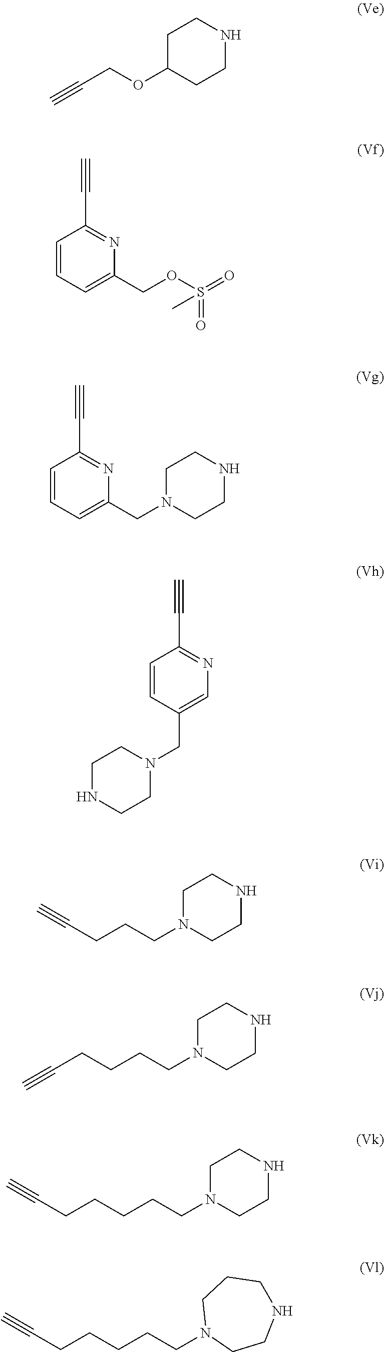

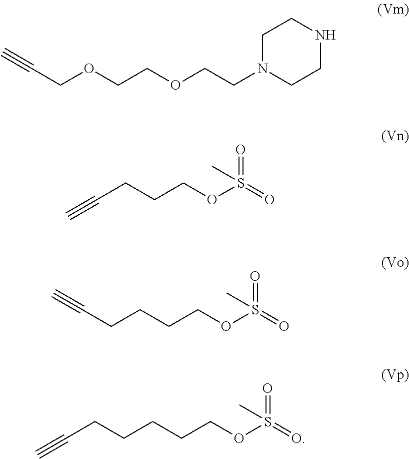

Clause 16. The method of any one of clauses 3 to 15, wherein:
(i) when A or B is

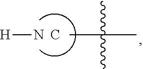

one of the two molecules comprises:
(a) a nucleofuge such as a sulfonium, sulfonate or halo moiety;
(b) a heteroaryl nucleofuge or aryl nucleofuge capable of undergoing:
a C—N cross-coupling reaction with the linker; and/or a nucleophilic aromatic substitution reaction with the linker;
in order to displace the nucleofuge; or
(c) a carboxylic acid, an acyl chloride, a sulfonyl chloride, or an activated carboxylic acid, such as an N-hydroxysuccinimide ester;
(ii) when A or B is ethynyl, one of the two molecules comprises an azide or a nucleofuge such as a sulfonate or halo moiety; and
(ii) when A or B is a nucleofuge, such as a sulfonate or a halo moiety, one of the two molecules comprises a nucleophile capable of undergoing nucleophilic substitution with the linker, in order to displace the nucleofuge.

Clause 17. The method of clause 16, wherein when A or B is

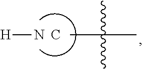

one of the two molecules comprises a nucleofuge such as a sulfonium, sulfonate or halo moiety.

Clause 18. The method of clause 16 or clause 17, wherein the nucleophile is selected from thiol, hydroxy and amino.

Clause 19. The method of any one preceding clause, wherein one or both of the molecules comprises/comprise binders for biological molecules.

Clause 20. The method of any one preceding clause, wherein one of the molecules comprises an E3 ubiquitin ligase binder and the other comprises a target protein binder, such that the bifunctional compound is a proteolysis targeting chimera.

Clause 21. The method of clause 20, wherein the E3 ubiquitin ligase binder is selected from a cereblon E3 ubiquitin ligase (CRBN) binder, an IAP E3 ubiquitin ligase binder, a Von Hippel-Lindau E3 ubiquitin ligase (VHL) binder, DDB1, CUL4 Associated Factor 1 (DCAF1), kelch domain-containing protein 2 (KLHDC2), and a mouse double minute 2 homologue (MDM2) ubiquitin ligase binder.

Clause 22. The method of clause 20 or clause 21, wherein the target protein binder is selected from a kinase inhibitor, a phosphatase inhibitor, a binder of a BET bromodomain-containing protein, an HDM2/MDM2 inhibitor, a heat shock protein 90 inhibitor, an HDAC inhibitor, and a human lysine methyltransferase inhibitor.

Clause 23. The method of any one of clauses 20 to 22, wherein, where the analysing shows formation of the proteolysis targeting chimera, the method further comprises contacting the resultant mixture with a target protein and/or a cell.

Clause 24. A linker of formula (1):

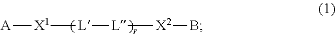

wherein:
A is

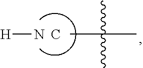

wherein ring C is a bicyclic spiro moiety comprising 4- to 6-membered aliphatic N-heterocyclic rings and optionally substituted with one or more selected from halo, C₁₋₆alkyl, C₁₋₆haloalkyl, C₁₋₆alkoxy, hydroxy, aryl, heteroaryl, and C₁₋₆haloalkoxy, and B is ethynyl or a nucleofuge;
each X¹ and X² is optionally present and is any one selected from the group consisting of O(CH₂)ₛ and N(C₁₋₆alkyl)(CH₂)ₛ;
each L′ is independently selected from the group consisting of O(CH₂)ₜ, CH₂, heterocyclylene, arylene, and cycloalkylene, each optionally substituted with one or more substituents selected from halo, C₁₋₆alkyl, C₁₋₆haloalkyl, C₁₋₆alkoxy, hydroxy, aryl, heteroaryl, and C₁₋₆haloalkoxy;
L″ is optionally present and is selected from —O— and —N(C₁₋₆alkyl)-; and
r is an integer from 1 to 20, s is an integer from 0 to 4, and t is an integer from 1 to 4.

Clause 25. The linker of clause 24, wherein A comprises no more than two nitrogen atoms.

Clause 26. The linker of clause 24 or clause 25, wherein A is selected from formulae (2a) to (2c):

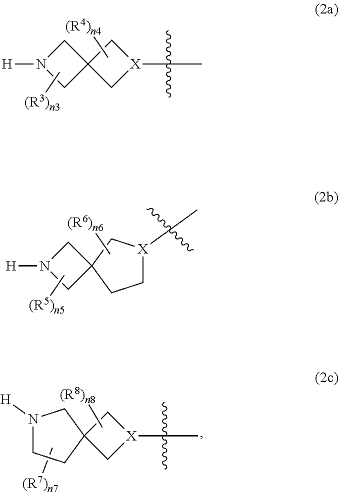

wherein:
   X is N or CH;
each R³ to R⁸ is independently selected from halo, C₁₋₆alkyl, C₁₋₆haloalkyl, C₁₋₆alkoxy, hydroxy, aryl, heteroaryl, and C₁₋₆haloalkoxy;
each n3, n4, n5 and n8 is independently selected from 0 to 2; and each n6 and n7 is independently selected from 0 to 3.

Clause 27. The linker of any one of clauses 24 to 26, wherein the nucleofuge is selected from sulfonate and halo.

Clause 28. The linker of any one of clauses 24 to 27, wherein X¹, L″ and X² are absent.

Clause 29. The linker of any one of clauses 24 to 28, wherein each L′ is independently selected from the group consisting of O(CH₂)ₜ, CH₂, phenylene and pyridinene, each optionally substituted with one or more substituents selected from halo, C₁₋₆alkyl, C₁₋₆haloalkyl, C₁₋₆alkoxy, hydroxy, aryl, heteroaryl, and C₁₋₆haloalkoxy.

Clause 30. The linker of any one of clauses 24 to 29, wherein:

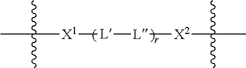

is selected from formulae (3a) to (3f):

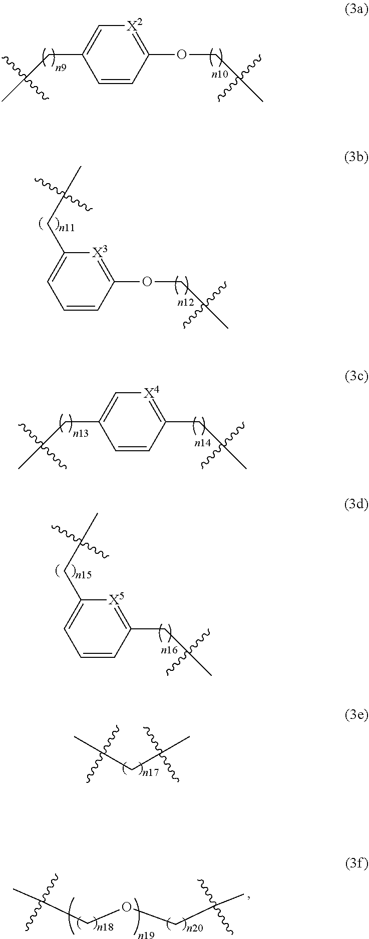

wherein:
each X² to X⁵ is independently selected from N and CH;
each n9 to n16, n18 and n20 is independently selected from 0 to 10; and each n17 and n19 is independently selected from 1 to 10.

Clause 31. The linker of any one of clauses 24 to 30, wherein the linker is of formula (4a) or (4b):

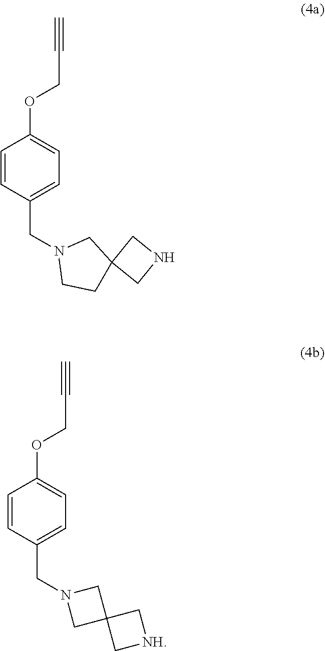 [SMILES: C#CCOc1ccc(CN2CCC3(CNC3)C2)cc1 ; C#CCOc1ccc(CN2CC3(CNC3)C2)cc1]

Clause 32. A method of manufacturing a bifunctional compound, optionally as a one-pot method, the method comprising:
(i) contacting:
(a) a linker of any one of clauses 24 to 31; and
(b) two molecules, one of which comprises a moiety for reacting with one of A and B, and the other of which comprises a moiety for reacting with the other of A and B;
and optionally:
(ii) analysing the resultant mixture for formation of a bifunctional compound comprising each of the two molecules linked together by the linker.

Clause 33. A method of targeted protein degradation or stabilisation, the method comprising:
(i) contacting:
   (a) a linker of any one of clauses 24 to 31; and
   (b) two molecules, one of which comprises a moiety for reacting with one of A and B, and the other of which comprises a moiety for reacting with the other of A and B, and wherein one of the molecules comprises a target protein binder and the other comprises an E3 ubiquitin ligase or a deubiquitinase binder;
(ii) analysing the resultant mixture for formation of a bifunctional compound comprising each of the two molecules linked together by the linker; and
(iii) contacting the bifunctional compound with the target protein.

Clause 34. A kit comprising:
(i) a linker as defined in any one of clauses 24 to 31; and
(ii) a molecule comprising a moiety for reacting with A and/or a molecule comprising a moiety for reacting with B.

### Examples

### Experimental Information

### Abbreviations

DMF Dimethylformamide
DBU 1,8-Diazabicyclo[5.4.0]undec-7-ene
POI Protein of interest
THPTA Tris(3-hydroxypropyltriazolylmethyl)amine
DMSO Dimethylsulfoxide
STAB Sodium triacetoxyborohydride
EtOAc Ethyl acetate
ACN Acetonitrile
DCM Dichloromethane (methylene chloride)
MeOH Methanol
NMR Nuclear magnetic resonance
LCMS Liquid chromatography-mass spectrometry
ESI-MS Electrospray ionisation-mass spectrometry
AcOH Acetic acid
Hp Heptanes
RT Room temperature
SCX Strong-cation exchange
DIPEA N,N-Di-iso-propylethylamine
TFA 2,2,2-Trifluoroacetic acid

High throughput Screening for GSK3 β-HiBiTL: Test compounds were transferred into Echo plate (Beckman, 001-12782, Echo LDV384) using BRAVO automated liquid handling platform which serves as source plate (10 mM stocks in DMSO). Serial dilutions of the compounds were done using Echo550 liquid handler as 30 μM, 10 μM, 3 μM, 1 μM, 0.3 μM, 0.1 μM, 0.03 μM and 0.01 μM in final concentrations in assay plates. One column of each assay plate was reserved for DMSO control treatments. After compounds were serially diluted in assay plates, HEK293 cells (CRL-1573, ATCC) genetically engineered using CRISPR/Cas9 technology to endogenously tag GSK3p protein with HiBiT tag at the C-terminus (Schwinn et al., 2018, CRISPR-mediated tagging of endogenous proteins with a luminescent peptide, ACS Chem. Biol., 13(2), 467-74) were seeded at a density of 5000 cells/well in 50 μL complete media in 384-well TC-grade white plates (Perkin Elmer, Culturplate, 6007619) with the help of Multidrop Combi liquid dispenser and cells were incubated for 24 h with compounds at 37° C. in humidified incubators with 5% CO₂. At the end of the treatment, equal volume of Nano-Glo HiBiT Lytic reagent (Promega N3030) supplied with Nano-Glo HiBiT Lytic substrate and LgBiT Protein were added into each well according to manufacturer's instructions, and cells were incubated on an orbital shaker for 15 seconds and for a further 15 minutes at room temperature. Luminescence signals were measured in an appropriate plate reader (PHERAstar FSX, BMG Labtech) (Dixon et al., 2016, NanoLuc complementation reporter optimized for accurate measurement of protein interactions in cells, ACS Chem. Biol., 11, 400-8). Changes in protein abundance upon compound treatment were further analysed by normalising raw luminescence values to DMSO values and fold changes were plotted using for parameter logistic regression with GraphPad Prism (10.1.2).

General experimental information: All ¹H and ¹³C NMR spectra were recorded at ambient temperature on either Bruker Ascend 500 MHz spectrometers. Chemical shifts (6/ppm) were referenced to the residual solvent peak in ¹H (7.26 ppm for CDCl₃, 2.05 ppm for DMSO-d₆). Coupling constants (J are given in Hz. Signal splitting patterns are described as singlet (s), doublet (d), triplet (t), multiplet (m), broad (br) or a combination thereof.

Liquid chromatography-mass spectrometry (LC-MS) was carried out on a Shimadzu HPLC/MS 2020 equipped with a Hypersil Gold column (1.9 μm particle size, 50×2.1 mm), photodiode array detector and ESI detector. Samples were eluted with either a 3 min or 5 min gradient of 5-95% acetonitrile/water containing 0.1% formic acid at a flow rate of 0.7 mL/min.

Purification: Thin layer chromatography (TLC) was performed on pre-coated alumina plates (Silica gel 60 F254, Merck) and visualized via UV light (UV 254 and/or 365 nm) and/or basic potassium permanganate solution. Flash column chromatography was performed using a Teledyne Isco Combiflash Rf with prepacked Redisep RF Normal phase disposable columns (230-400 mesh, 40-63 mm: SiliCycle). Preparative reverse-phase HLPC purification was performed on Gilson Preparative HPLC System equipped with a Waters XBridge C18 column (100 mm×19 mm; 5 μm particle size) using a gradient from 5 to 95% of acetonitrile in water containing 0.1% formic over 10 min at a flow rate of 25 mL/min unless stated otherwise.

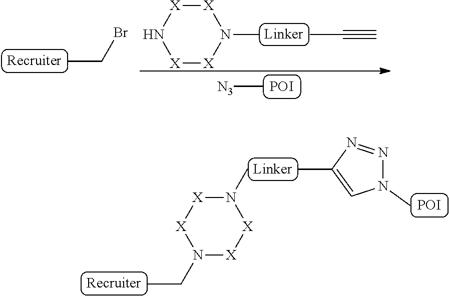

Method A: All linkers were pre-dissolved in DMF with 3.2 equivalents of triethylamine. Reagents were prepared as the following DMF stock solutions unless stated otherwise in 1.9 mL Tri-coded FluidX tubes, 48-format: Recruiter-ligands (0.08 M), linker reagents (0.095 M), POI-Azide (0.1 M), CuSO₄ with THPTA in water (0.05 M CuSO₄, 0.1 M THPTA), sodium ascorbate in water (0.4 M). All liquid transfers were done using either a single channel pipette or multichannel pipette. These were prepared in order to achieve a final Step 1 reaction concentration of 0.01 M and Step 2 reaction concentration of 0.02 M of limiting reagent (amine-alkyne-linker). 18.8 μL of each Recruiter-ligand stock solution was added to a 96-well plate (Para-dox standard 96-position, Parallel synthesis aluminium reaction block, 1 mL, 8×30 mm glass inserts) with Recruiter-ligand A in row A, Recruiter-ligand B in row B, Recruiter-ligand C in row C, and so on. Then, 10.5 μL of each linker stock solution was added to the 96-well plate containing the Recruiter-ligands with linker 1 in column 1, linker 2 in column 2, linker 3 in column 3, and so on. The reaction block was then sealed and left for 24 h at RT. The reaction block was then unsealed and 19.5 μL DMF was added to each well followed by 10 μL of POI-Azide, 10 μL of CuSO₄/THPTA solution, and 7.5 μL of sodium ascorbate, in that order. The reaction plate was then sealed and left for 24 h at RT. Then the reaction plate was unsealed and 1 μL of formic acid was added to each reaction well. To an acidified 96-well SCX plate was added each reaction to a single well and then each well washed twice with 1 mL of ACN before elution with 1 mL of 2 M NH₄OH in acetonitrile. The eluant was then collected into a 2 mL ISOLUTE collection plate and the solvent was evaporated. Following the evaporation of basified ACN, 100 μL of DMSO was added to each well giving a maximum 10 mM DMSO stock solution of each library compound which could be used in desired assays without further manipulation.

Method B: All linkers were pre-dissolved in DMF with 3.2 equivalents of triethylamine. Reagents were prepared as the following DMF stock solutions unless stated otherwise in 1.9 mL Tri-coded FluidX tubes, 48-format: Recruiter-ligands (0.08 M), linker reagents (0.095 M), POI-Azide (0.1 M), CuSO₄ with THPTA in water (0.05 M CuSO₄, 0.1 M THPTA), sodium ascorbate in water (0.4 M) and potassium iodide (0.08 M). All liquid transfers were done using either a single channel pipette or multichannel pipette. These were prepared in order to achieve a final Step 1 reaction concentration of 0.01 M and Step 2 reaction concentration of 0.02 M of limiting reagent (amine-alkyne-linker). 18.8 μL of each Recruiter-ligand stock solution was added to a 96-well plate (Para-dox standard 96-position, Parallel synthesis aluminium reaction block, 1 mL, 8×30 mm glass inserts) with Recruiter-ligand A in row A, Recruiter-ligand B in row B, Recruiter-ligand C in row C, and so on. Then, 10.5 μL of each linker stock solution was added to the 96-well plate containing the Recruiter-ligands with linker 1 in column 1, linker 2 in column 2, linker 3 in column 3, and so on, followed by addition of 1.5 μL of potassium iodide solution to each reaction well. The reaction block was then sealed and left for 24 h at RT. The reaction block was then unsealed and 19.5 μL DMF was added to each well followed by 10 μL of POI-Azide, 10 μL of CuSO₄/THPTA solution, and 7.5 μL of sodium ascorbate, in that order. The reaction plate was then sealed and left for 24 h at RT. Then the reaction plate was unsealed and 1 μL of formic acid was added to each reaction well. To an acidified 96-well SCX plate was added each reaction to a single well and then each well washed twice with 1 mL of ACN before elution with 1 mL of 2 M NH₄OH in acetonitrile. The eluant was then collected into a 2 mL ISOLUTE collection plate and the solvent was evaporated. Following the evaporation of basified ACN, 100 μL of DMSO was added to each well giving a maximum 10 mM DMSO stock solution of each library compound which could be used in desired assays without further manipulation.

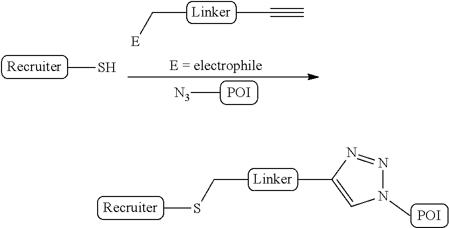

Method C: Reagents were prepared as the following DMF stock solutions unless stated otherwise in 1.9 mL Tri-coded FluidX tubes, 48-format: Thiols (0.33 mM), linkers (0.5 mM), azides (0.53 mM), DBU (50% in DMF), CuSO₄ with THPTA in water (0.22 mM CuSO₄, 0.44 mM THPTA) and sodium ascorbate in water (0.016 mM). All liquid transfers were done using either a single channel pipette or multichannel pipette. 0.008 mmol of linker stock solution was added to each reaction well followed by addition of 0.008 mmol of thiol stock solution and 0.012 mmol of base stock solution to corresponding wells. After 30 minutes of reaction, 0.008 mmol of azide stock solution was added to each reaction well followed by 0.004 mmol CuSO₄ stock solution then 0.016 mmol sodium ascorbate stock solution. The reaction was then left for 1.5 h. The contents of each reaction well was then filtered through silica and dried. DMSO was then added to each product to give a maximum 10 mM DMSO stock solution which could be used in desired assays without further manipulation.

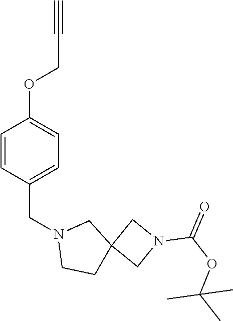 [SMILES: C#CCOc1ccc(CN2CCC3(C2)CN(C(=O)OC(C)(C)C)C3)cc1]

Compound 1: Tert-butyl 7-[(4-prop-2-ynoxyphenyl)methyl]-2,7-diazaspiro[3.4]octane-2-carboxylate: To a solution of 4-prop-2-ynoxybenzaldehyde (1.00 equiv., 1000 mg, 6.24 mmol) in DCM (12.5 mL) was added tert-butyl 6-azaspiro[3.4]octane-2-carboxylate (1.10 equiv., 1451 mg, 6.87 mmol) and acetic acid (1.10 equiv., 0.39 mL, 6.87 mmol) at 0° C. The reaction was then stirred at 0° C. for 1 h. To the solution was then added STAB (3.00 equiv., 3970 mg, 18.7 mmol) in several portions over 30 minutes. The reaction was then stirred at ambient temperature overnight. The reaction was then quenched with saturated NaHCO₃ solution and extracted with DCM. The organic layer was then collected and dried with MgSO₄ before concentrating under reduced pressure. The crude was then purified by basified silica flash column chromatography eluting 40% EtOAc in Hp. The desired fractions were concentrated under reduced pressure to give tert-butyl 7-[(4-prop-2-ynoxyphenyl)methyl]-2,7-diazaspiro[3.4]octane-2-carboxylate (798 mg, 2.24 mmol, 36% yield) as a colourless oil.

¹H NMR (500 MHz, CDCl₃): δ 7.22 (d, J=8.5 Hz, 2H), 6.92 (d, J=8.6 Hz, 2H), 4.68 (d, J=2.4 Hz, 2H), 3.81 (s, 4H), 3.52 (s, 2H), 2.65 (s, 2H), 2.56 (t, J=7.1 Hz, 2H), 2.52 (t, J=2.4 Hz, 1H), 2.03 (t, J=7.1 Hz, 2H), 1.42 (s, 9H).

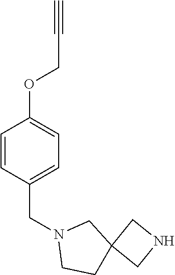 [SMILES: C#CCOc1ccc(CN2CCC3(CNC3)C2)cc1]

Compound 2: 6-[(4-Prop-2-ynoxyphenyl)methyl]-2,6-diazaspiro[3.4]octane: To a solution of tert-butyl 7-[(4-prop-2-ynoxyphenyl)methyl]-2,7-diazaspiro[3.4]octane-2-carboxylate (1.00 equiv., 578 mg, 1.62 mmol) in DCM (2.03 mL) was added 2,2,2-trifluoroacetic acid (7.50 equiv., 0.93 mL, 12.2 mmol), The reaction was then stirred at ambient temperature overnight. The reaction was concentrated under reduced pressure and the residue was taken up in 4 mL of ACN/water (1:1) and subjected to reverse-phase flash column chromatography (formic acid 0.1% in water/ACN). The desired fractions were concentrated to give 6-[(4-prop-2-ynoxyphenyl)methyl]-2,6-diazaspiro[3.4]octane (213 mg, 0.831 mmol, 51% yield) as a white solid.

¹H NMR (500 MHz, CDCl₃): δ 7.22 (d, J=8.5 Hz, 2H), 6.91 (d, J=8.2 Hz, 2H), 4.67 (d, J=2.4 Hz, 2H), 3.60 (m, 4H), 3.52 (s, 2H), 2.72 (s, 2H), 2.54-2.48 (m, 3H), 2.04 (t, J=7.0 Hz, 2H).

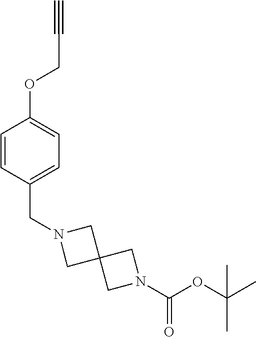 [SMILES: C#CCOc1ccc(CN2CC3(C2)CN(C(=O)OC(C)(C)C)C3)cc1]

Compound 3: Tert-butyl 6-[(4-prop-2-ynoxyphenyl)methyl]-2,6-diazaspiro[3.3]heptane-2-carboxylate: To a solution of 4-prop-2-ynoxybenzaldehyde (1.00 equiv., 1000 mg, 6.24 mmol) in DCM (12.5 mL) was added tert-butyl 2,6-diazaspiro[3.3]heptane-2-carboxylate; oxalic acid (1.10 equiv., 1980 mg, 6.87 mmol) and acetic acid (1.10 equiv., 0.39 mL, 6.87 mmol) at 0° C. The reaction was then stirred at 0° C. for 1 h. To the solution was then added STAB (3.00 equiv., 3970 mg, 18.7 mmol) in several portions over 30 minutes. The reaction was then stirred at ambient temperature overnight. The reaction was then quenched with saturated NaHCO₃ solution and extracted with DCM. The organic layer was then collected and dried with MgSO₄ before concentrating under reduced pressure. The crude was then purified by amine flash column chromatography eluting 0-100% EtOAc in Hp. The desired fractions were concentrated under reduced pressure to give tert-butyl 6-[(4-prop-2-ynoxyphenyl)methyl]-2,6-diazaspiro[3.3]heptane-2-carboxylate (776 mg, 2.27 mmol, 36% yield) as a colourless oil.

¹H NMR (500 MHz, CDCl₃): δ 7.17 (d, J=8.3 Hz, 2H), 6.92 (d, J=8.7 Hz, 2H), 4.67 (d, J=2.4 Hz, 2H), 3.97 (s, 4H), 3.48 (s, 2H), 3.29 (s, 4H), 2.51 (t, J=2.4 Hz, 1H), 1.42 (s, 9H).

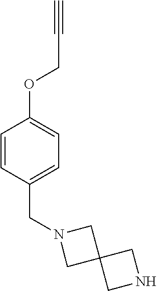 [SMILES: C#CCOc1ccc(CN2CC3(CNC3)C2)cc1]

Compound 4: 2-[(4-Prop-2-ynoxyphenyl)methyl]-2,6-diazaspiro[3.3]heptane: To a solution of tert-butyl 6-[(4-prop-2-ynoxyphenyl)methyl]-2,6-diazaspiro[3.3]heptane-2-carboxylate (1.00 equiv., 519 mg, 1.5 mmol) in DCM (1.9 mL) was added 2,2,2-trifluoroacetic acid (7.50 equiv., 0.87 mL, 11.4 mmol). The reaction was then stirred at ambient temperature overnight. The reaction was concentrated under reduced pressure and the residue was taken up in 4 mL of ACN/water (1:1) mixture and subjected to reverse-phase flash column chromatography (formic acid 0.1% in water/ACN). The desired fractions were concentrated under reduced pressure to give 2-[(4-prop-2-ynoxyphenyl)methyl]-2,6-diazaspiro[3.3]heptane (231 mg, 0.953 mmol, 63% yield) as a white solid.

¹H NMR (500 MHz, CDCl₃): δ 7.18 (d, J=8.5 Hz, 2H), 6.91 (d, J=8.4 Hz, 2H), 4.67 (d, J=2.4 Hz, 2H), 3.73 (s, 4H), 3.48 (s, 2H), 3.30 (s, 4H), 2.51 (d, J=2.5 Hz, 1H).

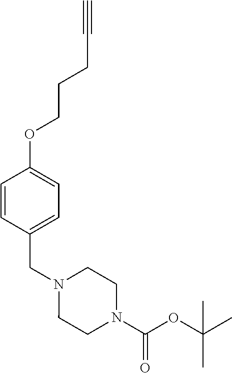 [SMILES: C#CCCCOc1ccc(CN2CCN(C(=O)OC(C)(C)C)CC2)cc1]

Compound 5: Tert-butyl 4-[(4-pent-4-ynoxyphenyl)methyl]piperazine-1-carboxylate: To a solution of 4-pent-4-ynoxybenzaldehyde (1.00 equiv., 940 mg, 4.99 mmol) in DCM (10.0 mL) was added i-butyl piperazine-1-carboxylate (1.10 equiv., 1023 mg, 5.49 mmol) and acetic acid (1.10 equiv., 0.31 mL, 5.49 mmol) at 0° C. The reaction was then stirred at 0° C. for 1 hour. To the solution was then added STAB (3.00 equiv., 3175 mg, 15.0 mmol) in several portions over 30 minutes. The reaction was then stirred at ambient temperature overnight. The reaction was then quenched with saturated NaHCO₃ solution and extracted with DCM. The organic layer was then collected and dried with MgSO₄ before concentrating under reduced pressure. The crude was then purified by amine flash column chromatography eluting 40% EtOAc in Hp. The desired fractions were concentrated under reduced pressure give tert-butyl 4-[(4-pent-4-ynoxyphenyl)methyl]piperazine-1-carboxylate (558 mg, 1.56 mmol, 31% yield) as a colourless oil.

LCMS: 358.48 [M+H⁺]: 359.

¹H NMR (500 MHz, CDCl₃): δ 7.20 (d, J=8.5 Hz, 2H), 6.85 (d, J=8.6 Hz, 2H), 4.05 (t, J=6.1 Hz, 2H), 3.47 (s, 2H), 3.42 (t, J=5.1 Hz, 4H), 2.43-2.37 (m, 6H), 2.03-1.98 (m, 2H), 1.96 (t, J=2.7 Hz, 1H), 1.45 (s, 9H).

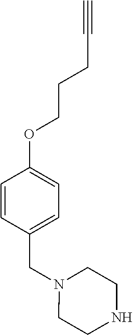 [SMILES: C#CCCCOc1ccc(CN2CCNCC2)cc1]

Compound 6: 1-[(4-Pent-4-ynoxyphenyl)methyl]piperazine: To a solution of tert-butyl 4-[(4-pent-4-ynoxyphenyl)methyl]piperazine-1-carboxylate (1.00 equiv., 558 mg, 1.56 mmol) in DCM (3.9 mL) was added hydrogen chloride (10.0 equiv., 3.9 mL, 15.6 mmol). The reaction was concentrated under reduced pressure and the residue was taken up in 4 mL of ACN/water (1:1) and subjected to reverse-phase flash column chromatography (formic acid 0.1% in water/ACN). The desired fractions were concentrated under reduced pressure to give 1-[(4-pent-4-ynoxyphenyl)methyl]piperazine (340 mg, 1.32 mmol, 85% yield) as a white solid.

LCMS: 259.1 [M+H⁺]: 258.36.

¹H NMR (500 MHz, CDCl₃): δ 7.18 (d, J=8.4 Hz, 2H), 6.85 (d, J=8.4 Hz, 2H), 4.05 (t, J=6.1 Hz, 2H), 3.49 (s, 2H), 3.17 (t, J=5.1 Hz, 4H), 2.71 (t, J=5.1 Hz, 4H), 2.40 (td, J=7.0, 2.7 Hz, 2H), 2.00 (q, J=6.5 Hz, 2H), 1.96 (t, J=2.7 Hz, 1H).

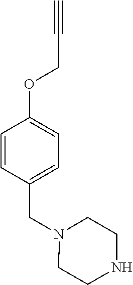 [SMILES: C#CCOc1ccc(CN2CCNCC2)cc1]

Compound 7: 1-[(4-Prop-2-ynoxyphenyl)methyl]piperazine: To a solution of 4-prop-2-ynoxybenzaldehyde (1.00 equiv., 428 mg, 2.7 mmol) in DCM (5.4 mL) was added tert-butyl piperazine-1-carboxylate (1.10 equiv., 548 mg, 2.94 mmol) and acetic acid (1.10 equiv., 0.17 mL, 2.9 mmol) at 0° C. The reaction was then stirred at 0° C. for 1 h. To the solution was then added STAB (3.00 equiv., 1701 mg, 8.02 mmol) in several portions over 30 minutes. The reaction was then stirred at ambient temperature overnight. The reaction was then quenched with saturated NaHCO₃ solution and extracted with DCM. The organic layer was then collected and dried with MgSO₄ before concentrating under reduced pressure. The crude was then purified by amine flash column chromatography eluting 40% EtOAc in Hp. The desired fractions were concentrated under reduced pressure and taken forward to the next step without further purification. The crude tert-butyl 4-[(4-prop-2-ynoxyphenyl)methyl]piperazine-1-carboxylate (1.00 equiv., 884 mg, 2.67 mmol) was taken up in DCM (3.4 mL) and 2,2,2-trifluoroacetic acid (7.50 equiv., 1.5 mL, 20.1 mmol) was added. The reaction was then stirred at ambient temperature overnight. The reaction was then concentrated under reduced pressure and the residue was taken up in 4 mL of ACN/water (1:1) mixture and subjected to a reverse-phase flash column chromatography (formic acid 0.1% in water/ACN). The desired fractions were concentrated under reduced pressure give 1-[(4-prop-2-ynoxyphenyl)methyl]piperazine (430 mg, 1.87 mmol, 69% yield) as a white solid.

LCMS: 230.31 [M+H⁺]: 231.

¹H NMR (500 MHz, CDCl₃): δ 7.24 (d, J=8.7 Hz, 2H), 6.92 (d, J=8.6 Hz, 2H), 4.68 (d, J=2.4 Hz, 2H), 3.43 (s, 2H), 2.88 (t, J=4.9 Hz, 4H), 2.51 (t, J=2.4 Hz, 1H), 2.46-2.33 (br. m, 4H).

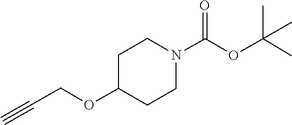 [SMILES: C#CCOC1CCN(C(=O)OC(C)(C)C)CC1]

Compound 8: Tert-butyl 4-prop-2-ynoxypiperidine-1-carboxylate: To a solution of tert-butyl 4-hydroxypiperidine-1-carboxylate (1.00 equiv., 500 mg, 2.48 mmol) in DMF (1 mL) was added sodium hydride (1.10 equiv., 109 mg, 2.73 mmol) at 0° C. and stirred for 10 minutes. Then 3-bromoprop-1-yne (0.800 equiv., 0.18 mL, 1.99 mmol) was added dropwise to the solution at 0° C. and the reaction was then stirred at ambient temperature overnight. The reaction was then quenched with a mixture of saturated aqueous NH₄Cl solution and diluted with Et₂O, then the organic layer was separated. The organic layer was then washed once with water then once with brine. The organics were then dried with MgSO₄ before concentrating under reduced pressure. The crude was then purified by flash column chromatography eluting 40% EtOAc in Hp. The desired fractions were concentrated under reduced pressure give tert-butyl 4-prop-2-ynoxypiperidine-1-carboxylate (400 mg, 1.67 mmol, 67% yield) as a yellow oil. The compound was then taken into the next step without further purification.

¹H NMR (500 MHz, CDCl₃): δ 4.19 (d, J=2.4 Hz, 2H), 3.84-3.73 (m, 2H), 3.73-3.64 (m, 2H), 3.15-3.05 (m, 2H), 2.41 (t, J=2.4 Hz, 1H), 1.90-1.80 (m, 3H), 1.57-1.51 (m, 2H), 1.45 (s, 10H).

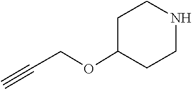

Compound 9: 4-Prop-2-ynoxypiperidin-1-ium: To a solution of tert-butyl 4-prop-2-ynoxypiperidine-1-carboxylate (1.00 equiv., 400 mg, 1.67 mmol) in DCM (8.4 mL) was added hydrogen chloride (10.0 equiv., 4.2 mL, 16.7 mmol) at 0° C. The reaction was then stirred overnight. The reaction was then concentrated under reduced pressure to give 4-prop-2-ynoxypiperidin-1-ium (220 mg, 1.57 mmol, 94% yield).

¹H NMR (500 MHz, CDCl₃): δ 4.17 (d, J=2.4 Hz, 2H), 3.90 (m, 1H), 3.35-3.24 (m, 2H), 3.24-3.14 (m, 2H), 2.43 (t, J=2.4 Hz, 1H), 2.20-2.10 (m, 2H), 2.07-1.98 (m, 2H).

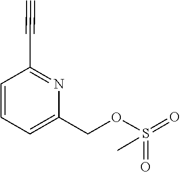

Compound 10: (6-Ethynyl-2-pyridyl)methyl methanesulfonate: To a solution of (6-ethynyl-2-pyridyl)methanol (1.00 equiv., 450 mg, 3.38 mmol) in DCM (7.5 mL) was added methanesulfonyl chloride (3.00 equiv., 0.78 mL, 10.1 mmol) at −78° C. under a nitrogen atmosphere. The reaction was then stirred for 1 h at −78° C. The reaction was then quenched with 5 mL of saturated NaHCO₃ solution and extracted with 10 mL of DCM. The organics were then separated and dried with MgSO₄ before concentrating under reduced pressure. The crude was then purified by flash column chromatography (EtOAc in Hp). The desired fractions were concentrated under reduced pressure give (6-ethynyl-2-pyridyl)methyl methanesulfonate (231 mg, 1.09 mmol, 32% yield) as a dark red oil.

LCMS: 211.24 [M+H⁺]: 211.9.

¹H NMR (500 MHz, CDCl₃): δ 7.75 (t, J=7.8 Hz, 1H), 7.48 (d, J=7.8 Hz, 2H), 5.32 (s, 2H), 3.20 (s, 1H), 3.11 (s, 3H).

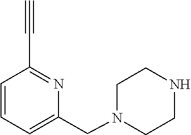

Compound 11: 1-[(6-Ethynyl-2-pyridyl)methyl]piperazine: To a solution of (6-ethynyl-2-pyridyl)methyl methanesulfonate (1.00 equiv., 231 mg, 1.09 mmol) in DMF (2.2 mL) was added potassium iodide (0.200 equiv., 36 mg, 0.219 mmol) and tert-butyl piperazine-1-carboxylate (2.00 equiv., 407 mg, 2.19 mmol). The reaction was then stirred overnight at 80° C. The reaction was taken up in EtOAc (50 mL) and the organics washed with 2×40 mL water then 1×40 mL saturated brine solution. The organics were then separated and dried with MgSO₄ before concentrating under reduced pressure to give tert-butyl 4-[(6-ethynyl-2-pyridyl)methyl]piperazine-1-carboxylate (190 mg) which was taken into the next step without further purification. The crude was then dissolved in DCM (3.15 mL) and hydrogen chloride (30.0 equiv., 4.7 mL, 18.9 mmol) was added. The reaction was then stirred for 3 h. Thereafter, the reaction was concentrated under reduced pressure and subjected to an ion-exchange column. The compound was eluted with 3.5 N NH₃ in methanol. The eluant was then concentrated under reduced pressure to give 1-[(6-ethynyl-2-pyridyl)methyl]piperazine (121 mg, 0.601 mmol, 95% yield) as a white solid.

¹H NMR (500 MHz, CDCl₃): δ 7.63 (t, J=7.7 Hz, 1H), 7.48 (d, J=7.8 Hz, 1H), 7.36 (d, J=7.6 Hz, 1H), 3.66 (s, 2H), 3.13 (s, 1H), 2.90 (t, J=4.9 Hz, 4H), 2.48 (t, J=4.7 Hz, 4H).

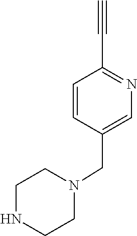

Compound 12: 1-((6-Ethynylpyridin-3-yl)methyl)piperazine: Synthesised according to the procedure described in CN113683611 A.

LCMS: 201.27 [M+H⁺]: 202.

¹H NMR (500 MHz, CDCl₃): δ 8.67 (d, J=2.3 Hz, 1H), 7.74 (dd, J=8.0, 2.2 Hz, 1H), 7.40 (d, J=7.9 Hz, 1H), 3.65 (s, 2H), 3.19 (s, 1H), 2.93 (t, J=4.9 Hz, 4H), 2.49 (s, 4H).

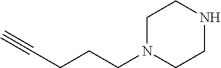

Compound 13: 1-(Pent-4-yn-1-yl)piperazine: Synthesised according to the procedure described in WO2006105372 A2.

¹H NMR (500 MHz, CDCl₃): 2.87 (t, J=4.9 Hz, 4H), 2.46-2.35 (m, 6H), 2.22 (td, J=7.2, 2.6 Hz, 2H), 1.93 (t, J=2.7 Hz, 1H), 1.72-1.67 (m, 2H).

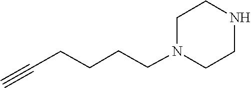

Compound 14: 1-Hex-5-ynylpiperazine: Synthesised according to the procedure described in Nat. Chem., 2024, 16, 183.

LCMS: 166 [M+H⁺]: 167.

¹H NMR (500 MHz, CDCl₃): δ 2.89 (t, J=4.9 Hz, 4H), 2.51-2.36 (br. m, 4H), 2.32 (t, J=7.2 Hz, 2H), 2.21 (td, J=6.9, 2.7 Hz, 2H), 1.94 (t, J=2.6 Hz, 1H), 1.65-1.58 (m, 2H), 1.56-1.53 (m, 2H).

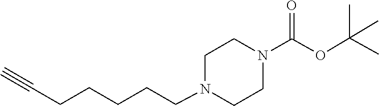 [SMILES: C#CCCCCCN1CCN(C(=O)OC(C)(C)C)CC1]

Compound 15: Tert-butyl 4-hept-6-ynylpiperazine-1-carboxylate: To a solution of hept-6-ynyl methanesulfonate (1.00 equiv., 953 mg, 5.01 mmol) in ACN (20 mL) was added tert-butyl piperazine-1-carboxylate (1.20 equiv., 1119 mg, 6.01 mmol) and triethylamine (2.00 equiv., 1.4 mL, 10.0 mmol). The reaction was then stirred at 90° C. overnight. Thereafter, the reaction was concentrated under reduced and then purified by flash column chromatography eluting 50% EtOAc in Hp. The desired fractions were concentrated under reduced pressure give tert-butyl 4-hept-6-ynylpiperazine-1-carboxylate (800 mg, 2.85 mmol, 57% yield) as a yellow oil.

¹H NMR (500 MHz, CDCl₃): δ 3.43 (t, J=5.1 Hz, 4H), 2.43-2.30 (m, 6H), 2.18 (td, J=7.1, 2.7 Hz, 2H), 1.93 (t, J=2.6 Hz, 1H), 1.58-1.48 (m, 4H), 1.45 (s, 9H), 1.44-1.38 (m, 2H).

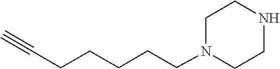

Compound 16: 1-Hept-6-ynylpiperazine: To a solution of tert-butyl 4-hept-6-ynylpiperazine-1-carboxylate (1.00 equiv., 800 mg, 2.85 mmol) in DCM (3.6 mL) was added hydrogen chloride (10.0 equiv., 7.1 mL, 28.5 mmol) at 0° C. and stirred overnight.

The reaction was then concentrated under reduced pressure to give 1-hept-6-ynylpiperazin-4-ium chloride (618 mg, 2.85 mmol, quantitative yield) as a white solid.

LCMS: 180.16 [M+H⁺]: 181.

¹H NMR (500 MHz, CDCl₃): δ 2.89 (t, J=4.9 Hz, 4H), 2.47-2.34 (br. m, 4H), 2.33-2.28 (m, 2H), 2.18 (td, J=7.1, 2.7 Hz, 2H), 1.93 (t, J=2.6 Hz, 1H), 1.56-1.48 (m, 4H), 1.45-1.37 (m, 2H).

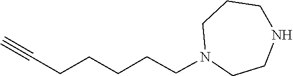

Compound 17: 1-Hept-6-ynyl-1,4-diazepane: To a solution of hept-6-ynyl methanesulfonate (1.00 equiv., 1000 mg, 5.26 mmol) in dry ACN (21.1 mL) was added tert-butyl 1,4-diazepane-1-carboxylate (1.20 equiv., 1.2 mL, 6.31 mmol) and triethylamine (2.00 equiv., 1.5 mL, 10.5 mmol). The reaction was then stirred at 90° C. overnight. The reaction was then concentrated under reduced pressure and the crude material was purified by flash column chromatography eluting at 10% MeOH in DCM. The desired fractions were concentrated under reduced pressure to give crude tert-butyl 4-hept-6-ynyl-1,4-diazepane-1-carboxylate (500 mg). To the crude material in DCM (4 mL) was added hydrogen chloride (10.0 equiv., 4.2 mL, 17.0 mmol). The reaction was then stirred overnight at ambient temperature. The reaction was then concentrated under reduced pressure and purified by SCX-chromatography eluting with 2 M NH₄ in methanol. The eluant was then concentrated under reduced pressure to give 1-hept-6-ynyl-1,4-diazepane (321 mg, 1.65 mmol, 97% yield) as a white solid.

¹H NMR (500 MHz, CDCl₃): δ 2.95-2.88 (m, 4H), 2.70-2.62 (m, 4H), 2.51-2.45 (m, 2H), 2.19 (td, J=7.1, 2.7 Hz, 2H), 1.93 (t, J=2.7 Hz, 1H), 1.76 (p, J=6.0 Hz, 2H), 1.58-1.51 (m, 2H), 1.51-1.45 (m, 2H), 1.44-1.35 (m, 2H).

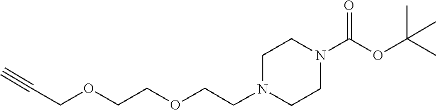 [SMILES: C#CCOCCOCCN1CCN(C(=O)OC(C)(C)C)CC1]

Compound 18: Tert-butyl 4-[2-(2-prop-2-ynoxyethoxy)ethyl]piperazine-1-carboxylate: Synthesised according to the procedure described in WO2021067606 A1.

¹H NMR (500 MHz, CDCl₃): δ 4.17 (d, J=2.5 Hz, 2H), 3.68-3.64 (m, 2H), 3.64-3.57 (m, 4H), 3.39 (t, J=6.8 Hz, 5H), 2.58 (t, J=5.8 Hz, 2H), 2.44-2.39 (m, 4H), 1.43 (s, 9H).

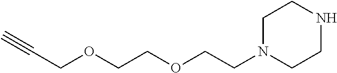

Compound 19: 1-[2-(2-Prop-2-ynoxyethoxy)ethyl]piperazin-4-ium,2,2,2-trifluoroacetate: To a solution of tert-butyl 4-[2-(2-prop-2-ynoxyethoxy)ethyl]piperazine-1-carboxylate (1.00 equiv., 500 mg, 1.60 mmol) in DCM (2.0 mL) was added 2,2,2-trifluoroacetic acid (15.0 equiv., 1.8 mL, 24.0 mmol). The reaction was then stirred at ambient temperature overnight. The reaction was then concentrated under reduced pressure. The crude material was then purified by reverse phase flash column chromatography eluting in ACN/water (0.1% NH₄OH in water/ACN). The desired fractions were concentrated under reduced pressure give 1-[2-(2-prop-2-ynoxyethoxy)ethyl]piperazine (330 mg, 1.55 mmol, 97% yield) as a colourless oil.

¹H NMR (500 MHz, CDCl₃): δ 4.17 (d, J=2.4 Hz, 2H), 3.67-3.65 (m, 2H), 3.62-3.58 (m, 4H), 3.43-3.40 (m, 1H), 2.86 (t, J=4.9 Hz, 4H), 2.55 (t, J=6.0 Hz, 2H), 2.49-2.41 (m, 4H).

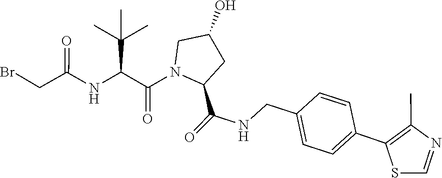 [SMILES: Cc1ncsc1-c1ccc(CNC(=O)[C@@H]2C[C@@H](O)CN2C(=O)[C@@H](NC(=O)CBr)C(C)(C)C)cc1]

Compound 20: (2S,4R)-1-[(2S)-2-[(2-Bromoacetyl)amino]-3,3-dimethyl-butanoyl]-4-hydroxy-N-[[4-(4-methylthiazol-5-yl)phenyl]methyl]pyrrolidine-2-carboxamide: Synthesised according to the procedure described in WO2022125804 A1.

LCMS: 551.5 [M+H⁺]: 552.

¹H NMR (500 MHz, CDCl₃): δ 8.69 (s, 1H), 7.39-7.33 (m, 4H), 7.23 (t, J=6.0 Hz, 1H), 7.09 (d, J=8.6 Hz, 1H), 4.73 (t, J=7.8 Hz, 1H), 4.61-4.52 (m, 2H), 4.47 (d, J=8.7 Hz, 1H), 4.38-4.31 (m, 1H), 4.03-3.99 (m, 1H), 3.88 (s, 2H), 3.68-3.62 (m, 1H), 2.62-2.55 (m, 1H), 2.52 (s, 3H), 2.17-2.09 (m, 1H), 0.95 (s, 9H).

Overall synthetic route for compound 26, 1-[6-(bromomethyl)-1-methyl-indazol-3-yl]hexahydropyrimidine-2,4-dione:

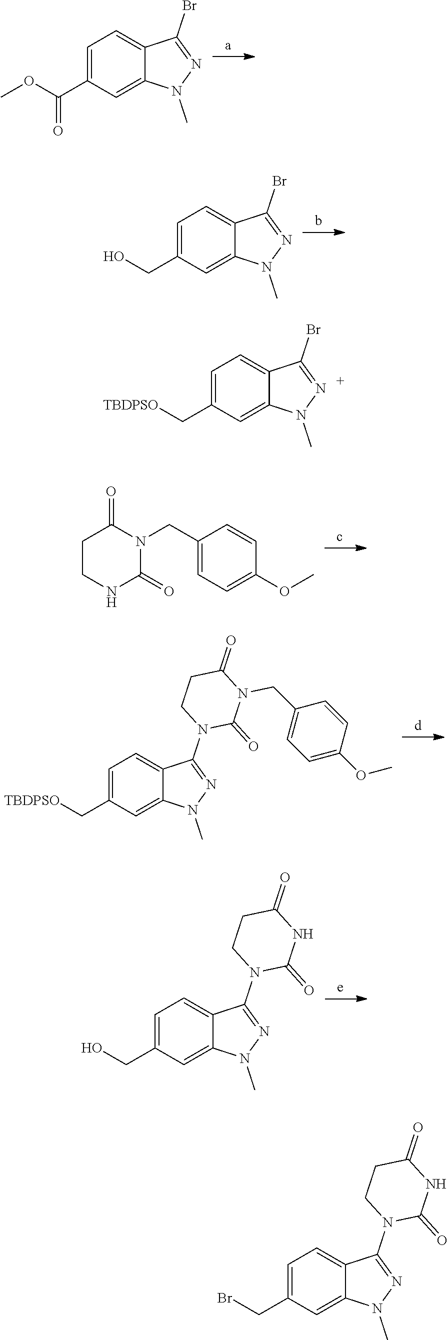

(a) LiAlH₄, THF, −78° C.; (b) Tert-butyl-diphenyl-silyl chloride (TBDPSCl), imidazole, DCM, 0° C.; (c) Cs₂CO₃, CuI, trans-N,N-dimethylcyclohexane-1,2-diamine, 1,4-dioxane, 80° C.; (d) Triflic acid, TFA, 70° C.; and (e) HBr in acetic acid, 90° C.

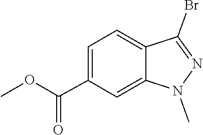

Compound 21: Methyl 3-bromo-1-methyl-indazole-6-carboxylate: Synthesised according to the procedure described in CN115260180 A.

LCMS: 269.1 [M+H⁺]: 271.

¹H NMR (500 MHz, CDCl₃): δ 8.16 (t, J=1.1 Hz, 1H), 7.87 (dd, J=8.5, 1.2 Hz, 1H), 7.66 (d, J=0.8 Hz, 1H), 4.12 (s, 3H), 3.99 (s, 3H).

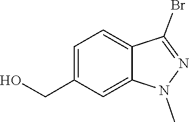

Compound 22: (3-Bromo-1-methyl-indazol-6-yl)methanol: To a solution of methyl 3-bromo-1-methyl-indazole-6-carboxylate (1.00 equiv., 4450 mg, 16.5 mmol) in THE (82.7 mL) at −78° C. was added LiAlH₄ (2.00 equiv., 33 mL, 33.1 mmol) slowly. The resulting mixture was stirred for 30 min at −78° C. After stirring was ended, the reaction mixture was quenched using sodium sulfate decahydrate at −78° C. and then filtered through Celite. The filtrate was then concentrated under reduced pressure. The crude material was then purified by flash column chromatography eluting 60% EtOAc in Hp. The desired fractions were concentrated under reduced pressure give (3-bromo-1-methyl-indazol-6-yl)methanol (2460 mg, 10.2 mmol, 62% yield) as a white solid.

LCMS: 239.99 [M+H⁺]: 241.

¹H NMR (500 MHz, CDCl₃): δ 7.54 (d, J=0.8 Hz, 1H), 7.38 (s, 1H), 7.14 (d, J=1.2 Hz, 1H), 4.86 (d, J=0.9 Hz, 2H), 4.00 (s 3H), 2.22 (t J=5.8 Hz, 1H).

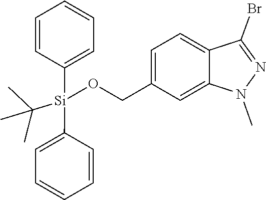 [SMILES: Cn1nc(Br)c2ccc(CO[Si](c3ccccc3)(c3ccccc3)C(C)(C)C)cc21]

Compound 23: (3-Bromo-1-methyl-indazol-6-yl)methoxy-tert-butyl-diphenyl-silane: To a stirred solution of (3-bromo-1-methyl-indazol-6-yl)methanol (1.00 equiv., 2463 mg, 10.2 mmol) in dry DCM (51.1 mL) was added imidazole (2.20 equiv., 1530 mg, 22.5 mmol) and then stirred for 5 minutes at 0° C. To the stirred reaction was added tert-butyl-chloro-diphenyl-silane (1.10 equiv., 2.9 mL, 11.2 mmol) dropwise and stirred at 0° C. for 1 h. The reaction was then concentrated under reduced pressure and taken up in 30 mL of EtOAc and washed once with 20 mL of water and then with 20 mL of brine. The organics were then dried with MgSO₄ and then concentrated under reduced pressure to give (3-bromo-1-methyl-indazol-6-yl)methoxy-tert-butyl-diphenyl-silane (4830 mg, 10.1 mmol, 99% yield).

LCMS: 479.49 [M+H⁺]: 481.

¹H NMR (500 MHz, CDCl₃): δ 7.72-7.68 (m, 4H), 7.53 (d, J=0.7 Hz, 1H), 7.47-7.41 (m, 2H), 7.41-7.36 (m, 5H), 7.09 (d, J=1.2 Hz, 1H), 4.92 (s, 2H), 4.02 (s, 3H), 1.13 (s, 9H).

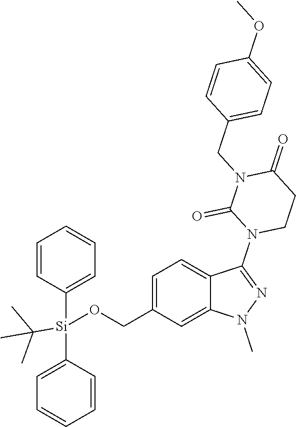 [SMILES: COc1ccc(CN2C(=O)CCN(c3nn(C)c4cc(CO[Si](c5ccccc5)(c5ccccc5)C(C)(C)C)ccc34)C2=O)cc1]

Compound 24: 1-[6-[[Tert-butyl(diphenyl)silyl]oxymethyl]-1-methyl-indazol-3-yl]-3-[(4-methoxyphenyl)methyl]hexahydropyrimidine-2,4-dione: To a microwave vial containing 1,4-dioxane (11.7 mL) was added (3-bromo-1-methyl-indazol-6-yl)methoxy-tert-butyl-diphenyl-silane (1.20 equiv., 1350 mg, 2.82 mmol), 3-[(4-methoxyphenyl)methyl]hexahydropyrimidine-2,4-dione (1.00 equiv., 550 mg, 2.35 mmol), caesium carbonate (2.00 equiv., 1530 mg, 4.70 mmol), copper iodide (0.200 equiv., 89 mg, 0.470 mmol) and rac-N1,N2-dimethylcyclohexane-1,2-diamine (0.200 equiv., 74 μL, 0.470 mmol). The reaction vessel was then evacuated and backfilled with nitrogen three times. The reaction was then stirred at 80° C. under inert atmosphere overnight. Thereafter, the reaction was analysed by LCMS which indicated full consumption of starting material, and the reaction was then filtered through cotton wool and the organics were then concentrated under reduced pressure. The crude material was then purified by flash column chromatography eluting 100% EtOAc in Hp. The desired fractions were concentrated under reduced pressure give 1-[6-[[tert-butyl(diphenyl)silyl]oxymethyl]-1-methyl-indazol-3-yl]-3-[(4-methoxyphenyl)methyl]hexahydropyrimidine-2,4-dione (1202 mg 1.90 mmol, 81% yield) as a colourless oil.

LCMS: 632.84 [M+H⁺]: 633.

¹H NMR (500 MHz, CDCl₃): δ 7.73-7.67 (m, 4H), 7.57 (d, J=0.8 Hz, 1H), 7.47-7.41 (m, 4H), 7.44-7.39 (m, 5H), 7.06 (d, J=1.3 Hz, 1H), 6.86-6.81 (m, 2H), 5.02 (s, 2H), 4.90 (s, 2H), 4.00 (t, J=6.7 Hz, 2H), 3.97 (s, 3H), 3.78 (s, 3H), 2.93 (t, J=6.7 Hz, 2H), 1.12 (s, 9H).

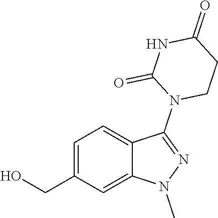 [SMILES: Cn1nc(N2CCC(=O)NC2=O)c2ccc(CO)cc21]

Compound 25: 1-[6-(Hydroxymethyl)-1-methyl-indazol-3-yl]hexahydropyrimidine-2,4-dione: To a solution of 1-[6-[[tert-butyl(diphenyl)silyl]oxymethyl]-1-methyl-indazol-3-yl]-3-[(4-methoxyphenyl)methyl]hexahydropyrimidine-2,4-dione (1.00 equiv., 2401 mg, 3.79 mmol) in 2,2,2-trifluoroacetic acid (54.2 mL) was added trifluoromethanesulfonic acid (15.0 equiv., 5.0 mL, 56.9 mmol) slowly and the reaction was heated to 60° C. for 5 h. Thereafter, the reaction was concentrated under reduced pressure, taken up in 50 mL of EtOAc and concentrated again under reduced pressure. The crude was then purified by flash column chromatography eluting with 15% MeOH in DCM. The desired fractions were concentrated under reduced pressure give crude 1-[6-(hydroxymethyl)-1-methyl-indazol-3-yl]hexahydropyrimidine-2,4-dione. The material was then taken into the next step without further purification.

LCMS: 274.28 [M+H⁺]: 275.1.

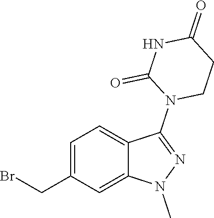 [SMILES: Cn1nc(N2CCC(=O)NC2=O)c2ccc(CBr)cc21]

Compound 26: 1-[6-(Bromomethyl)-1-methyl-indazol-3-yl]hexahydropyrimidine-2,4-dione: To a solution of hydrogen bromide (281 equiv., 17 mL, 666 mmol) was added 1-[6-(hydroxymethyl)-1-methyl-indazol-3-yl]hexahydropyrimidine-2,4-dione (1.00 equiv., 650 mg, 2.37 mmol) and then stirred at 90° C. for 30 minutes. Thereafter, the reaction was monitored by LCMS which indicated full consumption of starting material and formation of product. The reaction was then taken up in distilled water (20 mL) and washed with EtOAc (3×50 mL). The organic layers were then dried with MgSO₄ and concentrated under reduced pressure. The crude was then purified by reverse phase flash column chromatography (formic acid 0.1% in water/ACN). The desired fractions were concentrated under reduced pressure give 1-[6-(bromomethyl)-1-methyl-indazol-3-yl]hexahydropyrimidine-2,4-dione (234 mg, 0.694 mmol, 29% yield) as a white solid.

LCMS: 337.18 [M+H⁺]: 337.

¹H NMR (500 MHz, DMSO-d₆): δ 10.58 (s, 1H), 7.72 (s, 1H), 7.64 (d, J=8.4 Hz, 1H), 7.18 (d, J=1.4 Hz, 1H), 4.85 (s, 2H), 3.98 (s, 3H), 3.92 (t, J=6.7 Hz, 2H), 2.76 (t, J=6.6 Hz, 2H).

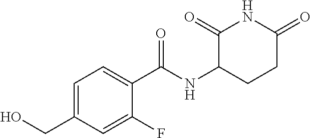 [SMILES: O=C1CCC(NC(=O)c2ccc(CO)cc2F)C(=O)N1]

Compound 27: N-(2,6-Dioxo-3-piperidyl)-2-fluoro-4-(hydroxymethyl)benzamide: To a solution of 2-fluoro-4-(hydroxymethyl)benzoic acid (1.00 equiv., 400 mg, 2.35 mmol) in dry DMF (15.7 mL) was added DIPEA (4.00 equiv., 1.6 mL, 9.40 mmol) dropwise at 0° C. under an inert atmosphere and then stirred for 5 minutes. Thereafter, [dimethylamino(triazolo[4,5-b]pyridin-3-yloxy)methylene]-dimethyl-ammonium hexafluorophosphate (1.50 equiv., 1341 mg, 3.53 mmol) and (2,6-dioxo-3-piperidyl)ammonium chloride (1.00 equiv., 387 mg, 2.35 mmol) was added in a single portion to the reaction mixture and then stirred at ambient temperature overnight. The reaction was then monitored by TLC until no more starting material could be observed, which upon completion was concentrated under reduced pressure and purified by reverse-phase chromatography (formic acid 0.1% in water/ACN). The desired fractions was then collected and concentrated under reduced pressure and taken up in ice-cold water and filtered. The filter cake was then washed with 10 mL of ice-cold water and then dried to give N-(2,6-dioxo-3-piperidyl)-2-fluoro-4-(hydroxymethyl)benzamide (255 mg, 0.910 mmol, 39% yield) as an off-white solid.

LCMS: 343.15 [M+Na⁺]: 366.

¹H NMR (500 MHz, DMSO-d₆): δ 10.86 (s, 1H), 8.49 (dd, J=8.2, 3.5 Hz, 1H), 7.65 (t, J=7.9 Hz, 1H), 7.29-7.17 (m, 2H), 5.44 (t, J=5.8 Hz, 1H), 4.81-4.71 (m, 1H), 4.56 (d, J=5.8 Hz, 2H), 2.82-2.72 (m, 1H), 2.55 (t, J=3.0 Hz, 1H), 2.15-2.04 (m, 1H), 2.06-1.98 (m, 1H).

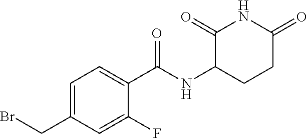 [SMILES: O=C1CCC(NC(=O)c2ccc(CBr)cc2F)C(=O)N1]

Compound 28: 4-(Bromomethyl)-N-(2,6-dioxo-3-piperidyl)-2-fluoro-benzamide: To a solution of N-(2,6-dioxo-3-piperidyl)-2-fluoro-4-(hydroxymethyl)benzamide (1.00 equiv., 200 mg, 0.714 mmol) in dry DCM (4.8 mL) was added phosphorus dihydride tribromide (2.00 equiv., 135 μL, 1.43 mmol) at 0° C. and stirred. The reaction was then slowly warmed to ambient temperature and stirred overnight. The reaction was then quenched with saturated NaHCO₃ solution and extracted with DCM. The organics were then combined and dried with MgSO₄ before concentration under reduced pressure to give 4-(bromomethyl)-N-(2,6-dioxo-3-piperidyl)-2-fluoro-benzamide (190 mg, 0.554 mmol, 78% yield) as a white solid.

LCMS: 343.15 [M+Na⁺]: 366.

¹H NMR (500 MHz, DMSO-d₆): δ 10.88 (s, 1H), 8.63 (dd, J=8.3, 2.5 Hz, 1H), 7.65 (t, J=7.7 Hz, 1H), 7.43 (dd, J=11.2, 1.6 Hz, 1H), 7.39 (dd, J=7.9, 1.6 Hz, 1H), 4.80-4.75 (m, 1H), 4.74 (s, 2H), 2.84-2.73 (m, 1H), 2.55 (t, J=3.8 Hz, 1H), 2.15-2.04 (m, 1H), 2.04-1.98 (m, 1H).

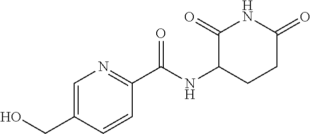 [SMILES: O=C1CCC(NC(=O)c2ccc(CO)cn2)C(=O)N1]

Compound 29: N-(2,6-Dioxo-3-piperidyl)-5-(hydroxymethyl)pyridine-2-carboxamide: A solution of (2,6-dioxo-3-piperidyl)ammonium chloride (1.25 equiv., 672 mg, 4.08 mmol) and 5-(hydroxymethyl)pyridine-2-carboxylic acid (1.00 equiv., 500 mg, 3.26 mmol) in DMF (20.4 mL) was treated with [dimethylamino(triazolo[4,5-b]pyridin-3-yloxy)methylene]-dimethyl-ammonium hexafluorophosphate (1.75 equiv., 2173 mg, 5.71 mmol) and DIPEA (5.00 equiv., 2.8 mL, 16.3 mmol). The reaction was then stirred at ambient temperature overnight. The reaction was then analysed by LC-MS which indicated full consumption of starting material. Thereafter, the reaction was concentrated under reduced pressure and subjected to SCX-column chromatography. The eluant from the SCX-column was then additionally purified by reverse-phase chromatography (formic acid 0.1% in water/ACN) to give N-(2,6-dioxo-3-piperidyl)-5-(hydroxymethyl)pyridine-2-carboxamide (363 mg, 1.38 mmol, 42% yield) as a brown solid. The compound was then taken into the next step without further purification.

LCMS: 263.25 [M+H⁺]: 264; [M+H-]: 262.

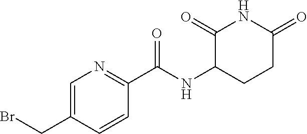 [SMILES: O=C1CCC(NC(=O)c2ccc(CBr)cn2)C(=O)N1]

Compound 30: 5-(Bromomethyl)-N-(2,6-dioxo-3-piperidyl)pyridine-2-carboxamide: To a solution of N-(2,6-dioxo-3-piperidyl)-5-(hydroxymethyl)pyridine-2-carboxamide (1.00 equiv., 340 mg, 1.29 mmol) in THE (32.3 mL) was added carbon tetrabromide (3.00 equiv., 1285 mg, 3.87 mmol) and triphenylphosphane (2.00 equiv., 678 mg, 2.58 mmol) and stirred at ambient temperature for 30 minutes. Thereafter, the reaction was analysed by LC-MS which indicated full consumption of the starting material. Thereafter, the reaction was concentrated under reduced pressure and taken up in ice-cold EtOAc which was then filtered. The filter-cake was dried and then purified by normal-phase chromatography eluting in 30% DCM/acetone. The desired fractions were concentrated under reduced pressure give 5-(bromomethyl)-N-(2,6-dioxo-3-piperidyl)pyridine-2-carboxamide (87 mg, 0.267 mmol, 21% yield) as a white solid.

LCMS: 326.15 [M+H⁺]: 326.

¹H NMR (500 MHz, DMSO-d₆): δ 10.87 (s, 1H), 9.10 (d, J=8.5 Hz, 1H), 8.75 (s, 1H), 8.13-8.02 (m, 2H), 4.84 (s, 2H), 4.83-4.75 (m, 1H), 2.86-2.76 (m, 1H), 2.57-2.52 (m, 1H), 2.29-2.17 (m, 1H), 2.03-1.97 (m, 1H)

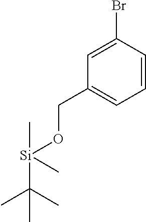 [SMILES: CC(C)(C)[Si](C)(C)OCc1cccc(Br)c1]

Compound 31: (3-Bromophenyl)methoxy-tert-butyl-dimethyl-silane: Synthesised according to the procedure described in CN117343081 A.

¹H NMR (500 MHz, CDCl₃): δ 7.40 (s, 1H), 7.31-7.28 (m, 1H), 7.18-7.08 (m, 2H), 4.65 (s, 2H), 0.89 (s, 9H), 0.05 (s, 6H).

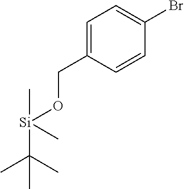 [SMILES: CC(C)(C)[Si](C)(C)OCc1ccc(Br)cc1]

Compound 32: (4-Bromophenyl)methoxy-tert-butyl-dimethyl-silane: Synthesised according to the procedure described in ACS Catalysis, 2024, 14, 4675.

¹H NMR (500 MHz, CDCl₃): δ 7.45 (d, J=8.5 Hz, 2H), 7.21 (d, J=8.6 Hz, 2H), 4.69 (s, 1H), 0.95 (s, 9H), 0.10 (s, 6H).

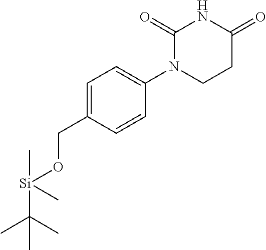 [SMILES: CC(C)(C)[Si](C)(C)OCc1ccc(N2CCC(=O)NC2=O)cc1]

Compound 33: 1-[4-[[Tert-butyl(dimethyl)silyl]oxymethyl]phenyl]hexahydropyrimidine-2,4-dione: To a degassed microwave vial was added (4-bromophenyl)methoxy-tert-butyl-dimethyl-silane (1.00 equiv., 500 mg, 1.66 mmol), hexahydropyrimidine-2,4-dione (4.50 equiv., 852 mg, 7.47 mmol) and caesium carbonate (3.00 equiv., 1622 mg, 4.98 mmol) in 1,4-dioxane (16.6 mL). The mixture was then degassed for 15 minutes followed by the addition of BrettPhos Pd G3 (0.100 equiv., 150 mg, 0.166 mmol) and BrettPhos (0.100 equiv., 89 mg, 0.166 mmol). The reaction mixture was then evacuated and refilled with nitrogen three times before heating to 100° C. overnight. The reaction mixture was then diluted in 50 mL of water and extracted twice with 40 mL of EtOAc. The organics were then separated and dried with MgSO₄ before concentration under reduced pressure. The crude was then purified by flash column chromatography eluting 0-100% EtOAc in Hp. The desired fractions were concentrated under reduced pressure give 1-[4-[[tert-butyl(dimethyl)silyl]oxymethyl]phenyl]hexahydropyrimidine-2,4-dione (310 mg, 0.927 mmol, 56% yield) as a colourless oil.

LCMS: 334.17 [M+H⁺]: 335.1.

¹H NMR (500 MHz, CDCl₃): δ 7.37 (d, J=8.4 Hz, 2H), 7.28-7.23 (m, 2H), 4.74 (s, 2H), 3.87 (t, J=6.7 Hz, 2H), 2.83 (t, J=6.7 Hz, 2H), 0.95 (s, 9H), 0.11 (s, 6H).

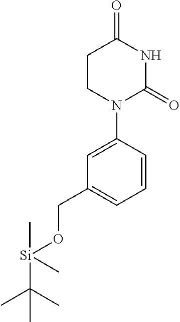 [SMILES: CC(C)(C)[Si](C)(C)OCc1cccc(N2CCC(=O)NC2=O)c1]

Compound 34: 1-[3-[[Tert-butyl(dimethyl)silyl]oxymethyl]phenyl]hexahydropyrimidine-2,4-dione: To a degassed microwave vial was added (3-bromophenyl)methoxy-tert-butyl-dimethyl-silane (1.00 equiv., 500 mg, 1.66 mmol), hexahydropyrimidine-2,4-dione (4.50 equiv., 852 mg, 7.47 mmol) and caesium carbonate (3.00 equiv., 1622 mg, 4.98 mmol) in 1,4-dioxane (16.6 mL). The reaction vessel was then evacuated and backfilled with nitrogen three times. Then BrettPhos Pd G3 (0.100 equiv., 150 mg, 0.166 mmol) and BrettPhos (0.100 equiv., 89 mg, 0.166 mmol) was added, then the reaction vessel was sealed. The reaction was then heated at 100° C. overnight. The reaction mixture was then diluted in 15 mL of water and extracted twice with 20 mL of EtOAc. The organics were then washed once with saturated brine solution, then collected and dried with MgSO₄ before concentration under reduced pressure. The crude was then purified by flash column chromatography eluting in EtOAc in Hp. The desired fractions were concentrated under reduced pressure give 1-[3-[[tert-butyl(dimethyl)silyl]oxymethyl]phenyl]hexahydropyrimidine-2,4-dione (335 mg, 1.00 mmol, 60% yield) as a colourless oil.

¹H NMR (500 MHz, CDCl₃): δ 7.40-7.35 (m, 2H), 7.24 (d, J=7.6 Hz, 1H), 7.19 (d, J=7.9 Hz, 1H), 4.75 (s, 2H), 3.88 (t, J=6.7 Hz, 2H), 2.83 (t, J=6.6 Hz, 2H), 0.95 (s, 9H), 0.11 (s, 6H).

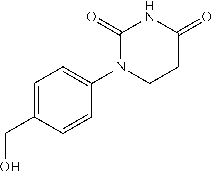 [SMILES: O=C1CCN(c2ccc(CO)cc2)C(=O)N1]

Compound 35: 1-(4-(Hydroxymethyl)phenyl)dihydropyrimidine-2,4(1H,3H)-dione: To a solution of 1-[4-[[tert-butyl(dimethyl)silyl]oxymethyl]phenyl]hexahydropyrimidine-2,4-dione (1.00 equiv., 496 mg, 1.48 mmol) in THE (9 mL) was added tetrabutylammonium fluoride (1.70 equiv., 2.5 mL, 2.52 mmol) and stirred at ambient temperature for 1 h. Thereafter, the reaction mixture was concentrated under reduced pressure and purified by flash chromatography eluting in 20% DCM in MeOH to give 1-[4-(hydroxymethyl)phenyl]hexahydropyrimidine-2,4-dione (302 mg, 1.37 mmol, 93% yield) as a light yellow solid.

LCMS: 220.08, [M+H⁺]: 220.9.

¹H NMR (500 MHz, DMSO-d₆): δ 10.34 (s, 1H), 7.32 (d, J=8.5 Hz, 2H), 7.27 (d, J=8.4 Hz, 2H), 5.20 (t, J=5.7 Hz, 1H), 4.49 (d, J=5.7 Hz, 2H), 3.77 (t, J=6.6 Hz, 2H), 2.70 (t, J=6.7 Hz, 2H).

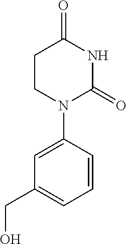 [SMILES: O=C1CCN(c2cccc(CO)c2)C(=O)N1]

Compound 36: 1-[3-(Hydroxymethyl)phenyl]hexahydropyrimidine-2,4-dione: To a solution of 1-[3-[[tert-butyl(dimethyl)silyl]oxymethyl]phenyl]hexahydropyrimidine-2,4-dione (1.00 equiv., 335 mg, 1.00 mmol) in THE (6.26 mL) was added tetrabutylammonium fluoride (1.70 equiv., 1.7 mL, 1.70 mmol) and stirred at ambient temperature for 1 h. Thereafter, the reaction mixture was concentrated under reduced pressure and purified by flash chromatography eluting in 20% DCM in MeOH to give 1-[3-(hydroxymethyl)phenyl]hexahydropyrimidine-2,4-dione (197 mg, 0.895 mmol, 89% yield) as a light yellow solid.

¹H NMR (500 MHz, DMSO-d₆): δ 10.34 (s, 1H), 7.33 (t, J=7.7 Hz, 1H), 7.27 (d, J=2.0 Hz, 1H), 7.21-7.16 (m, 2H), 5.24 (t, J=5.7 Hz, 1H), 4.50 (d, J=5.7 Hz, 2H), 3.77 (t, J=6.6 Hz, 2H), 2.70 (t, J=6.6 Hz, 2H).

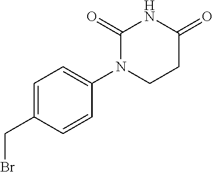 [SMILES: O=C1CCN(c2ccc(CBr)cc2)C(=O)N1]

Compound 37: 1-[4-(Bromomethyl)phenyl]hexahydropyrimidine-2,4-dione: To a solution of hydrobromic acid solution (40% in AcOH, 3.7 mL) was added 1-[4-(hydroxymethyl)phenyl]hexahydropyrimidine-2,4-dione (1.00 equiv., 115 mg, 0.522 mmol) and then stirred at 90° C. for 1 h. Thereafter, the reaction was monitored by LCMS which indicated full consumption of starting material and formation of product. The reaction was then taken up in distilled water (10 mL) and washed with EtOAc (3×10 mL). The organic layers were then dried with MgSO₄ and concentrated under reduced pressure to give 1-[4-(bromomethyl)phenyl]hexahydropyrimidine-2,4-dione (83 mg, 0.292 mmol, 56% yield) as a brown solid.

LCMS: 283.12 [M+H⁺]: 283.0.

¹H NMR (500 MHz, DMSO-d₆): δ 10.42 (s, 1H), 7.46 (d, J=8.2 Hz, 2H), 7.32 (d, J=8.6 Hz, 2H), 4.72 (s, 2H), 3.80 (t, J=6.7 Hz, 2H), 2.70 (t, J=6.6 Hz, 2H).

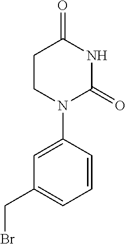 [SMILES: O=C1CCN(c2cccc(CBr)c2)C(=O)N1]

Compound 38: 1-[3-(Bromomethyl)phenyl]hexahydropyrimidine-2,4-dione: To a solution of hydrobromic acid solution (40% in acetic acid, 4.8 mL) was added 1-[3-(hydroxymethyl)phenyl]hexahydropyrimidine-2,4-dione (1.00 equiv., 150 mg, 0.681 mmol) and then stirred at 90° C. for 1 h. Thereafter, the reaction was monitored by LCMS which indicated full consumption of starting material and formation of product. The reaction was then taken up in distilled water (10 mL) and washed with EtOAc (3×10 mL). The organic layers were the dried with MgSO₄ and concentrated under reduced pressure to give 1-[3-(bromomethyl)phenyl]hexahydropyrimidine-2,4-dione (125 mg, 0.442 mmol, 65% yield) as a brown solid.

LCMS: 283.12 [M+H⁺]: 283.0.

¹H NMR (500 MHz, DMSO-d₆): δ 10.41 (s, 1H), 7.42 (s, 1H), 7.38 (t, J=7.8 Hz, 1H), 7.33-7.26 (m, 2H), 4.71 (s, 2H), 3.79 (t, J=6.6 Hz, 2H), 2.71 (t, J=6.7 Hz, 2H).

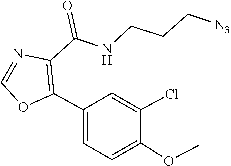 [SMILES: COc1ccc(-c2ocnc2C(=O)NCCCN=[N+]=[N-])cc1Cl]

Compound 39: N-(3-Azidopropyl)-5-(3-chloro-4-methoxyphenyl)oxazole-4-carboxamide: Synthesised according to the procedure described in Eur. J. Med. Chem., 2021, 226, 113889.

¹H NMR (500 MHz, CDCl₃): δ 8.37 (dd, J=8.7, 2.2 Hz, 1H), 8.32 (d, J=2.2 Hz, 1H), 7.80 (s, 1H), 7.40 (s, 1H), 7.00 (d, J=8.8 Hz, 1H), 3.96 (s, 3H), 3.54 (q, J=6.6 Hz, 2H), 3.43 (t, J=6.6 Hz, 2H), 1.92 (p, J=6.7 Hz, 2H).

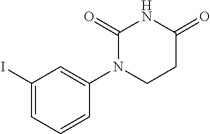 [SMILES: O=C1CCN(c2cccc(I)c2)C(=O)N1]

Compound 40: 1-(3-Iodophenyl)dihydropyrimidine-2,4(1H,3H)-dione: To a solution of 3-iodoaniline (2.6 mL, 22.8 mmol) in 40 mL of toluene was added acrylic acid (12.0 mL, 29.64 mmol). The mixture was stirred at 110° C. overnight. The solvent was removed to obtain 6.64 g of an orange oil. The orange oil was taken up in 55 mL of AcOH and to this was added urea (4.1 g, 68.4 mmol). The mixture was stirred at 120° C. over a weekend. The mixture was neutralized with NaHCO₃ and the reaction extracted with EtOAc. The combined organic layers were dried with MgSO₄, filtered and the solvent was concentrated under reduced pressure. EtOAc was added and the suspension was sonicated and filtered to isolate the desired product (3.4 g, 47% yield) as a solid.

¹H NMR (500 MHz, DMSO-d₆): δ 10.42 (s, 1H), 7.73 (s, 1H), 7.59 (d, J=7.8 Hz, 1H), 7.35 (d, J=8.0 Hz, 1H), 7.19 (t, J=8.0 Hz, 1H), 3.78 (t, J=6.6 Hz, 2H), 2.69 (t, J=6.6 Hz, 2H).

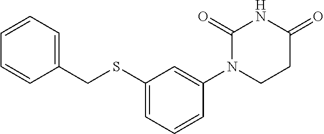 [SMILES: O=C1CCN(c2cccc(SCc3ccccc3)c2)C(=O)N1]

Compound 41: 1-(3-(Benzylthio)phenyl)dihydropyrimidine-2,4(1H,3H)-dione: 1-(3-Iodophenyl)dihydropyrimidine-2,4(1H, 3H)-dione (600 mg, 1.9 mmol), phenylmethanethiol (223 μL, 1.9 mmol), DIPEA (993 μL, 5.7 mmol) and 5 mL of 1,4-dioxane were added to a flask and placed under nitrogen. 1,1′-(9,9-Dimethyl-9H-xanthene-4,5-diyl)bis[1,1-diphenylphosphine](110 mg, 0.19 mmol) and Pd₂(dba)₃ (87 mg, 0.095 mmol) were then added. The flask was heated at 100° C. for 1 h. The reaction was then filtered through Celite and washed with EtOAc before concentration. Purification was then performed on the resulting residue using Hp/EtOAc as the eluent to obtain the desired product (563 mg, 95% yield).

¹H NMR (500 MHz, DMSO-d₆): δ 10.38 (s, 1H), 7.37 (d, J=7.4 Hz, 2H), 7.33-7.27 (m, 4H), 7.23 (t, J=7.3 Hz, 1H), 7.18 (d, J=7.9 Hz, 1H), 7.15-7.11 (m, 1H), 4.25 (s, 2H), 3.74 (t, J=6.6 Hz, 2H), 2.69 (t, J=6.6 Hz, 2H).

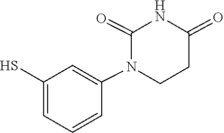 [SMILES: O=C1CCN(c2cccc(S)c2)C(=O)N1]

Compound 42: 1-(3-Mercaptophenyl)dihydropyrimidine-2,4(1H,3H)-dione: 1-(3-(Benzylthio)phenyl)dihydropyrimidine-2,4(1H,3H)-dione (1.8 g, 5.76 mmol) was suspended in 72 mL of toluene under a nitrogen atmosphere and AlCl₃ (3.8 g, 28.8 mmol) was added. The reaction was stirred at RT for 20 minutes. The reaction was then diluted with EtOAc and the AlCl₃ was quenched with ice/water. The organic layer was washed with water, dried with MgSO₄ and filtered. The solvent was concentrated under reduced pressure and the product was purified by reverse phase chromatography (formic acid 0.1% in water/ACN) to obtain a white solid (579 mg, 45% yield).

¹H NMR (500 MHz, DMSO-d₆): δ 10.38 (s, 1H), 7.30-7.23 (m, 2H), 7.15 (d, J=7.8 Hz, 1H), 7.08 (d, J=8.0 Hz, 1H), 5.53 (s, 1H), 3.75 (t, J=6.7 Hz, 2H), 2.69 (t, J=6.7 Hz, 2H).

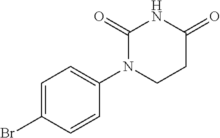 [SMILES: O=C1CCN(c2ccc(Br)cc2)C(=O)N1]

Compound 43: 1-(4-Bromophenyl)dihydropyrimidine-2,4(1H,3H)-dione: A solution of 4-bromoaniline (2 g, 11.6 mmol) and acrylic acid (3.18 mL, 46.4 mmol) was stirred at 110° C. for 3 h. Then urea (2.8 g, 46.4 mmol) and AcOH (20 mL) were added and the mixture was stirred at 120° C. for 12 h. On completion, the reaction was poured into water (50 mL) at 0° C. The solid was filtered, and the filter cake was washed with water and concentrated under reduced pressure. EtOAc was added and the mixture filtered to isolate the product as a brown solid (1.7 g, 55% yield).

¹H NMR (500 MHz, DMSO-d₆): δ 10.42 (s, 1H), 7.57 (d, J=8.7 Hz, 2H), 7.30 (d, J=8.7 Hz, 2H), 3.78 (t, J=6.6 Hz, 2H), 2.70 (t, J=6.6 Hz, 2H).

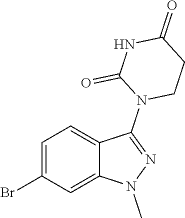 [SMILES: Cn1nc(N2CCC(=O)NC2=O)c2ccc(Br)cc21]

Compound 44: 1-(6-Bromo-1-methyl-1H-indazol-3-yl)dihydropyrimidine-2,4(1H, 3H)-dione: Synthesised according to the procedure described in WO2024030968 A1.

¹H NMR (500 MHz, DMSO-d₆): δ 10.59 (s, 1H), 7.97 (d, J=1.0 Hz, 1H), 7.62 (d, J=8.7 Hz, 1H), 7.25 (dd, J=8.7, 1.5 Hz, 1H), 3.98 (s, 3H), 3.93 (t, J=6.7 Hz, 2H), 2.76 (t, J=6.7 Hz, 2H).

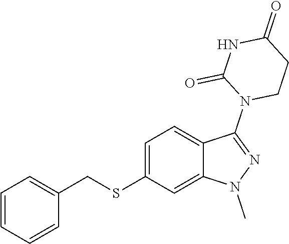 [SMILES: Cn1nc(N2CCC(=O)NC2=O)c2ccc(SCc3ccccc3)cc21]

Compound 45: 1-(6-(Benzylthio)-1-methyl-1H-indazol-3-yl)dihydropyrimidine-2,4(1H, 3H)-dione: 1-(6-Bromo-1-methyl-1H-indazol-3-yl)dihydropyrimidine-2,4(1H, 3H)-dione (300 mg, 0.93 mmol), phenylmethanethiol (109 μL, 0.93 mmol), DIPEA (486 μL, 2.79 mmol) and 1.4-dioxane (1.95 mL) were added to a flask and placed under a nitrogen atmosphere. 1,1′-(9,9-Dimethyl-9H-xanthene-4,5-diyl)bis[1,1-diphenylphosphine](54 mg, 0.093 mmol) and Pd₂(dba)₃ (43 mg, 0.046 mmol) were then added. The reaction was heated at 100° C. for 1 h. Once the reaction was complete it was filtered through Celite and washed with EtOAc before concentrating the filtrate. The resultant residue was purified by silica chromatography using Hp/EtOAc/MeOH as the eluent to give the product (340 mg, quantitative yield).

¹H NMR (500 MHz, DMSO-d₆): δ 10.55 (s, 1H), 7.57 (s, 1H), 7.55 (d, J=8.6 Hz, 1H), 7.41 (d, J=7.4 Hz, 2H), 7.30 (t, J=7.5 Hz, 2H), 7.23 (t, J=7.3 Hz, 1H), 7.07 (dd, J=8.6, 1.2 Hz, 1H), 4.36 (s, 2H), 3.94 (s, 3H), 3.91 (t, J=6.7 Hz, 2H), 2.74 (t, J=6.7 Hz, 2H).

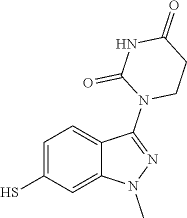 [SMILES: Cn1nc(N2CCC(=O)NC2=O)c2ccc(S)cc21]

Compound 46: 1-(6-Bromo-1-methyl-1H-indazol-3-yl)dihydropyrimidine-2,4(1H, 3H)-dione: Aluminium chloride (164 mg, 1.23 mmol) was added to a suspension of 1-(6-(benzylthio)-1-methyl-1H-indazol-3-yl)dihydropyrimidine-2,4(1H, 3H)-dione (100 mg, 0.27 mmol) in 4 mL of toluene under a nitrogen atmosphere. Once the reaction was complete it was quenched with ice/water and extracted with EtOAc. The organic layer was dried with MgSO₄, filtered and the solvent was concentrated under reduced pressure. The crude residue was purified by reverse phase chromatography to afford the product (45 mg, 60%).

¹H NMR (500 MHz, DMSO-d₆): δ 10.55 (s, 1H), 7.54-7.51 (m, 2H), 7.02 (dd, J=8.7, 1.0 Hz, 1H), 5.60 (s, 1H), 3.95-3.87 (m, 5H), 2.75 (t, J=6.7 Hz, 2H).

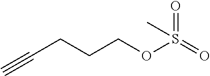

Compound 47: Pent-4-yn-1-yl methanesulfonate: Synthesised according to the procedure described in J. Brazil. Chem. Soc., 2023, 34, 1810.

¹H NMR (500 MHz, CDCl₃): δ 4.35 (t, J=6.1 Hz, 2H), 3.02 (s, 3H), 2.36 (td, J=6.8, 2.6 Hz, 2H), 2.01 (t, J=2.6 Hz, 1H), 2.00-1.93 (m, 2H).

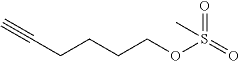

Compound 48: Hex-5-yn-1-yl methanesulfonate: Synthesised according to the procedure described in J. Med. Chem., 2022, 65, 4649.

¹H NMR (500 MHz, DMSO-d₆): δ 4.22 (t, J=6.4 Hz, 2H), 3.17 (s, 3H), 2.80 (t, J=2.7 Hz, 1H), 2.24-2.18 (m, 2H), 1.78-1.72 (m, 2H), 1.57-1.49 (m, 2H).

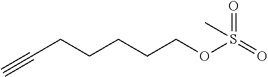

Compound 49: Hept-6-yn-1-yl methanesulfonate: Synthesised according to the procedure described in WO2022236058 A1.

¹H NMR (500 MHz, CDCl₃): δ 4.24 (t, J=6.5 Hz, 2H), 3.01 (s, 3H), 2.22 (td, J=6.7, 2.6 Hz, 2H), 1.95 (t, J=2.7 Hz, 1H), 1.82-1.74 (m, 2H), 1.63-1.49 (m, 4H).

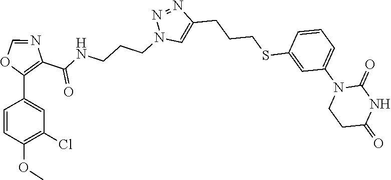 [SMILES: COc1ccc(-c2ocnc2C(=O)NCCCn2cc(CCCSc3cccc(N4CCC(=O)NC4=O)c3)nn2)cc1Cl]

Compound 50: 5-(3-Chloro-4-methoxyphenyl)-N-(3-(4-(3-((3-(2,4-dioxotetrahydropyrimidin-1(2H)-yl)phenyl)thio)propyl)-1H-1,2,3-triazol-1-yl)propyl)oxazole-4-carboxamide: Synthesised according to Method C.

LCMS: 623.18 [M+H⁺]: 624.2.

¹H NMR (500 MHz, CDCl₃): δ 8.86 (s, 1H), 8.35-8.30 (m, 2H), 7.99 (s, 1H), 7.74 (t, J=6.2 Hz, 1H), 7.33-7.26 (m, 3H), 7.10-7.06 (m, 1H), 7.03-6.99 (m, 1H), 4.39 (t, J=6.7 Hz, 2H), 3.96 (s, 3H), 3.88 (t, J=6.7 Hz, 2H), 3.42 (q, J=6.4 Hz, 2H), 2.93 (t, J=7.1 Hz, 2H), 2.89-2.82 (m, 4H), 2.24-2.15 (m, 2H), 2.10-1.98 (m, 2H).

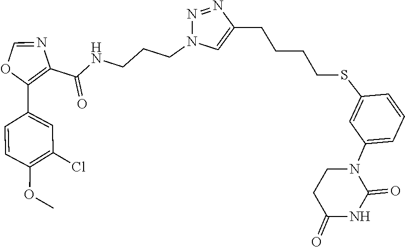 [SMILES: COc1ccc(-c2ocnc2C(=O)NCCCn2cc(CCCCSc3cccc(N4CCC(=O)NC4=O)c3)nn2)cc1Cl]

Compound 51: 5-(3-Chloro-4-methoxyphenyl)-N-(3-(4-(4-((3-(2,4-dioxotetrahydropyrimidin-1(2H)-yl)phenyl)thio)butyl)-1H-1,2,3-triazol-1-yl)propyl)oxazole-4-carboxamide: Synthesised according to Method C.

LCMS: 637.19 [M+H⁺]: 638.2.

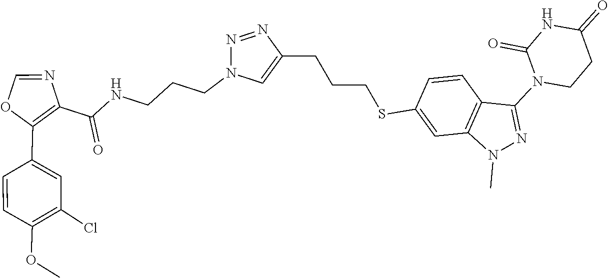 [SMILES: COc1ccc(-c2ocnc2C(=O)NCCCn2cc(CCCSc3ccc4c(N5CCC(=O)NC5=O)nn(C)c4c3)nn2)cc1Cl]

Compound 52: 5-(3-Chloro-4-methoxyphenyl)-N-(3-(4-(5-((3-(2,4-dioxotetrahydropyrimidin-1(2H)-yl)phenyl)thio)pentyl)-1H-1,2,3-triazol-1-yl)propyl)oxazole-4-carboxamide: Synthesised according to Method C.

LCMS: 651.20 [M+H⁺]: 652.3

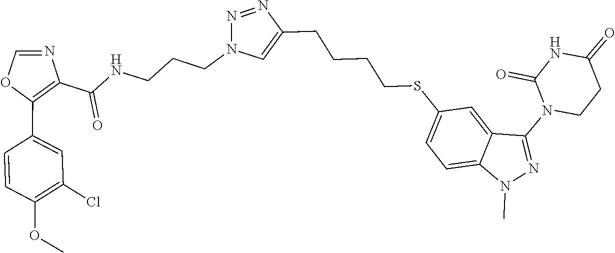 [SMILES: COc1ccc(-c2ocnc2C(=O)NCCCn2cc(CCCCSc3ccc4c(c3)c(N3CCC(=O)NC3=O)nn4C)nn2)cc1Cl]

Compound 53: 5-(3-Chloro-4-methoxyphenyl)-N-(3-(4-(3-((3-(2,4-dioxotetrahydropyrimidin-1(2H)-yl)-1-methyl-1H-indazol-6-yl)thio)propyl)-1H-1,2,3-triazol-1-yl)propyl)oxazole-4-carboxamide: Synthesised according to Method C.

LCMS: 691.21 [M+H⁺]: 692.3.

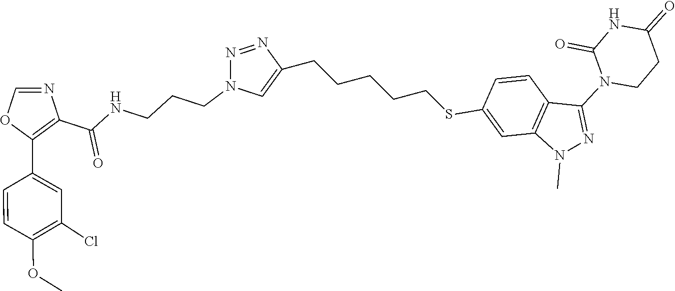 [SMILES: COc1ccc(-c2ocnc2C(=O)NCCCn2cc(CCCCCSc3ccc4c(N5CCC(=O)NC5=O)nn(C)c4c3)nn2)cc1Cl]

Compound 54: 5-(3-Chloro-4-methoxyphenyl)-N-(3-(4-(5-((3-(2,4-dioxotetrahydropyrimidin-1(2H)-yl)-1-methyl-1H-indazol-6-yl)thio)pentyl)-1H-1,2,3-triazol-1-yl)propyl)oxazole-4-carboxamide: Synthesised according to Method C.

LCMS: 705.22 [M+H⁺]: 706.2.

### Results

The present investigators have expanded on the existing direct-to-biology (D2B) methods of forming bifunctional compounds. As described above, a bifunctional compound can typically be considered to comprise three parts; a linker, a first ligand, and a second ligand. In many existing D2B methods, due to limitations in the design of linkers or ligand molecules, the resultant structural diversity of the bifunctional compounds is limited. This may be due to, for example, the linker comprising only one reactive moiety, to overcome issues with competing or incompatible reactive groups.

Additionally, some existing D2B methods are operatively complex, for example, comprising 3 or more chemical synthesis steps. It is challenging to develop operatively simple D2B methods that enable high structural diversity, as achieving one benchmark is often at detriment to achieving the other, as more synthesis steps typically results in greater structural diversity. However, the present investigators have identified operatively simple (D2B) methods with high levels of structural diversity in the resultant bifunctional compounds.

This has been achieved through the clever design and use of linkers comprising orthogonally reactive moieties, paired with complementary moieties comprised within molecules selected to react with the orthogonally reactive moieties. The methods disclosed herein are “one-pot” methods, thus enabling an array of linkers and molecules to be run on, for example, a single multi-well plate to generate a large and diverse library of bifunctional compounds. Each well of the plate may have a different combination of reagents, and may then be analysed for formation of a bifunctional compound, and subsequent biological testing of any formed bifunctional compound. Hence, the method is suitable for screening reagents to assess their suitability in forming bifunctional compounds.

The present investigators have identified three general orthogonal platforms of the methods disclosed herein.

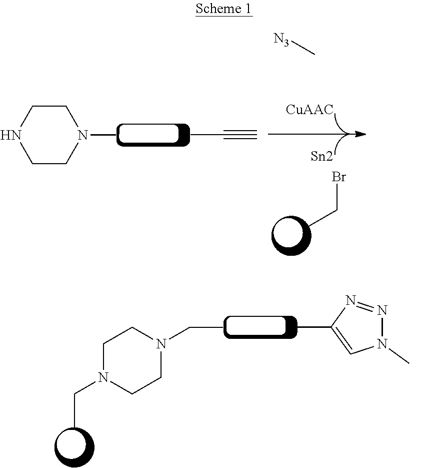

Scheme 1 shows a general scheme of a linker comprising an amine (piperidine depicted) and an alkyne (ethyne depicted) as orthogonal groups; a nucleofuge moiety (bromide depicted) can react with the amine in an S_{N}2 reaction; and an azide moiety can react with the alkyne in a copper-catalysed azide-alkyne cycloaddition (CuAAC) reaction.

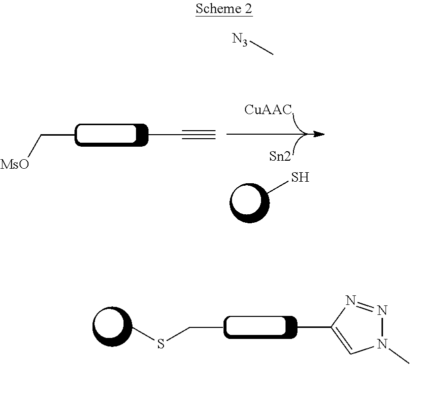

Scheme 2 shows a general scheme of a linker comprising a nucleofuge (mesylate depicted) and an alkyne as orthogonal groups; a nucleophile (thiol depicted) can react with the nucleofuge in an S_{N}2 reaction; and an azide can react with the alkyne in a CuAAC reaction.

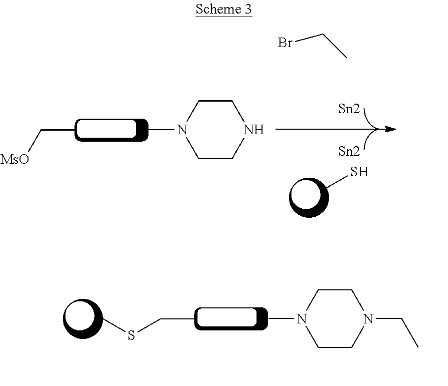

Scheme 3 shows a general scheme of a linker comprising a nucleofuge (mesylate depicted) and an amine (piperidine depicted) as orthogonal groups; a nucleofuge (bromide depicted) can react with the amine in an S_{N}2 reaction; and the nucleofuge of the linker can react with a nucleophile (thiol depicted) also in an S_{N}2 reaction. Without being bound by theory, these reactive moieties are compatible due to the significantly greater rate of chemical reaction of (i) the nucleofuge of the linker reacting with a molecule comprising a nucleophile, relative to (ii) the amine of the linker reacting with a molecule comprising a nucleofuge. This difference in the rate of reaction is particularly evident when, for example, (i) a sulfonate is reacted with a thiol, relative to (ii) a piperidine is reacted with a bromide. This is evidenced by the exemplary methods described above, whereby Method C comprises an S_{N}2 reaction of a sulfonate with a thiol which is carried out for just 30 minutes.

Furthermore, an amine group of the general linkers depicted in Scheme 1 and Scheme 2 can react in other reactions known to the skilled person, such as in an S_{N}Ar reaction with an electropositive (hetero)aryl nucleofuge; a C—N cross-coupling reaction with a (hetero)aryl nucleofuge; an amide coupling reaction with an activated carboxylic acid, carboxylic acid, or acyl chloride; and a substitution reaction with a sulfonyl chloride to form a sulfonamide. These reactions are described in further detail above.

Each reaction described above is selective and efficient. That is to say, the reactive moieties have been selected so as to not be incompatible nor in competition with one another. This has then been tested and demonstrated by extensive experimental work described above and in the methods described herein.

To develop the methods of the disclosure, the present investigators developed and identified a large variety of linkers comprising orthogonally reactive moieties—the chemical structure, synthetic procedure, and characterisation data of exemplary linkers is described above, particularly for compounds 2, 4, 6, 7, 9-14, 16, 17, 19, and 47-49.

Furthermore, some linkers disclosed herein have been designed to reduce operative complexity, specifically purification complexity: the linkers may comprise a basic amine group. The investigators have found that linkers comprising such a group can be used to manufacture bifunctional compounds more amenable to purification, particularly high-throughput purification, by exploiting the basicity of the group via ion-exchange purification systems, such as cation-exchange. That is to say, the basic amine group may bind to a cation-exchange system, allowing unbound impurities (such as by-products, reagents, catalysts, and/or unreacted starting materials) to be washed away with an organic solvent—the bound amine group may then be eluted by adjusting the pH of the cation-exchange system, often with a solution of ammonium hydroxide. Cation-exchange systems, like other purification systems, are available in a plate-based format, and thus are particularly compatible with the D2B, plate-based methods of the present disclosure, often enabling direct and simple loading of crude reaction material. Specific protocols for plate-based purification are described above, particularly in Method A and Method B. It is useful to note that, whilst these features are useful, they are not required to carry out the methods disclosed herein.

Additionally, the present investigators have developed and identified a large variety of binders (or molecules, or ligands) for use in the methods disclosed herein, wherein the investigators have identified and incorporated a suitable moiety in the binder to react with one of the two orthogonally reactive moieties—the chemical structure, synthetic procedure, and characterisation data of exemplary binders is described above, particularly compounds 26, 28, 30, 37, and 38.

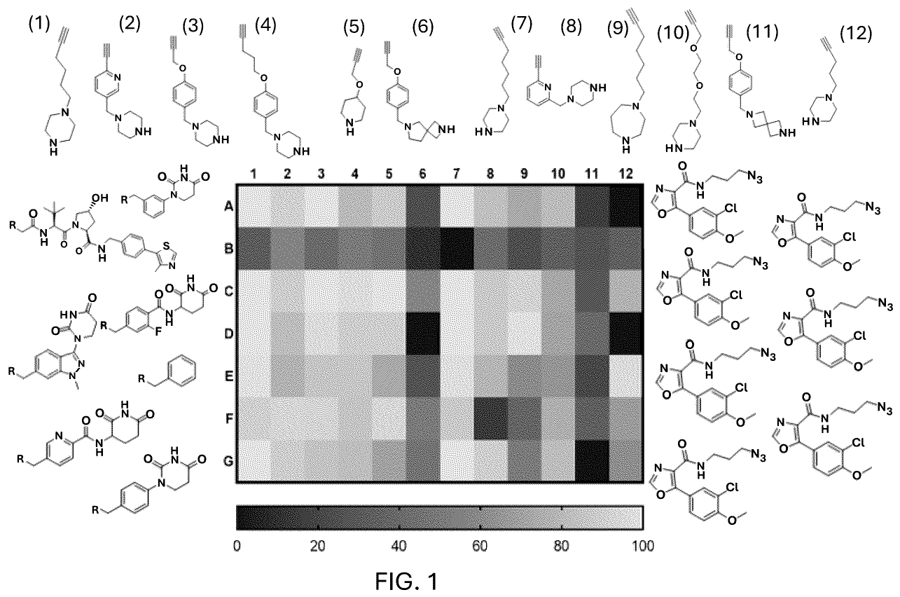

FIG. 1 shows the results of a D2B method of the present disclosure, utilising the general orthogonal platform depicted in Scheme 1, and thus general experimental Method A described above. Method B could also be used. The plot is representative of an array run on a plate, with each well of the plate comprising different reagents in an operatively simple two-step process. The wide variety of reagents, and flexibility of choice of reagents, demonstrates the potential for high structural diversity of the methods presented herein. For example, well ‘A1’ (top left) comprises compounds 14, 38, and 39. ‘Row E’ of FIG. 1 comprises the negative control molecule, benzyl bromide, which is used to verify that the reaction of the molecules and linkers of the array is successful. The bar at the bottom of FIG. 1 relates to the purity of the resultant bifunctional compound at the end of the process, as measured by UV-vis spectroscopy (in an LCMS system)—a darker well indicates lower purity, a lighter well indicates higher purity, thus FIG. 1 shows the analysis of the resultant mixture for formation of a bifunctional compound comprising each of the two molecules linked together by the linker.

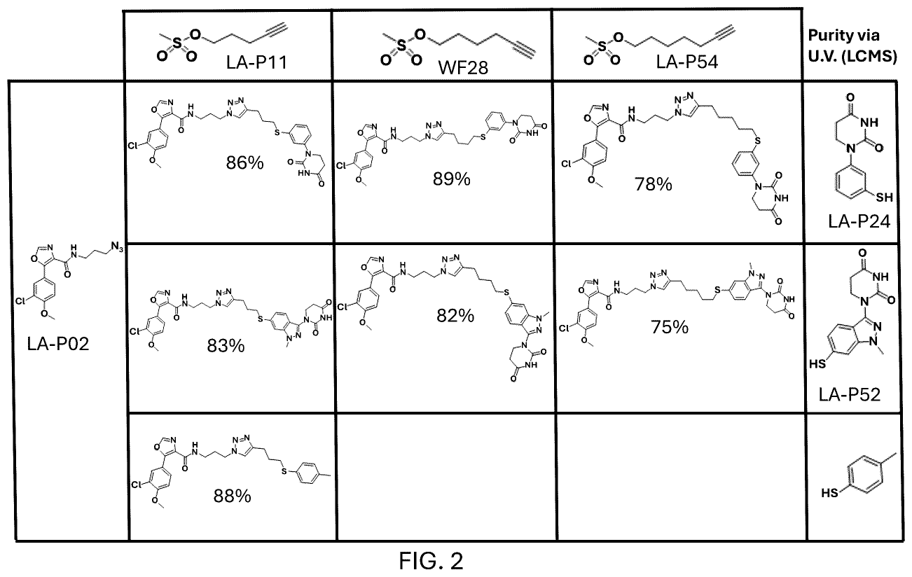

FIG. 2 shows the results a similar D2B method to that depicted in FIG. 1, but using the general orthogonal platform depicted in Scheme 2, and thus general experimental Method C described above. FIG. 2 further demonstrates the wide utility, effectiveness, and great structural diversity of the methods disclosed herein.

Therefore, the present investigators have shown that the methods of the present disclosure are operatively simple, result in high structural diversity of the bifunctional compounds, and are effective at screening reagents to assess their suitability in forming bifunctional compounds.

Furthermore, the present investigators have shown that bifunctional compounds manufactured using the method of the present disclosure may be effective in the uses of bifunctional compounds, for example as PROTACs in targeted protein degradation (TPD). That is to say, the methods of the present invention may additionally comprise contacting the resultant mixture with a target protein and/or a cell, for example GSK3p, and hence demonstrate that the method is a “direct-to-biology” method. GSK3p was targeted for degradation using a binder for GSK3p, and a binder for CRBN; the binders were comprised in molecules comprising moieties for reacting with the orthogonally reactive moieties of the linker. Table 1 (below) shows the results of a targeted degradation assay (DC₅₀, Dₘₐₓ) for the bifunctional compounds produced from the method depicted in Scheme 1, alongside the tabulated results from the method depicted in FIG. 1 (identified mass). The “identifier” identifies the well of the multi-well plate that has been sampled, e.g., AHD2B-1-A1 relates to the top left well in FIG. 1. The DC₅₀ provides an indication of the potency of the bifunctional compound in targeted degradation; Dₘₐₓ provides an indication of the maximum possible degradation. The use of these metrics is further described in Flexible Fitting of PROTAC Concentration-Response Curves with Changepoint Gaussian Processes, Semenova et al., SLAS Discovery, 2021, 26, 1212-1224.

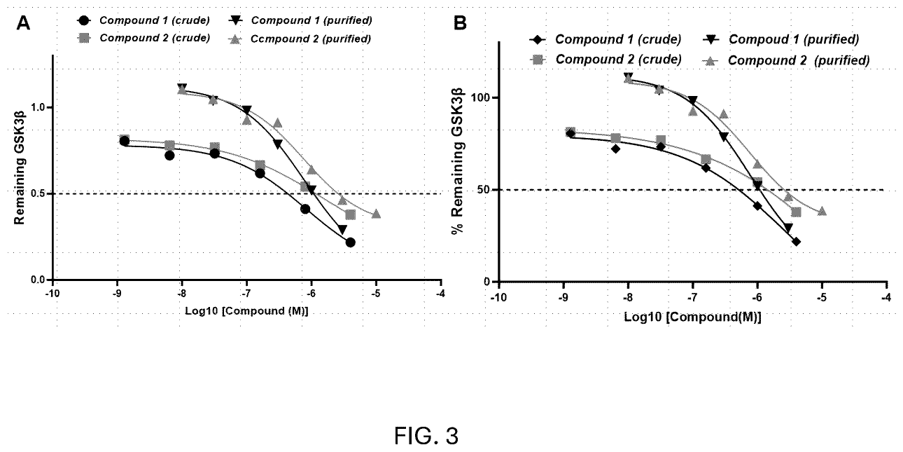

Additionally, FIG. 3 shows that the ‘crude’ compounds, that is to say, bifunctional compounds manufactured according to the method of the disclosure and hence not necessarily subjected to any chromatographic purification per se, performed similarly well in TPD to purified bifunctional compounds. Note that “Compound 1” and “Compound 2” of FIG. 3A and FIG. 3B refer to the bifunctional compounds of well ‘D7’ and well ‘D1’ of FIG. 1, respectively. This demonstrates that the purity of the bifunctional compounds produced using the methods disclosed herein is sufficient to demonstrate efficacy in the uses of bifunctional compounds, notwithstanding any high-throughput purification such as an ion-exchange system.

TABLE 1
Results of one-pot plate-based array method
and subsequent biological testing.

| | MW | Identified | DC₅₀ | Dₘₐₓ |
|---|---|---|---|---|
| Identifier | (g/mol⁻¹) | mass | (μM) | (%) |
| AHD2B-1-A1 | 704.2 | 704 | >10 | <30 |
| AHD2B-1-A2 | 739.2 | 738.9 | >10 | <30 |
| AHD2B-1-A3 | 768.3 | 767.9 | >10 | <30 |
| AHD2B-1-A4 | 796.3 | 796 | >10 | <30 |
| AHD2B-1-A5 | 677.1 | 677 | >10 | <30 |
| AHD2B-1-A6 | 794.3 | 794.308 | >10 | <30 |
| AHD2B-1-A7 | 718.2 | 718 | >10 | <30 |
| AHD2B-1-A8 | 739.2 | 738.9 | >10 | <30 |
| AHD2B-1-A9 | 732.3 | 732 | >10 | <30 |
| AHD2B-1-A10 | 750.2 | 749.9 | >10 | <30 |
| AHD2B-1-A11 | 780.3 | 780.281 | >10 | <30 |
| AHD2B-1-B1 | 972.6 | 972 | >10 | <30 |
| AHD2B-1-B2 | 1008 | 1007.1 | >10 | <30 |
| AHD2B-1-B3 | 1037 | 1036.2 | >10 | <30 |
| AHD2B-1-B4 | 1065 | 1064.2 | >10 | <30 |
| AHD2B-1-B5 | 945.5 | 945 | >10 | <30 |
| AHD2B-1-B6 | 1063 | 1062.2 | >10 | <30 |
| AHD2B-1-B8 | 1008 | 1007 | >10 | <30 |
| AHD2B-1-B9 | 1001 | 1000.1 | >10 | <30 |
| AHD2B-1-B10 | 1019 | 1018 | >10 | <30 |
| AHD2B-1-B11 | 1049 | 1048.1 | >10 | <30 |
| AHD2B-1-B12 | 958.6 | 958 | >10 | <30 |
| AHD2B-1-C1 | 764.2 | 763.9 | >10 | <30 |
| AHD2B-1-C2 | 799.2 | 799 | >10 | <30 |
| AHD2B-1-C3 | 828.3 | 828 | 1.8 | 37 |
| AHD2B-1-C4 | 856.3 | 856 | <0.25 | 53 |
| AHD2B-1-C5 | 737.2 | 736.9 | >10 | <30 |
| AHD2B-1-C6 | 854.3 | 854 | 1.6 | 59 |
| AHD2B-1-C7 | 778.3 | 778 | >10 | <30 |
| AHD2B-1-C8 | 799.2 | 798.9 | >10 | <30 |
| AHD2B-1-C9 | 792.3 | 792 | 7.1 | 39 |
| AHD2B-1-C10 | 810.3 | 810 | >10 | <30 |
| AHD2B-1-C11 | 840.3 | 839.8 | 1.9 | 56 |
| AHD2B-1-C12 | 750.2 | 749.8 | >10 | <30 |
| AHD2B-1-D1 | 758.3 | 757.9 | 2.1 | 49 |
| AHD2B-1-D2 | 793.3 | 792.9 | <0.25 | 63 |
| AHD2B-1-D3 | 822.3 | 821.9 | <0.25 | 52 |
| AHD2B-1-D4 | 850.4 | 849.8 | <0.25 | 41 |
| AHD2B-1-D5 | 731.2 | 730.8 | 0.55 | 35 |
| AHD2B-1-D7 | 772.3 | 772 | 0.41 | 56 |
| AHD2B-1-D8 | 793.3 | 792.9 | <0.25 | 56 |
| AHD2B-1-D9 | 786.3 | 786 | 0.53 | 62 |
| AHD2B-1-D10 | 804.3 | 804 | >10 | <30 |
| AHD2B-1-D11 | 834.3 | 833.9 | 3.1 | 41 |
| AHD2B-1-E1 | 592.1 | 592 | >10 | <30 |
| AHD2B-1-E2 | 627.1 | 627 | >10 | <30 |
| AHD2B-1-E3 | 656.2 | 656 | >10 | <30 |
| AHD2B-1-E4 | 684.2 | 683.9 | >10 | <30 |
| AHD2B-1-E5 | 565.1 | 565 | >10 | <30 |
| AHD2B-1-E6 | 682.2 | 682 | >10 | <30 |
| AHD2B-1-E7 | 606.2 | 606 | >10 | <30 |
| AHD2B-1-E8 | 627.1 | 627 | 12 | 41 |
| AHD2B-1-E9 | 620.2 | 620 | >10 | <30 |
| AHD2B-1-E10 | 638.2 | 638 | >10 | <30 |
| AHD2B-1-E11 | 668.2 | 667.9 | >10 | <30 |
| AHD2B-1-E12 | 578.1 | 578 | >10 | <30 |
| AHD2B-1-F1 | 747.2 | 746.8 | >10 | <30 |
| AHD2B-1-F2 | 782.2 | 781.8 | >10 | <30 |
| AHD2B-1-F3 | 811.3 | 810.9 | >10 | <30 |
| AHD2B-1-F4 | 839.3 | 838.8 | 0.25 | 32 |
| AHD2B-1-F5 | 720.2 | 719.8 | >10 | <30 |
| AHD2B-1-F6 | 837.3 | 836.9 | 1.1 | 34 |
| AHD2B-1-F7 | 761.3 | 760.9 | >10 | <30 |
| AHD2B-1-F8 | 782.2 | 781.8 | >10 | <30 |
| AHD2B-1-F9 | 775.3 | 775 | >10 | <30 |
| AHD2B-1-F10 | 793.3 | 792.9 | >10 | <30 |
| AHD2B-1-F11 | 823.3 | 822.8 | >10 | <30 |
| AHD2B-1-F12 | 733.2 | 732.8 | >10 | <30 |
| AHD2B-1-G1 | 704.2 | 704 | >10 | <30 |
| AHD2B-1-G2 | 739.2 | 738.9 | >10 | <30 |
| AHD2B-1-G3 | 768.3 | 767.8 | >10 | <30 |
| AHD2B-1-G4 | 796.3 | 796 | >10 | <30 |
| AHD2B-1-G5 | 677.1 | 676.9 | >10 | <30 |
| AHD2B-1-G6 | 794.3 | 793.8 | >10 | <30 |
| AHD2B-1-G7 | 718.2 | 717.9 | >10 | <30 |
| AHD2B-1-G8 | 739.2 | 738.8 | >10 | <30 |
| AHD2B-1-G9 | 732.3 | 731.9 | >10 | <30 |
| AHD2B-1-G10 | 750.2 | 749.8 | >10 | <30 |
| AHD2B-1-G12 | 690.2 | 689.9 | >10 | <30 |

MW = molecular weight; DC₅₀ = half-maximal degradation concentration; Dₘₐₓ = maximal effect.

1. A one-pot method comprising:
(i) contacting:
(a) a linker comprising two orthogonally reactive moieties; and
(b) two molecules, one of which comprises a moiety for reacting with one of the two orthogonally reactive moieties, and the other of which comprises a moiety for reacting with the other of the two orthogonally reactive moieties;
and optionally:
(ii) analysing the resultant mixture for formation of a bifunctional compound comprising each of the two molecules linked together by the linker.

2. The method of claim 1, wherein the linker is of formula (I): 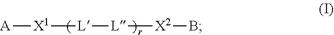
and
one of the two molecules comprises a moiety suitable for reacting with A, and the other comprises a moiety suitable for reacting with B;
wherein:
A is 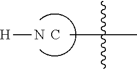
and B is ethynyl;
A is a nucleofuge and B is ethynyl; or
A is 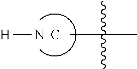
and B is a nucleofuge;
and wherein:
ring C is an aliphatic N-heterocycle optionally substituted with one or more selected from halo, C₁₋₆alkyl, C₁₋₆haloalkyl, C₁₋₆alkoxy, hydroxy, aryl, heteroaryl, and C₁₋₆haloalkoxy;
each X¹ and X² is optionally present and is any one selected from the group consisting of O(CH₂)ₛ and N(C₁₋₆alkyl)(CH₂)ₛ;
each L′ is independently selected from the group consisting of O(CH₂)ₜ, CH₂, heterocyclylene, arylene, and cycloalkylene, each optionally substituted with one or more substituents selected from halo, C₁₋₆alkyl, C₁₋₆haloalkyl, C₁₋₆alkoxy, hydroxy, aryl, heteroaryl, and C₁₋₆haloalkoxy;
L″ is optionally present and is selected from —O— and —N(C₁₋₆alkyl)-;
r is an integer from 1 to 20, s is an integer from 0 to 4, and t is an integer from 1 to 4.

3. The method of claim 2, wherein the linker is of formula (IIa) or (IIb): 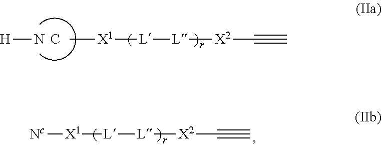
wherein:
N^{c} is the nucleofuge.

4. The method of claim 2, wherein ring C is:
(i) a 5- to 8-membered monocyclic ring or a bicyclic spiro moiety comprising 4- to 6-membered rings, each optionally substituted with one or more selected from halo, C₁₋₆alkyl, C₁₋₆haloalkyl, C₁₋₆alkoxy, hydroxy, aryl, heteroaryl, and C₁₋₆haloalkoxy; and/or
(ii) comprises no more than two nitrogen atoms;
optionally wherein ring C is selected from formulae (IIIa) to (IIIe): 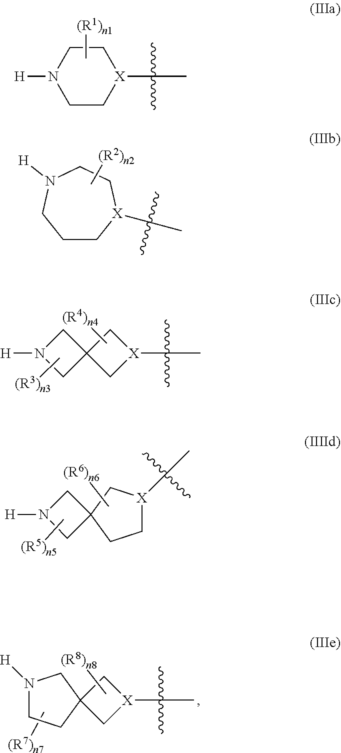
wherein:
X is N or CH;
each R¹ to R⁸ is independently selected from halo, C₁₋₆alkyl, C₁₋₆haloalkyl, C₁₋₆alkoxy, hydroxy, aryl, heteroaryl, and C₁₋₆haloalkoxy;
n1 is 0 to 4;
n2 is 0 to 5;
each n3, n4, n5 and n8 is independently selected from 0 to 2; and
each n6 and n7 is independently selected from 0 to 3.

5. The method of claim 2, wherein:
(i) the nucleofuge is selected from sulfonium, sulfonate and halo; and/or
(ii) X¹, L″ and X² are absent; and/or
(iii) each L′ is independently selected from the group consisting of O(CH₂)ₜ, CH₂, phenylene and pyridinene, each optionally substituted with one or more substituents selected from halo, C₁₋₆alkyl, C₁₋₆haloalkyl, C₁₋₆alkoxy, hydroxy, aryl, heteroaryl, and C₁₋₆haloalkoxy.

6. The method of claim 2, wherein: 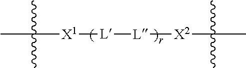
is selected from formulae (IVa) to (IVf): 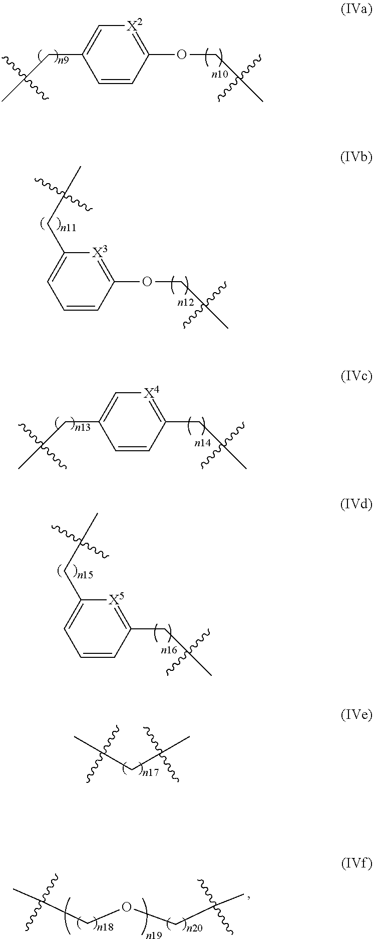
wherein:
each X² to X⁵ is independently selected from N and CH;
each n9 to n16, n18 and n20 is independently selected from 0 to 10; and
each n17 and n19 is independently selected from 1 to 10.

7. The method of claim 2, wherein the linker is selected from (Va) to (Vp):

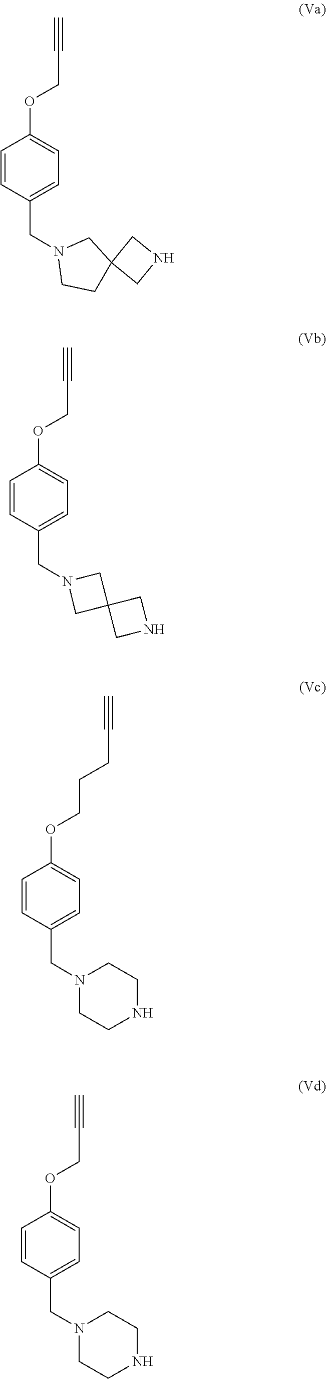 [SMILES: C#CCOc1ccc(CN2CCC3(CNC3)C2)cc1 ; C#CCOc1ccc(CN2CC3(CNC3)C2)cc1 ; C#CCCCOc1ccc(CN2CCNCC2)cc1 ; C#CCOc1ccc(CN2CCNCC2)cc1]

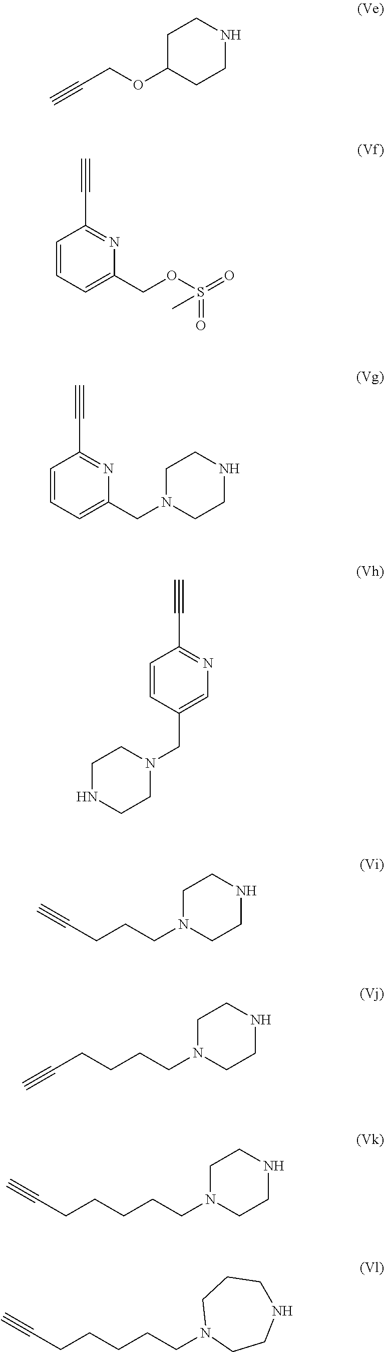

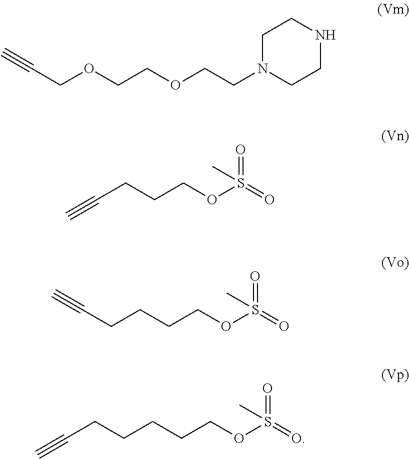

8. The method of claim 2, wherein:
(i) when A or B is 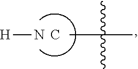
one of the two molecules comprises:
(a) a nucleofuge such as a sulfonium, sulfonate or halo moiety;
(b) a heteroaryl nucleofuge or aryl nucleofuge capable of undergoing:
a C—N cross-coupling reaction with the linker; and/or
a nucleophilic aromatic substitution reaction with the linker;
in order to displace the nucleofuge; or
(c) a carboxylic acid, an acyl chloride, a sulfonyl chloride, or an activated carboxylic acid, such as an N-hydroxysuccinimide ester;
(ii) when A or B is ethynyl, one of the two molecules comprises an azide or a nucleofuge such as a sulfonate or halo moiety; and
(ii) when A or B is a nucleofuge, such as a sulfonate or a halo moiety, one of the two molecules comprises a nucleophile capable of undergoing nucleophilic substitution with the linker, in order to displace the nucleofuge.

9. The method of claim 8, wherein the nucleophile is selected from thiol, hydroxy and amino.

10. The method of claim 1, wherein one or both of the molecules comprises/comprise binders for biological molecules.

11. The method of claim 1, wherein one of the molecules comprises an E3 ubiquitin ligase binder and the other comprises a target protein binder, such that the bifunctional compound is a proteolysis targeting chimera, optionally wherein:
(i) the E3 ubiquitin ligase binder is selected from a cereblon E3 ubiquitin ligase (CRBN) binder, an IAP E3 ubiquitin ligase binder, a Von Hippel-Lindau E3 ubiquitin ligase (VHL) binder, DDB1, CUL4 Associated Factor 1 (DCAF1), kelch domain-containing protein 2 (KLHDC2), and a mouse double minute 2 homologue (MDM2) ubiquitin ligase binder; and/or
(ii) the target protein binder is selected from a kinase inhibitor, a phosphatase inhibitor, a binder of a BET bromodomain-containing protein, an HDM2/MDM2 inhibitor, a heat shock protein 90 inhibitor, an HDAC inhibitor, and a human lysine methyltransferase inhibitor.

12. The method of claim 11, wherein, where the analysing shows formation of the proteolysis targeting chimera, the method further comprises contacting the resultant mixture with a target protein and/or a cell.

13. A linker of formula (1): 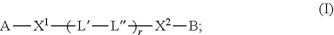
wherein:
A is 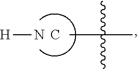
wherein ring C is a bicyclic spiro moiety comprising 4- to 6-membered aliphatic N-heterocyclic rings and optionally substituted with one or more selected from halo, C₁₋₆alkyl, C₁₋₆haloalkyl, C₁₋₆alkoxy, hydroxy, aryl, heteroaryl, and C₁₋₆haloalkoxy, and B is ethynyl or a nucleofuge;
each X¹ and X² is optionally present and is any one selected from the group consisting of O(CH₂)ₛ and N(C₁₋₆alkyl)(CH₂)ₛ;
each L′ is independently selected from the group consisting of O(CH₂)ₜ, CH₂, heterocyclylene, arylene, and cycloalkylene, each optionally substituted with one or more substituents selected from halo, C₁₋₆alkyl, C₁₋₆haloalkyl, C₁₋₆alkoxy, hydroxy, aryl, heteroaryl, and C₁₋₆haloalkoxy;
L″ is optionally present and is selected from —O— and —N(C₁₋₆alkyl)-; and
r is an integer from 1 to 20, s is an integer from 0 to 4, and t is an integer from 1 to 4.

14. The linker of claim 13, wherein A is selected from formulae (2a) to (2c): 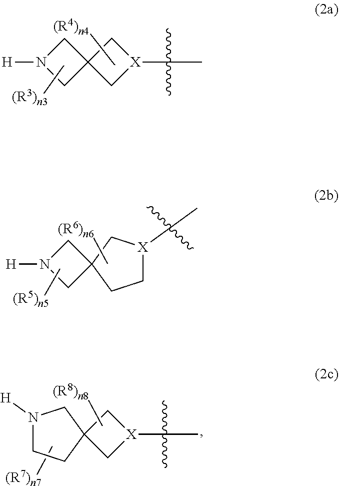
wherein:
X is N or CH;
each R³ to R⁸ is independently selected from halo, C₁₋₆alkyl, C₁₋₆haloalkyl, C₁₋₆alkoxy, hydroxy, aryl, heteroaryl, and C₁₋₆haloalkoxy;
each n3, n4, n5 and n8 is independently selected from 0 to 2; and
each n6 and n7 is independently selected from 0 to 3.

15. The linker of claim 13, wherein:
(i) the nucleofuge is selected from sulfonate and halo; and/or
(ii) X¹, L″ and X² are absent; and/or
(iii) each L′ is independently selected from the group consisting of O(CH₂)ₜ, CH₂, phenylene and pyridinene, each optionally substituted with one or more substituents selected from halo, C₁₋₆alkyl, C₁₋₆haloalkyl, C₁₋₆alkoxy, hydroxy, aryl, heteroaryl, and C₁₋₆haloalkoxy.

16. The linker of claim 13, wherein: 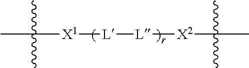
is selected from formulae (3a) to (3f): 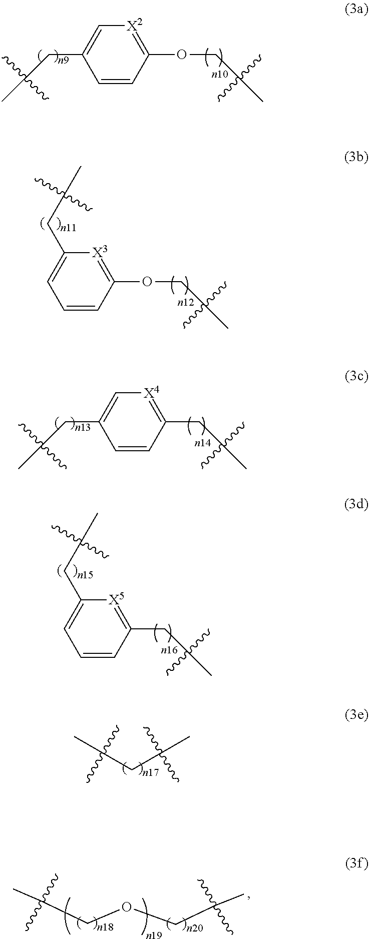
wherein:
each X² to X⁵ is independently selected from N and CH;
each n9 to n16, n18 and n20 is independently selected from 0 to 10; and
each n17 and n19 is independently selected from 1 to 10.

17. The linker of claim 13, wherein the linker is of formula (4a) or (4b):

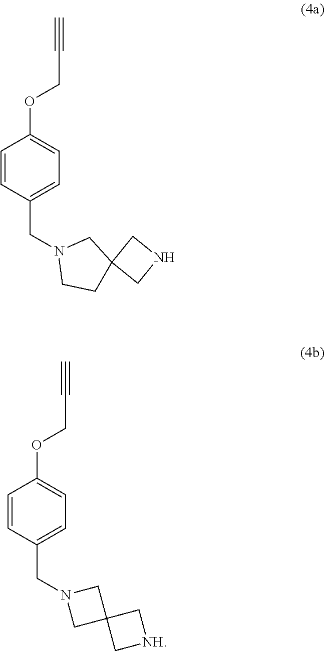 [SMILES: C#CCOc1ccc(CN2CCC3(CNC3)C2)cc1 ; C#CCOc1ccc(CN2CC3(CNC3)C2)cc1]

18. A method of manufacturing a bifunctional compound, optionally as a one-pot method, the method comprising:
(i) contacting:
(a) a linker of claim 13; and
(b) two molecules, one of which comprises a moiety for reacting with one of A and B, and the other of which comprises a moiety for reacting with the other of A and B;
and optionally:
(ii) analysing the resultant mixture for formation of a bifunctional compound comprising each of the two molecules linked together by the linker.

19. A method of targeted protein degradation or stabilisation, the method comprising:
(i) contacting:
(a) a linker of claim 13; and
(b) two molecules, one of which comprises a moiety for reacting with one of A and B, and the other of which comprises a moiety for reacting with the other of A and B, and wherein one of the molecules comprises a target protein binder for binding a target protein and the other comprises an E3 ubiquitin ligase or a deubiquitinase binder;
(ii) analysing the resultant mixture for formation of a bifunctional compound comprising each of the two molecules linked together by the linker; and
(iii) contacting the bifunctional compound with the target protein.

20. A kit comprising:
(i) a linker as defined in claim 13; and
(ii) a molecule comprising a moiety for reacting with A and/or a molecule comprising a moiety for reacting with B.
